SEQUENCE LISTING (54 sequences)

SEQ ID NO: 1 - protein, 15 aa, Artificial Sequence
  Neoantigen
NNPPGAYIFTVSAMD

SEQ ID NO: 2 - protein, 15 aa, Artificial Sequence
  Neoantigen
LYLAFPVAFCIFMVR

SEQ ID NO: 3 - protein, 15 aa, Artificial Sequence
  Neoantigen
PIPQEYVLFLCPTGP

SEQ ID NO: 4 - protein, 15 aa, Artificial Sequence
  Neoantigen
SHHHTMMVQAITFIF

SEQ ID NO: 5 - protein, 15 aa, Artificial Sequence
  Neoantigen
GIVFSYIHIISSILR

SEQ ID NO: 6 - protein, 15 aa, Artificial Sequence
  Neoantigen
SSQLHHSMYFFLSHL

SEQ ID NO: 7 - protein, 15 aa, Artificial Sequence
  Neoantigen
SGGPGSEKSLLLSKW

SEQ ID NO: 8 - protein, 15 aa, Artificial Sequence
  Neoantigen
VPPLRPNPALPPLPT

SEQ ID NO: 9 - protein, 15 aa, Artificial Sequence
  Neoantigen
PLRCSSPLAAPTFLR

SEQ ID NO: 10 - protein, 15 aa, Artificial Sequence
  Neoantigen
KAISNPHMYAVDMSS

SEQ ID NO: 11 - protein, 15 aa, Artificial Sequence
  Neoantigen
ENIHGPWDEAEIKKD

SEQ ID NO: 12 - protein, 15 aa, Artificial Sequence
  Neoantigen
FSPTRDVILYYVNLK

SEQ ID NO: 13 - protein, 15 aa, Artificial Sequence
  Neoantigen
FFGPLILFYTWPFPT

SEQ ID NO: 14 - protein, 15 aa, Artificial Sequence
  Neoantigen
MGPPGGFQEFNFIVP

SEQ ID NO: 15 - protein, 15 aa, Artificial Sequence
  Neoantigen
DTISRAVFPLAFLIF

SEQ ID NO: 16 - protein, 15 aa, Artificial Sequence
  Neoantigen
SFKYYAHATSLAGHL

SEQ ID NO: 17 - protein, 15 aa, Artificial Sequence
  Neoantigen
LNMPELNITVYKDFS

SEQ ID NO: 18 - protein, 15 aa, Artificial Sequence
  Neoantigen
GIRKAKKRNYAFLWD

SEQ ID NO: 19 - protein, 15 aa, Artificial Sequence
  Neoantigen
PHLKDWQIFHQALLR

SEQ ID NO: 20 - protein, 15 aa, Artificial Sequence
  Neoantigen
GLGKHGFIMEMNAFG

SEQ ID NO: 21 - protein, 15 aa, Artificial Sequence
  Neoantigen
NHAFKCKHCGKPFGT

SEQ ID NO: 22 - protein, 15 aa, Artificial Sequence
  Neoantigen
LDKHWHLECFKCKTC

SEQ ID NO: 23 - protein, 15 aa, Artificial Sequence
  Neoantigen
PCVFLVNCLPFCRTH

SEQ ID NO: 24 - protein, 15 aa, Artificial Sequence
  Neoantigen
KVDIMPPHVFFYVDK

SEQ ID NO: 25 - protein, 15 aa, Artificial Sequence
  Neoantigen
KAEILSSLGALYYNT

SEQ ID NO: 26 - protein, 15 aa, Artificial Sequence
  Neoantigen
PHGYEIKFIFGIPLD

SEQ ID NO: 27 - protein, 15 aa, Artificial Sequence
  Neoantigen
IRENFLFLDVFFEAL

SEQ ID NO: 28 - protein, 15 aa, Artificial Sequence
  Neoantigen
QHDKEQKMIGSVSQS

SEQ ID NO: 29 - protein, 15 aa, Artificial Sequence
  Neoantigen
QENTCNITIEGLDAE

SEQ ID NO: 30 - protein, 15 aa, Artificial Sequence
  Neoantigen
VEDPVYQVISETSRD

SEQ ID NO: 31 - protein, 15 aa, Artificial Sequence
  Neoantigen
LGFFKQNEHQNLLFV

SEQ ID NO: 32 - protein, 15 aa, Artificial Sequence
  Neoantigen
TKFILVEFPGSLSMR

SEQ ID NO: 33 - protein, 15 aa, Artificial Sequence
  Neoantigen
ILLENTDLSALWYLY

SEQ ID NO: 34 - protein, 15 aa, Artificial Sequence
  Neoantigen
SEKLSHLKITGDSFL

SEQ ID NO: 35 - protein, 15 aa, Artificial Sequence
  Neoantigen
KQEFVKPKIVSREGG

SEQ ID NO: 36 - protein, 15 aa, Artificial Sequence
  Neoantigen
DCFSTNKLLTYEALG

SEQ ID NO: 37 - protein, 15 aa, Artificial Sequence
  Neoantigen
KAIEKEMKVTALPPK

SEQ ID NO: 38 - protein, 15 aa, Artificial Sequence
  Neoantigen
DKEFVKFNRMHAVNG

SEQ ID NO: 39 - protein, 15 aa, Artificial Sequence
  Neoantigen
ARALILLPTRELALQ

SEQ ID NO: 40 - protein, 15 aa, Artificial Sequence
  Neoantigen
WQPQSHSRQGHGTSP

SEQ ID NO: 41 - protein, 15 aa, Artificial Sequence
  Neoantigen
SIHLLTSRATNSQLF

SEQ ID NO: 42 - protein, 15 aa, Artificial Sequence
  Neoantigen
QVIALHCSQAQSSVS

SEQ ID NO: 43 - protein, 15 aa, Artificial Sequence
  Neoantigen
LRCMGNNGICRASCK

SEQ ID NO: 44 - protein, 15 aa, Artificial Sequence
  Neoantigen
WITPLDHLVAFPTRV

SEQ ID NO: 45 - protein, 15 aa, Artificial Sequence
  Neoantigen
AIKTLKSGHMDRQRR

SEQ ID NO: 46 - protein, 15 aa, Artificial Sequence
  Neoantigen
ALFDGDSHLSTENPA

SEQ ID NO: 47 - protein, 15 aa, Artificial Sequence
  Neoantigen
PSPTVLRAHAEMALD

SEQ ID NO: 48 - protein, 15 aa, Artificial Sequence
  Neoantigen
DMLSQTNCFVSLWLP

SEQ ID NO: 49 - protein, 15 aa, Artificial Sequence
  Spaceholder
PVSKMRMATPLLMQA

SEQ ID NO: 50 - protein, 13 aa, Artificial Sequence
  Spaceholder
AAMAAAAAAAMAA

SEQ ID NO: 51 - protein, 13 aa, Artificial Sequence
  Spaceholder
AAMAAAAAAAAAA

SEQ ID NO: 52 - protein, 13 aa, Artificial Sequence
  Spaceholder
AAFAAAAAAAAAA

SEQ ID NO: 53 - protein, 13 aa, Artificial Sequence
  Spaceholder
ASMSAASAASMAA

SEQ ID NO: 54 - protein, 13 aa, Artificial Sequence
  Spaceholder
  misc_feature (3)..(3): Xaa can be any naturally occurring amino acid
  misc_feature (11)..(11): Xaa can be any naturally occurring amino acid
AAXAAAAAAAXAA

(19) United States
(12) Patent Application Publication
(10) Pub. No.: US 2023/0193203 A1
(43) Pub. Date: Jun. 22, 2023

(54) NEOANTIGEN-INFORMED TUMOR-INFILTRATING LYMPHOCYTE CANCER IMMUNOTHERAPY

(71) Applicant: THE TRANSLATIONAL GENOMICS RESEARCH INSTITUTE, Phoenix, AZ (US)

(72) Inventors: Sunil Sharma, Phoenix, AZ (US); John Altin, Phoenix, AZ (US); Raffaella Soldi, Phoenix, AZ (US); Jeffrey Trent, Phoenix, AZ (US); Timothy McDaniel, Phoenix, AZ (US)

(21) Appl. No.: 17/922,315

(22) PCT Filed: Apr. 30, 2021
(86) PCT No.: PCT/US21/30331

§ 371 Date: Oct. 28, 2022

Related U.S. Application Data
(60) Provisional application No. 63/018,059, filed on Apr. 30, 2020.

(51) Int. Cl. C12N 5/0783; C12Q 1/6886; C12Q 1/6881; C12N 15/10; A61P 35/00; A61P 1/00; A61K 35/17; A61K 38/20; A61K 45/06; A61P 17/00; A61P 11/00; A61P 1/18

(52) U.S. Cl. CPC C12N 5/0638; C12Q 1/6886; C12Q 1/6881; C12N 15/1068; A61P 35/00; A61P 1/00; A61K 35/17; A61K 38/2013; A61K 45/06; A61P 17/00; A61P 11/00; A61P 1/18; C12N 2501/998; C12Q 2600/156; C12Q 2600/158; C12N 2501/2302

(57) ABSTRACT

The present invention provides a method for educating and expanding tumor infiltrating lymphocytes (TILs) into a therapeutic population of TILs for adoptive cell immunotherapy. Also provided are methods of treating cancer patients with the therapeutic population of TILs.

### CROSS REFERENCE TO RELATED APPLICATIONS

This application claims the benefit of U.S. Provisional Patent Application No. 63/018,059, filed on Apr. 30, 2020, the contents of which are incorporated herein by reference in its entirety.

### REFERENCE TO SEQUENCE LISTING SUBMITTED ELECTRONICALLY

The official copy of the sequence listing is submitted electronically via EFS-Web as an ASCII-formatted sequence listing with a file named “91482-242PCT_Sequence_Listing.txt” created on Apr. 30, 2021, and having a size of 13,002 bytes, is filed concurrently with the specification. The sequence listing contained in this ASCII-formatted document is part of the specification and is herein incorporated by reference in its entirety.

### TECHNICAL FIELD

This application relates to methods for educating and expanding tumor infiltrating lymphocytes (TILs) into a therapeutic population of TILs for adoptive cell immunotherapy and related methods of treating cancer patients.

### BACKGROUND

Immunotherapy is a burgeoning approach to cancer therapy in which T cell-mediated responses against tumor antigens act to eliminate tumor cells. Neoantigens are Major Histocompatibility Complex (MHC)-presented peptides of novel sequence that are formed by somatic mutation and represent an especially promising immunotherapeutic target, since they appear exclusively on the tumor and so cause minimal off-target effects. TIL therapy refers to a type of autologous cellular immunotherapy in which TILs are isolated from surgically-resected tumor specimens, expanded in vitro, and then reinfused into the patient, typically following a lymphodepleting conditioning regimen. While TIL therapy has achieved some impressive responses against a range of cancers^{1,2,3,4}, its success remains sporadic⁵.

On the other hand, there are several reasons in principle to think that TIL therapy can be a broadly applicable therapeutic. First, it is known that the majority of tumors are infiltrated by neoantigen-specific T cells, albeit sometimes at very low frequencies⁶. Second, it has been shown that the expansion and infusion of a highly pure TIL product comprising CD4 or CD8 T cells reactive to a single neoantigen is sufficient to mediate tumor regression^{7,8}. Thus, there is a need for improved methods of generating TIL therapies that effectively treat cancer and, particularly, metastatic cancer.

### SUMMARY

The present invention provides a method for expanding tumor infiltrating lymphocytes (TILs) into a therapeutic population of TILs for adoptive cell immunotherapy comprising: (a) separating a first population of TILs from tumor cells obtained from a tumor resected from a patient; (b) detecting a plurality of patient-specific tumor mutations in the tumor cells with a genomic analysis of tumor DNA and/or RNA and normal DNA and/or RNA from the patient; (c) identifying neoantigens resulting from the somatic mutations that demonstrate specific binding with human leukocyte antigen (HLA) proteins or fragments thereof from the first population of TILs; (d) incubating the neoantigens with the first population of TILs to generate a second population of TILs enriched with lymphocytes recognizing the neoantigens; and (e) expanding the second population of TILs into a therapeutic population of TILs for adoptive cell immunotherapy.

In certain aspects, detecting a plurality of patient-specific tumor mutations comprises genomic profiling with next generation sequencing of a targeted gene panel. In one aspect, the genomic profiling comprises whole genome profiling, whole exome profiling, and/or transcriptome profiling.

In other aspects, the genomic analysis comprises identifying a plurality of patient-specific tumor mutations in expressed genes by nucleic acid sequencing of tumor and normal samples from the patient and the mutations are present in the genome of cancer cells of the patient but not in normal cells from the subject.

In yet other aspects, the plurality of patient-specific tumor mutations comprises a point mutation, splice-site mutation, frameshift mutation, read-through mutation, gene-fusion mutation, insertion, deletion, or a combination thereof; and the plurality of patient-specific tumor mutations encodes at least one mutant polypeptide having a tumor-specific neoepitope which binds to an HLA protein or fragment thereof with a greater affinity than a wild-type polypeptide.

In some aspects, the method further comprises identifying the MHC class 1 and 2 genotypes of the patient. In one aspect, identifying the MHC class 1 and 2 genotypes of the patient comprises analysis of whole exome sequencing (WES) and/or RNA sequencing from tumor and/or normal tissue.

In certain aspects, identifying the neoantigens comprises: (i) providing a library of peptide constructs, wherein each peptide construct of the library comprises a peptide portion and an identifying nucleic acid portion that identifies the peptide portion, and the peptide portion of at least one of the peptide constructs is capable of specific binding to the HLA proteins or fragments thereof; (ii) contacting the HLA proteins or fragments thereof with the library of peptide constructs; (iii) separating the at least one peptide construct comprising a peptide portion capable of specific binding to the HLA proteins or fragments thereof from peptide constructs comprising a peptide portion not capable of specific binding to the HLA proteins or fragments thereof; (iv) sequencing all or a portion of the identifying nucleic acid portion of the at least one peptide construct capable of specific binding to the HLA proteins or fragments thereof.

In some aspects, the library of peptide constructs comprises variant peptides designed from an analysis of the plurality of patient-specific tumor mutations predicting the impact of each mutation on a corresponding protein and excluding silent mutations and mutations in noncoding regions. In one aspect, the variant peptides comprise mutations predicted to impact the structure of the corresponding protein.

In other aspects, identifying the neoantigens comprises (i) generating a genetically encoded combinatorial library of polypeptides with phage display, ribosomal display, mRNA display, biscistronic DNA display, P2A DNA display, CIS display, yeast display, or bacterial display, wherein the combinatorial library comprises polypeptides linked to corresponding nucleic acid molecules encoding the polypeptides; (ii) contacting the combinatorial library with the HLA proteins or fragments thereof; (iii) separating HLA proteins or fragments thereof demonstrating specific binding with the combinatorial library; and (iv) sequencing all or a portion of the nucleic acid molecules of the combinatorial library bound to the HLA proteins or fragments thereof to identify the neoantigens.

In one aspect, the combinatorial library of polypeptides comprises variant peptides designed from an analysis of the plurality of patient-specific tumor mutations predicting the impact of each mutation on a corresponding protein and excluding silent mutations and mutations in noncoding regions.

In certain aspects, specific binding between neoantigens and HLA proteins or fragments thereof from the first population of TILs is determined by: (i) culturing a cell transformed with at least one nucleic molecule comprising a nucleotide sequence encoding: an MHC class II component comprising at least a portion of an MHC class II α chain and at least a portion of an MHC class II β chain, such that the MHC class II α chain and MHC class II β chain form a peptide binding groove; and a spaceholder molecule and a first processable linker, wherein the spaceholder molecule is linked to the MHC class II component by the processable linker and the spaceholder molecule binds within the peptide binding groove thereby hindering the binding of any other peptide within the peptide binding groove; the step of culturing being conducted to produce the MHC class II component; (ii) recovering the MHC class II component; (iii) processing the processable linker, thereby releasing the spaceholder molecule from the peptide binding groove; (iv) incubating the MHC class II component in the presence of a neoantigen, wherein the incubation facilitates the binding of the neoantigen to the peptide binding groove; (v) recovering the MHC class II component that has bound the neoantigen.

In one aspect, the spaceholder molecule has the consensus sequence AAXAAAAAAAXAA (SEQ ID NO: 78). In another aspect, the spaceholder molecule is selected from the group consisting of PVSKMRMATPLLMQA (SEQ ID NO: 73); AAMAAAAAAAMAA (SEQ ID NO: 74); AAMAAAAAAAAAA (SEQ ID NO: 75); AAFAAAAAAAAAA (SEQ ID NO: 76); and ASMSAASAASMAA (SEQ ID NO: 77).

In some aspects, the processable linker is linked to the MHC class II α chain of the MHC class II component. In other aspects, recovering the MHC class II component with bound neoantigen comprises affinity chromatography with an antibody recognizing the MHC class II component.

In certain aspects, specific binding between neoantigens and HLA proteins or fragments thereof from the first population of TILs is determined by phage display, the HLA proteins or fragments thereof are expressed on the surface of a phage, and the neoantigens are incubated with the phage to assay specific binding.

In one aspect, the method further comprises an in silico analysis to determine specific binding between neoantigens and MHC class I proteins or fragments thereof, wherein the in silico analysis comprises applying a computational algorithm to predict relative binding to MHC I proteins based on the peptide sequences of the neoantigens.

In another aspect, the method further comprises removing TILs expressing a marker selected from the group consisting of PD1, TIM-3, LAG-3, CTLA-4, and combinations thereof from the first population of TILs and/or the second population of TILs via cell sorting to enrich non-exhausted TILs in the populations.

In yet another aspect, incubating the neoantigens with the first population of TILs further comprises contacting the first population of TILs with at least one cytokine. In one aspect, the at least one cytokine comprises interleukin-2 (IL-2).

In some aspects, expanding the second population of TILs into a therapeutic population of TILs comprises injection of the second population of TILs into a lymph node area of the patient, into the thymus of the patient, and/or systemically into the patient via intravenous administration.

In other aspects, expanding the second population of TILs into a therapeutic population of TILs comprises supplementing the cell culture medium of the second population of TILs with IL-2, optionally OKT-3, and feeder cells.

In yet other aspects, expanding the second population of TILs into a therapeutic population of TILs comprises: (i) performing a first expansion by culturing the second population of TILs in a cell culture medium comprising IL-2, and optionally OKT-3, to produce a third population of TILs, wherein the first expansion is performed in a closed container providing a first gas-permeable surface area, wherein the first expansion is performed for about 3-14 days to obtain the third population of TILs, wherein the third population of TILs is at least 50-fold greater in number than the second population of TILs, and wherein the transition occurs without opening the system; and, optionally, (ii) performing a second expansion by supplementing the cell culture medium of the third population of TILs with additional IL-2, optionally OKT-3, and feeder cells, to produce a fourth population of TILs, wherein the second expansion is performed for about 7-14 days to obtain the fourth population of TILs, wherein the fourth population of TILs is a therapeutic population of TILs, wherein the third expansion is performed in a closed container providing a second gas-permeable surface area, and wherein the transition occurs without opening the system. In one aspect, the feeder cells are antigen presenting cells (APCs) or irradiated peripheral blood mononuclear cells (PBMCs).

In another aspect, the method further comprises performing an immune infiltration assay with organoids derived from tumor cells from the patient to confirm enhanced infiltration of the organoids with the second population of TILs compared to the first population of TILs.

The present invention also provides a cryopreserved composition comprising a therapeutic population of TILs as disclosed herein, a cryoprotectant medium comprising DMSO, and an electrolyte solution.

In some aspects, the cryopreserved composition further comprises one or more stabilizers and one or more lymphocyte growth factors. In one aspect, the one or more stabilizers comprise Human Serum Albumin (HSA) and the one or more lymphocyte growth factors comprise IL-2.

The present invention also relates to a method of treating a subject with cancer, the method comprising administering an effective amount of a therapeutic population of TILs as disclosed herein to a patient in need thereof.

In some aspects, prior to administering an effective amount of the therapeutic population of TILs, a non-myeloablative lymphodepletion regimen is administered to the patient. In one aspect, the non-myeloablative lymphodepletion regimen comprises the steps of administration of cyclophosphamide at a dose of 60 mg/m²/day for two days followed by administration of fludarabine at a dose of 25 mg/m²/day for five days.

In other aspects, the method further comprises the step of treating the patient with a high-dose IL-2 regimen starting on the day after administration of the therapeutic population of TILs. In one aspect, the high-dose IL-2 regimen comprises 600,000 or 720,000 IU/kg administered as a 15-minute bolus intravenous infusion every eight hours until tolerance.

In certain aspects, the method further comprises contacting the therapeutic population of TILs with or administering to the patient an immune checkpoint inhibitor. In one aspect, the checkpoint inhibitor comprises a CD27, CD28, CD40, CD 122, CD137, OX40, GITR, ICOS, A2AR, B7-H3, B7-H4, BTLA, CTLA-4, IDO, KIR, LAG3, PD-1, PD-L1, TIM-2, 4-1BB, or VISTA inhibitor or a combination thereof. In another aspect, the checkpoint inhibitor comprises ipiliumab (anti-CTLA-4), penbrolizumab (anti-PD-L1), nivolumab (anti-PD-L1), atezolizumab (anti-PD-L1), duralumab (anti-PD-L1), or a combination thereof.

In some aspects, the cancer is selected from the group consisting of melanoma, ovarian cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), renal cancer, and renal cell carcinoma. In other aspects, the cancer is selected from the group consisting of melanoma, HNSCC, cervical cancers, NSCLC, and triple negative breast cancer (TNBC).

In yet other aspects, the therapeutic population of TILs are administered intravenously, intraperitoneally, subcutaneously, intramuscularly, or intratumorally.

### BRIEF DESCRIPTION OF THE DRAWINGS

FIG. 1 depicts a comparison of the traditional method to isolating therapeutic tumor-infiltrating lymphocytes (TILs) and the described improved method that uses genomic tools to enrich TILs.

FIG. 2 depicts a more detailed outline of the workflow of the method described herein beginning with collecting lymphocytes from a patient.

FIG. 3 depicts a simplified timeline for the workflow shown in FIG. 2 with approximate timing requirements shown.

FIG. 4 depicts a more detailed timeline for the workflow shown in FIG. 2 with approximate timing requirements shown.

FIG. 5 depicts PepSeq as a platform for efficient synthesis and analysis of large libraries of DNA-barcoded peptides. The peptide-DNA library depicted shows a puromycin (“Puro”) adaptor that facilitates linking of the DNA to the encoded peptide.

FIG. 6 depicts analysis of tumor exomes to identify candidate neoantigens, characterization of the candidate neoantigens with the MHC-PepSeq assay, and evaluation of T cell response to the neoantigens that bound to HLA proteins in the MHC-PepSeq assay.

FIG. 7 depicts a PepSeq library design for the analysis of mutations identified from an analysis of tumor exomes.

FIG. 8 depicts an embodiment of the MHC-PepSeq assay used to identify neoantigens that bind to MHC Class II molecules. In the control group (“uncleaved”), the CLIP peptide is not removed from the HLA construct and candidate neoantigens bind non-specifically while in the test group (“cleaved”) removal of the CLIP peptide opens the recognition site of the HLA construct allowing for specific binding of candidate neoantigens.

FIG. 9 depicts the identification of enriched peptides (i.e., neoantigens) from 2 human patients (i.e., TG0006 and TG00013). The numbers of mutations identified in each patient with exome analysis are shown under each patient ID. The human leukocyte antigen serotypes of the HLA complexes used in the binding assays are identified in the upper left-hand corner of each graph. Enriched peptides are shown as larger data points.

FIG. 10 depicts additional data sets identifying enriched peptides (i.e., neoantigens) from human patient TG00013. Enriched peptides appear as data points in the upper portion of each graph corresponding to those peptides with a log average % read with cleaved HLA complexes closer to 0 (i.e., closer to 100% binding with cleaved HLA complexes).

FIG. 11 depicts the amino acid sequences of the enriched peptides, the corresponding mutation for each peptide, and the human leukocyte antigen serotype of the HLA complex with which the peptide specifically binds for human patient TG00013.

FIG. 12 depicts a comparison of MHC-PepSeq versus in silico prediction to identify neoantigens. The IEDB consensus sequences used for the in silico analysis are obtained from the Immune Epitope Database and Analysis Resource (IEDB), a public resource maintained by the National Institute of Allergy and Infectious Diseases (NIAID).

FIG. 13 depicts the generation of a spheroid/organoid from a suspension of tumor cells.

FIGS. 14A, 14B, and 14C depict an infiltration assay workflow. In FIG. 14A, after organoids are formed from tumor cell suspensions in a round bottom plate, a permeable support is inserted, and enriched TILs are allowed to migrate through the support to infiltrate the organoid. FIG. 14B depicts the production of a three-dimensional image of the organoid with Z-stacking. FIG. 14C depicts quantification of the infiltration of the lymphocytes into the organoid.

FIG. 15 depicts TIL infiltration into an organoid derived from patient TG00013 who suffered from melanoma.

FIG. 16 depicts another dataset evaluating TIL infiltration into an organoid derived from patient TG00013 along with a statistical analysis of the differences between treatment groups.

FIG. 17 depicts the specific infiltration of TILs into tumor cells compared to general, uninvolved (“GU”) tissue from patient TG00013.

FIG. 18 depicts one embodiment of a workflow for a NeoTIL analysis and therapy with a CT26 colon carcinoma mouse. In another embodiment, the subject (e.g., mouse or human) experiences lymphodepletion prior to Step IV.

FIG. 19 depicts enriched peptides (i.e., neoantigens) from a CT26 colon carcinoma mouse. Enriched peptides appear as data points in the upper portion of the graph corresponding to those peptides with a log average % read with cleaved mouse MHC class I or class II alloantigens closer to 0 (i.e., closer to 100% binding with cleaved mouse alloantigens).

FIG. 20 depicts the amino acid sequences of the enriched peptides, the corresponding mutation for each peptide, and the mouse MHC class I or class II alloantigen with which the peptide specifically binds for the CT26 colon carcinoma mouse.

FIG. 21 depicts microscopic images of TILs co-cultured with DMSO, IL-2, peptides (i.e., neoantigens), or peptides with IL-2 at Day 1 and at Day 7.

FIG. 22 depicts microscopic images from an immune infiltration assay in CT26 mouse tumor organoids. The following treatments were administered to the organoid: 1) TILs; 2) TILs+IL-2; 3) TILs+peptides (i.e., neoantigens); 4) TILs+IL-2+peptides (i.e., neoantigens); and 5) TILs+DMSO.

FIG. 23 depicts quantification of the TIL infiltration into an organoid derived from a CT26 mouse tumor as shown in the images of FIG. 22. TILs were stained with Invitrogen CELLTRACKER™ CM-DiI, a red fluorescent dye well suited for monitoring multigenerational cell movement or location. The dye facilitates the quantification of TILs infiltration into the organoids. Imaging analysis was performed by Cytation 5 software. A statistical analysis of the differences between treatment groups is also shown, with significant differences marked with asterisks.

FIG. 24 depicts average tumor volume in CT26 colon carcinoma mice after intratumoral (“IT”) administration of 1) TILs; 2) TILs+IL-2; 3) TILs+peptides (neoantigens); 4) TILs+IL-2+peptides (neoantigens); and 5) vehicle.

FIG. 25 depicts average tumor volume in CT26 colon carcinoma mice after intravenous (“IV”) administration of 1) TILs+IL-2; 2) TILs+peptides (neoantigens); 3) TILs+IL-2+peptides (neoantigens); and 4) TILs+vehicle.

FIGS. 26A-26E depict the efficacy of Neo-TILs™ in melanoma. TIL infiltration was measured in a melanoma patient's tumor organoids (FIGS. 26A, 26B). FIG. 26B depicts immune infiltration assay in a melanoma patient's organoids from tumor (black) or general uninvolved (GU) tissue (grey). Levels of IFNγ (FIG. 26C), TNFα (FIG. 26D), and Granzyme B (FIG. 26E) were measured by MesoScale Discovery (MSD) assay in TILs conditioned medium.

FIGS. 27A-27K depict the efficacy of Neo-TILs™ in melanoma. TIL infiltration was measured in a melanoma patient's tumor organoids (FIG. 27A). qPCR assay measured relative expression of IFNγ (FIG. 27B), Granzyme B (FIG. 27C), and Perforin (FIG. 27D) in TILs and conditioned medium. MSD assay measured levels of IFNγ (FIG. 27E), TNFα (FIG. 27F), and Granzyme B (FIG. 27G) in TILs and conditioned medium. qPCR assay also measured relative expression for exhaustion marker PD-1 (FIG. 27H), TIM-3 (FIG. 27I), LAG3 (FIG. 27J), and TIGIT (FIG. 27K).

FIGS. 28A-28G depict the efficacy of Neo-TILs™ in lung cancer. TIL infiltration was measured in a lung cancer patient's tumor organoids (FIGS. 28A and 28B). FIG. 26B depicts immune infiltration assay in a lung cancer patient's organoids from tumor (black) or GU tissue (grey). Levels of IFNγ (FIG. 28C), Granzyme B (FIG. 28B) were measured by MSD assay in TILs conditioned medium. qPCR assay measured relative expression of IFNγ (FIG. 28E), Granzyme B (FIG. 28F), and Perforin (FIG. 28G) in TILs conditioned medium.

FIGS. 29A-29K depict the efficacy of Neo-TILs™ in colon cancer. TIL infiltration was measured in a colon cancer patient's tumor organoids (FIG. 29A). qPCR assay measured relative expression of IFNγ (FIG. 29B), Granzyme B (FIG. 29C), and Perforin (FIG. 29D) in TILs and conditioned medium. MSD assay measured levels of IFNγ (FIG. 29E), TNFα (FIG. 29F), and Granzyme B (FIG. 29G) in TILs and conditioned medium. qPCR assay also measured relative expression for exhaustion marker PD-1 (FIG. 29H), TIM-3 (FIG. 29I), LAG3 (FIG. 29J), and TIGIT (FIG. 29K).

FIGS. 30A-30J depict the efficacy of Neo-TILs™ in pancreatic cancer. TIL infiltration was measured in a pancreatic cancer patient's tumor organoids (FIG. 30A). qPCR assay measured relative expression of IFNγ (FIG. 30B) and Granzyme B (FIG. 30C) in TILs and conditioned medium. MSD assay measured levels of IFNγ (FIG. 30D), TNFα (FIG. 30E), and Granzyme B (FIG. 30F) in TILs and conditioned medium. qPCR assay also measured relative expression for exhaustion marker PD-1 (FIG. 30G), TIM-3 (FIG. 30H), LAG3 (FIG. 30I), and TIGIT (FIG. 30J).

FIGS. 31A-31C depict the efficacy estimate of Neo-TILs™ in time. Neo-TILs™ efficacy in the immune infiltration assay performed on patient tumoroids is maintained after several days relative to conventional TILs. 48 h assay (FIG. 31A), 72 h assay (FIG. 31B), and 120 h assay (FIG. 31C).

FIG. 32 depicts how Neo-TILs recognized selectively tumor tissue from the original patient relative to conventional TILs. Untreated TILs and IL-2 treated TILS and Neo-TILS educated from cancer patient 34 were incubated with cancer patient 24 organoids. Levels of TILs infiltration was analyzed at 24 h, 48 h, and 72 h.

FIG. 33 depicts single cell sequencing of Neo-TILs after peptide education and the rapid expansion protocol (REP). 95% of cells were CD45 positive, 97% were CD3D positive, 98% were CD3E positive, and 80% were CD3G positive.

### DETAILED DESCRIPTION

As used herein and in the appended claims, the singular forms “a,” “an,” and “the” include plural referents unless the context clearly dictates otherwise. For example, “a” or “an” means “at least one” or “one or more.” Thus, reference to “an antibody or antigen binding fragment thereof” refers to one or more antibodies or antigen binding fragments thereof, and reference to “the method” includes reference to equivalent steps and methods disclosed herein and/or known to those skilled in the art, and so forth.

By “tumor infiltrating lymphocytes” or “TILs” herein is meant a population of cells originally obtained as white blood cells that have left the bloodstream of a subject and migrated into a tumor. TILs include, but are not limited to, CD8⁺ cytotoxic T cells (lymphocytes), Th1 and Th17 CD4⁺ T cells, natural killer cells, dendritic cells and M1 macrophages. TILs include both primary and secondary TILs. “Primary TILs” are those that are obtained from patient tissue samples as outlined herein (sometimes referred to as “freshly harvested”), and “secondary TILs” are any TIL cell populations that have been expanded or proliferated.

TILs can generally be defined either biochemically, using cell surface markers, or functionally, by their ability to infiltrate tumors and effect treatment. TILs can be generally categorized by expressing one or more of the following biomarkers: CD4, CD8, TCR alpha/beta, CD27, CD28, CD56, CCR7, CD45Ra, CD95, PD-1, and CD25. Additionally, and alternatively, TILs can be functionally defined by their ability to infiltrate solid tumors upon reintroduction into a patient. TILS may further be characterized by potency—for example, TILS may be considered potent if, for example, interferon (IFN) release is greater than about 50 pg/mL, greater than about 100 pg/mL, greater than about 150 pg/mL, or greater than about 200 pg/mL.

A “neoepitope” is understood in the art to refer to an epitope that emerges or develops in a subject after exposure to or occurrence of a particular event (e.g., development or progression of a particular disease, disorder or condition, e.g., infection, cancer, stage of cancer, etc.). As used herein, a neoepitope is one whose presence and/or level is correlated with exposure to or occurrence of the event. In some embodiments, a neoepitope is one that triggers an immune response against cells that express it (e.g., at a relevant level). In some embodiments, a neoepitope is one that triggers an immune response that kills or otherwise destroys cells that express it (e.g., at a relevant level). In some embodiments, a relevant event that triggers a neoepitope is or comprises somatic mutation in a cell. In some embodiments, a neoepitope is not expressed in non-cancer cells to a level and/or in a manner that triggers and/or supports an immune response (e.g., an immune response sufficient to target cancer cells expressing the neoepitope). In some embodiments, a neoepitope is a neoantigen.

As used herein, the term “neoantigen-specific tumor-infiltrating lymphocyte” (also shown as Neo-TILs™) refers to the tumor-infiltrating lymphocytes (TILs) produced according to the method described herein.

The terms “target,” “target molecule,” and “target agent” are used interchangeably herein and refer to a protein, toxin, enzyme, pathogen, cell or biomarker that is incubated with a library to identify peptides demonstrating specific binding to the target.

The terms “peptide”, “polypeptide,” and the like are used interchangeably herein, and refer to a polymeric form of amino acids of any length, chemically or biochemically modified or derivatized amino acids, and polypeptides having modified peptide backbones.

The term “peptide construct” as used herein, refers to a peptide of any length attached to an identifying oligonucleotide. The attachment may be via an intervening linker and the attachment may be covalent or non-covalent. The identifying oligonucleotide may be the message that was translated to form the peptide portion of the construct, or it may be any other sequence that is known and can be used to identify the attached peptide by sequencing. ‘Peptide construct sets’ refer to a pool of peptide constructs generated from a custom-designed set of oligonucleotides. The sets may contain as few as one copy per species of peptide construct but typically contain many copies of each peptide construct.

As used herein, the term “binding” refers to an attractive interaction between two molecules which results in a stable association in which the molecules are in close proximity to each other. Molecular binding can be classified into the following types: non-covalent, reversible covalent and irreversible covalent. Molecules that can participate in molecular binding include proteins, nucleic acids, carbohydrates, lipids, and small organic molecules such as pharmaceutical compounds. For example, proteins that form stable complexes with other molecules are often referred to as receptors while their binding partners are called ligands. Nucleic acids can also form stable complex with themselves or others, for example, DNA-protein complex, DNA-DNA complex, DNA-RNA complex.

As used herein, the term “specific binding” refers to the specificity of a binder, e.g., an antibody, such that it preferentially binds to a target, such as a polypeptide antigen. When referring to a binding partner, e.g., protein, nucleic acid, antibody, or other affinity capture agent, etc., “specific binding” can include a binding reaction of two or more binding partners with high affinity and/or complementarity to ensure selective hybridization under designated assay conditions. Typically, specific binding will be at least three times the standard deviation of the background signal. Thus, under designated conditions the binding partner binds to its particular target molecule and does not bind in a significant amount to other molecules present in the sample. Recognition by a binder or an antibody of a particular target in the presence of other potential interfering substances is one characteristic of such binding. Preferably, binders, antibodies or antibody fragments that are specific for or bind specifically to a target bind to the target with higher affinity than binding to other non-target substances. Also preferably, binders, antibodies or antibody fragments that are specific for or bind specifically to a target avoid binding to a significant percentage of non-target substances, e.g., non-target substances present in a testing sample. In some embodiments, binders, antibodies, or antibody fragments of the present disclosure avoid binding greater than about 90% of non-target substances, although higher percentages are clearly contemplated and preferred. For example, binders, antibodies, or antibody fragments of the present disclosure avoid binding about 91%, about 92%, about 93%, about 94%, about 95%, about 96%, about 97%, about 98%, about 99%, and about 99% or more of non-target substances. In other embodiments, binders, antibodies, or antibody fragments of the present disclosure avoid binding greater than about 10%, 20%, 30%, 40%, 50%, 60%, or 70%, or greater than about 75%, or greater than about 80%, or greater than about 85% of non-target substances.

The terms “capture agent” and “capture group” as used herein refer to any moiety that allows capture of a target molecule or a peptide construct via binding to or linkage with an affinity group or domain on the target molecule or an affinity tag of the peptide construct. The binding between the capture agent and its affinity tag may be a covalent bond and/or a non-covalent bond. A capture agent includes, e.g., a member of a binding pair that selectively binds to an affinity tag on a fusion peptide, a chemical linkage that is added by recombinant technology or other mechanisms, co-factors for enzymes and the like. Capture agents can be associated with a peptide construct using conventional techniques including hybridization, cross-linking (e.g., covalent immobilization using a furocoumarin such as psoralen), ligation, attachment via chemically-reactive groups, introduction through post-translational modification and the like.

“Sequence determination,” “sequencing,” and the like include determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput sequencing” or “next generation sequencing” includes sequence determination using methods that determine many (typically thousands to billions) of nucleic acid sequences in an intrinsically parallel manner, i.e., where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeg™ technology by Illumina, Inc., San Diego, Calif.; HeliScope™ by Helicos Biosciences Corporation, Cambridge, Mass.; and PacBio RS by Pacific Biosciences of California, Inc., Menlo Park, Calif.), sequencing by ion detection technologies (such as Ion Torrent™ technology, Life Technologies, Carlsbad, Calif.); sequencing of DNA nanoballs (Complete Genomics, Inc., Mountain View, Calif.); nanopore -based sequencing technologies (for example, as developed by Oxford Nanopore Technologies, LTD, Oxford, UK), and like highly parallelized sequencing methods.

The term “exome” is used in accordance with its art-understood meaning referring to the set of exon sequences that are found in a particular genome.

As used herein, the term “mutation” refers to permanent change in the DNA sequence that makes up a gene. In some embodiments, mutations range in size from a single DNA building block (DNA base) to a large segment of a chromosome. In some embodiments, mutations can include missense mutations, frameshift mutations, duplications, insertions, nonsense mutation, deletions, and repeat expansions. In some embodiments, a missense mutation is a change in one DNA base pair that results in the substitution of one amino acid for another in the protein made by a gene. In some embodiments, a nonsense mutation is also a change in one DNA base pair. Instead of substituting one amino acid for another, however, the altered DNA sequence prematurely signals the cell to stop building a protein. In some embodiments, an insertion changes the number of DNA bases in a gene by adding a piece of DNA. In some embodiments, a deletion changes the number of DNA bases by removing a piece of DNA. In some embodiments, small deletions may remove one or a few base pairs within a gene, while larger deletions can remove an entire gene or several neighboring genes. In some embodiments, a duplication consists of a piece of DNA that is abnormally copied one or more times. In some embodiments, frameshift mutations occur when the addition or loss of DNA bases changes a gene's reading frame. A reading frame consists of groups of 3 bases that each code for one amino acid. In some embodiments, a frameshift mutation shifts the grouping of these bases and changes the code for amino acids. In some embodiments, insertions, deletions, and duplications can all be frameshift mutations. In some embodiments, a repeat expansion is another type of mutation. In some embodiments, nucleotide repeats are short DNA sequences that are repeated a number of times in a row. For example, a trinucleotide repeat is made up of 3-base-pair sequences, and a tetranucleotide repeat is made up of 4-base-pair sequences. In some embodiments, a repeat expansion is a mutation that increases the number of times that the short DNA sequence is repeated.

“Small molecule,” as used herein, means a molecule less than 5 kilodaltons, more typically, less than 1 kilodalton. As used herein, “small molecule” includes peptides.

“Affinity tag” is given its ordinary meaning in the art. An affinity tag is any biological or chemical material that can readily be attached to a target biological or chemical material. Affinity tags may be attached to a target biological or chemical molecule by any suitable method. For example, in some embodiments, the affinity tag may be attached to the target molecule using genetic methods. For example, the nucleic acid sequence coding the affinity tag may be inserted near a sequence that encodes a biological molecule; the sequence may be positioned anywhere within the nucleic acid that enables the affinity tag to be expressed with the biological molecule, for example, within, adjacent to, or nearby. In other embodiments, the affinity tag may also be attached to the target biological or chemical molecule after the molecule has been produced (e.g., expressed or synthesized). As one example, an affinity tag such as biotin may be chemically coupled, for instance covalently, to a target protein or peptide to facilitate the binding of the target to streptavidin.

Affinity tags include, for example, metal binding tags such as histidine tags, GST (in glutathione/GST binding), streptavidin (in biotin/streptavidin binding). Other affinity tags include Myc or Max in a Myc/Max pair, or polyamino acids, such as polyhistidines. At various locations herein, specific affinity tags are described in connection with binding interactions. The molecule that the affinity tag interacts with (i.e., binds to), which may be a known biological or chemical binding partner, is the “recognition entity.” It is to be understood that the invention involves, in any embodiment employing an affinity tag, a series of individual embodiments each involving selection of any of the affinity tags described herein.

A “recognition entity” may be any chemical or biological material that is able to bind to an affinity tag. A recognition entity may be, for example, a small molecule such as maltose (which binds to MBP, or maltose binding protein), glutathione, NTA/Ni²⁺, biotin (which may bind to streptavidin), or an antibody. An affinity tag/recognition entity interaction may facilitate attachment of the target molecule, for example, to another biological or chemical material, or to a substrate (e.g., a nitrocellulose membrane or other immobilized substrate). Examples of affinity tag/recognition entity interactions include polyhistidine/NTA/Ni²⁺, glutathione S-transferase/glutathione, maltose binding protein/maltose, streptavidin/biotin, biotin/streptavidin, antigen (or antigen fragment)/antibody (or antibody fragment), and the like.

The term “ribosomal display” refers to a reaction system able to yield a ternary complex of an mRNA, ribosome and corresponding protein of interest. Ribosomal display can be used for screening cell surface receptors, antibodies, and fragments thereof for target antigen or ligand binding. The steps of producing the reaction system can include: 1) generating a DNA library and transcribing the library into an RNA library, 2) purifying the RNA and in vitro translation in a cell-free protein synthesis system, 3) allowing the ribosome complexes of the translation reaction to bind to a target antigen or ligand, 4) selecting bound ribosome complexes; and 5) isolating RNA from the complexes and reverse transcribing the transcripts to cDNA, wherein the cDNA can be amplified, sequenced and/or further modified.

As used herein, the terms “administration” and “administering” of an agent to a subject include any route of introducing or delivering the agent to a subject to perform its intended function. Administration can be carried out by any suitable route, including intravenously, intramuscularly, intraperitoneally, or subcutaneously. Administration includes self-administration and the administration by another.

The term “effective amount” or “therapeutically effective amount” refers to that amount of an agent or combination of agents as described herein that is sufficient to affect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells. The specific dose will vary depending on the particular agents chosen, the dosing regimen to be followed, whether the agent is administered in combination with other agents, timing of administration, the tissue to which it is administered, and the physical delivery system in which the compound is carried.

The terms “treatment,” “treating,” “treat,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine.

As used herein, the term “patient” or “subject” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes cancer, or presence of one or more tumors. In some embodiments, the disorder or condition is metastatic cancer.

Described herein are methods for producing therapeutic tumor-infiltrating lymphocytes (TILs). TILs therapy is an autologous cellular immunotherapy in which TILs are isolated from surgically-resected tumor specimens, expanded in vitro, and then reinfused into the patient after a lymphodepleting conditioning regimen. While traditional TIL therapy has achieved some impressive responses against a range of cancers, its success remains sporadic. However, there are reasons to think that TIL therapy can be a broadly applicable therapeutic. First, it is known that the majority of tumors are infiltrated by neoantigen-specific T cells, albeit sometimes at very low frequencies. Second, it has been shown that the expansion and infusion of a highly pure TIL product comprising CD4 or CD8 T cells reactive to a single neoantigen is sufficient to mediate tumor regression.

### Overview of Neoantigen-Specific TIL Processing

A failure to consistently enrich neoantigen-specific cells is likely responsible for the limited success of current TIL therapies. For example, vigorous non-specific in vitro expansion distorts the T cell representation profile and often causes the desired target T cells to be outcompeted by cells of other specificities, particularly to the extent that tumor antigen-specific responses are dominated by exhausted or regulatory phenotypes.

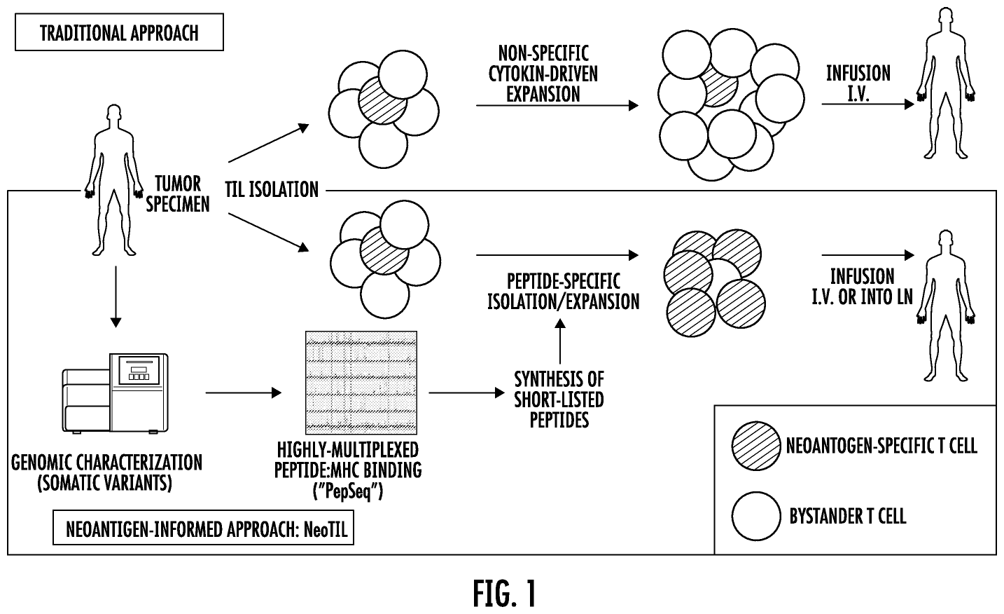

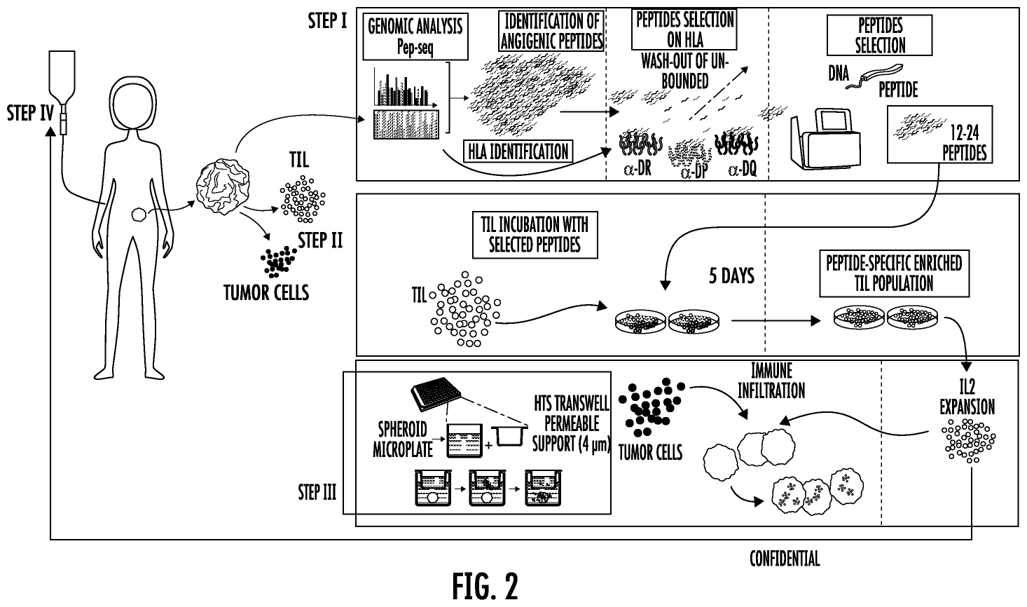

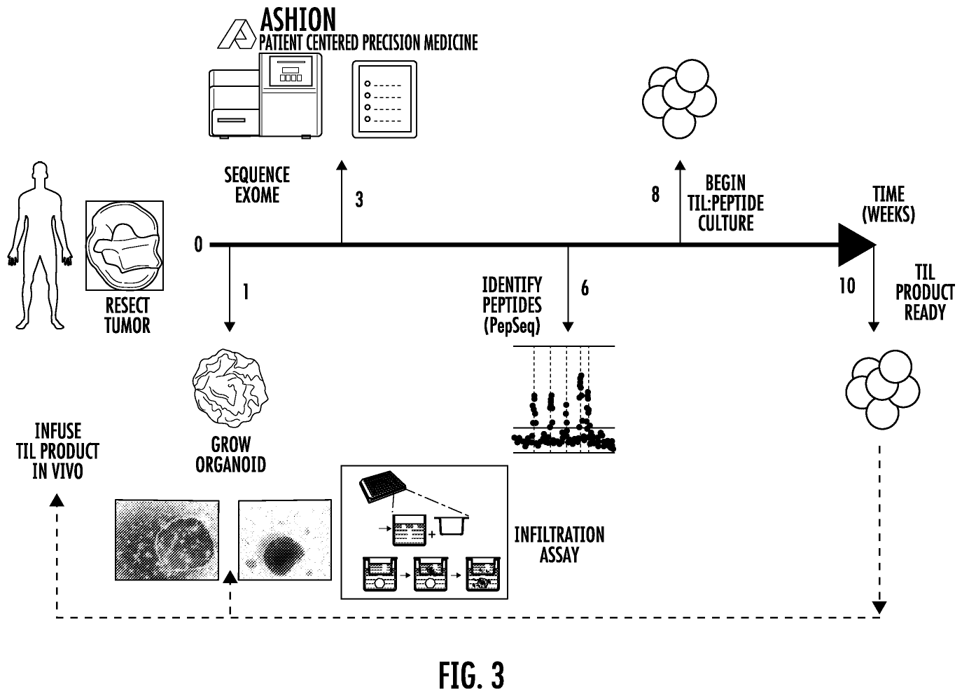

The method described herein is a differentiated approach (FIGS. 1 and 2) in which a novel peptide:MHC screening technology or related methodology is applied to predict personalized immunogenic neoantigens from genomic analysis, and then these neoantigens are used to enrich TIL preparations for the most potent cells, which as a group are referenced herein as neoantigen-specific TILs. Such personalized TIL therapy can be produced in a relatively short timeline (see FIGS. 3 and 4). Also described herein are methods for growing tumor organoids that provide a personalized testbed for the anti-tumor efficacy of the neoantigen-specific TILs.

In some embodiments, the present invention provides a therapeutic approach in which neoantigen-specific T cells are enriched from TILs using one or more of the following non-mutually-exclusive strategies:

(1) Cell sorting based on surface marker expression: In particular, it is known that tumor-antigen specific T cells reside in an exhausted compartment, characterized by high expression of markers including PD1, TIM-3, LAG-3, CTLA-4⁹. In addition, neoantigen specific cells may be identified using surface staining for peptide: MHC multimers.

(2) Antigen-specific expansion using predicted neoantigen peptides: In addition to ‘subtractive’ sorting, neoantigen-specific cells can be enriched by expansion. By culturing TILs in the presence of predicted neoantigenic peptides, it is possible to preferentially expand cells of the desired specificity.

In both of these arms, a rate-limiting step has traditionally been the need to identify the small set of targeted neoantigens from among the typically large catalog of peptide-changing somatic tumor variants. To address this limitation, a powerful approach is deployed in which a novel highly-multiplexed peptide:MHC class II binding assay ('MHC-PepSeq') is paired with in silico predictions for MHC class I. This enables the rapid, high-confidence identification of candidate neoantigen peptide:MHCs for the construction of neoantigen-specific fluorescent labels and/or stimulatory antigens. Alternatively, phage display or a related method is used to identify candidate neoantigen peptide:MHCs.

(3) Injection of minimally expanded TILs through neoantigen peptide exposure into lymph nodes for processing and expansion: It is known that cytokine exposures ex-vivo may change the neoantigen repertoire and T-cell dynamics. In one aspect, the minimally expanded TILs are injected directly into the lymph node areas that preserve the ‘natural’ processing and expansion of these T -cells. This approach is entirely novel because it uses in vivo processing for the TILs.

Upon T cell enrichment, it is possible to use single cell sequencing, potentially in conjunction with peptide: MHC multimer labeling, to identify the paired rearranged TCRα and β genes that confer T cell neoantigen specificity. This can provide an additional metric of the efficiency of T cell enrichment, and also enable a parallel gene transfer approach in which patient-specific neoantigen reactivity is conferred on universal donor cells, offering the potential to circumvent the exhausted phenotype of the endogenous response.

Detection of Tumor Mutations and/or Neoepitopes

Autologous TILs may be obtained from the stroma of resected tumors. For this, tumor samples are obtained from patients and a single cell suspension is obtained. The single cell suspension can be obtained in any suitable manner, e.g., mechanically (disaggregating the tumor using, e.g., a gentleMACSTM Dissociator, Miltenyi Biotec, Auburn, Calif) and/or enzymatically (e.g., collagenase or DNase).

Cancers may be screened to detect mutations and/or neoepitopes (e.g., to detect neoantigen identity, and/or neoepitope nature, level, and/or frequency) as described herein using any of a variety of known technologies. In some embodiments, particular mutations or neoepitopes, or expression thereof, is/are detected at the nucleic acid level (e.g., in DNA or RNA). One of skill in the art would recognize that mutations or neoepitopes, or expression thereof, can be detected in a sample comprising DNA or RNA from cancer cells. Further, one of skill in the art would understand that a sample comprising DNA or RNA from cancer cells can include but is not limited to circulating tumor DNA (ctDNA), cell free DNA (cfDNA), cells, tissues, or organs. In some embodiments, mutations or neoepitopes, or expression thereof, is detected at the protein level (e.g., in a sample comprising polypeptides from cancer cells, which sample may be or comprise polypeptide complexes or other higher order structures including but not limited to cells, tissues, or organs).

In some particular embodiments, detection involves nucleic acid sequencing. In some embodiments, detection involves whole exome sequencing. In some embodiments, detection involves an immunoassay. In some embodiments, detection involves use of a microarray. In some embodiments, detection involves massively parallel exome sequencing. In some embodiments, detection involves genome sequencing. In some embodiments, detection involves RNA sequencing. In some embodiments, detection involves standard DNA or RNA sequencing. In some embodiments, detection involves mass spectrometry.

In some embodiments, detection involves next generation sequencing (DNA and/or RNA). In some embodiments, detection involves genome sequencing, genome resequencing, targeted sequencing panels, transcriptome profiling (RNA-Seq), DNA-protein interactions (ChIP-sequencing), and/or epigenome characterization. In some embodiments, re-sequencing of a patient's genome may be utilized, for example to detect genomic variations.

In some embodiments, detection involves using a technique such as ELISA, western blotting, immunoassay, mass spectrometry, microarray analysis, etc.

In some embodiments, detection involves next generation sequencing (DNA and/or RNA). In some embodiments, detection involves next generation sequencing of targeted gene panels (e.g. the ASHION® tumor/normal exome-RNA test (GEMEXTRA®), MSK-IMPACT, or FOUNDATIONONE®). In some embodiments, detection involves genomic profiling.

In some embodiments, detection involves genomic profiling using the GEMEXTRA® test. The GEMEXTRA® test is a comprehensive exome and transcriptome profiling assay that both informs the care of cancer patients and enables future research into the disease. Greater than 19,000 genes are assayed via hybridization capture and clinical-depth sequencing for the identification of somatic point mutations, small and large insertions and deletions and structural rearrangements. Measures of microsatellite instability (MSI) and tumor mutational burden (TMB) are also taken to inform the application of immunoncology therapies. To ensure somatic origin of identified variants, both the germline and tumor exomes are sequenced and compared. The entire transcriptome is also sequenced, enabling the detection of gene fusion and alternative splicing events from the patient's RNA. GEMEXTRA® is applicable to both solid and hematological cancers.

In some embodiments, detection involves genomic profiling using Integrated Mutation Profiling of Actionable Cancer Targets (MSK-IMPACT) (see Cheng D T, Mitchell T N, Zehir A, et al. Memorial Sloan Kettering-Integrated Mutation Profiling of Actionable Cancer Targets (MSK-IMPACT): A Hybridization Capture-Based Next-Generation Sequencing Clinical Assay for Solid Tumor Molecular Oncology. J Mol Diagn. 2015; 17(3):251-264; and Ross D S, Zehir A, Cheng D T, et al. Next-Generation Assessment of Human Epidermal Growth Factor Receptor 2 (ERBB2) Amplification Status: Clinical Validation in the Context of a Hybrid Capture-Based, Comprehensive Solid Tumor Genomic Profiling Assay. J Mol Diagn. 2017; 19(2):244-254). MSK-IMPACT is a comprehensive molecular profiling assay that involves hybridization capture and deep sequencing of all exons and selected introns of multiple oncogenes and tumor-suppressor genes, allowing for detection of point mutations, small and large insertions or deletions, and rearrangements. MSK-IMPACT also captures intergenic and intronic single-nucleotide polymorphisms (e.g., tiling probes), interspersed across a genome, aiding in accurate assessment of genome-wide copy number. In some embodiments, probes may target a megabase.

In some embodiments, detection involves genomic profiling using the FOUNDATIONONE® CDX^{1M} (“FlCDx”) assay. The FlCDx assay is a next generation sequencing based in vitro diagnostic device for detection of substitutions, insertion, and deletion alterations (indels), and copy number alterations (CNAs) in 324 genes and select gene rearrangements, as well as genomic signatures including microsatellite instability (MSI) and tumor mutation burden (TMB) using DNA isolated from formalin-fixed paraffin embedded (FFPE) tumor tissue specimens. FlCDx is approved by the United States Food and Drug Administration (FDA) for several tumor indications, including NSCLC, melanoma, breast cancer, colorectal cancer, and ovarian cancer.

The FlCDx assay employs a single DNA extraction method from routine FFPE biopsy or surgical resection specimens, 50-1000 ng of which will undergo whole-genome shotgun library construction and hybridization-based capture of all coding exons from 309 cancer-related genes, one promoter region, one non-coding (ncRNA), and selected intronic regions from 34 commonly rearranged genes, 21 of which also include the coding exons. In total, the assay detects alterations in a total of 324 genes. Using the ILLUMINA® HiSeq 4000 platform, hybrid capture-selected libraries are sequenced to high uniform depth (targeting >500×median coverage with >99% of exons at coverage >100×). Sequence data is then processed using a customized analysis pipeline designed to detect all classes of genomic alterations, including base substitutions, indels, copy number alterations (amplifications and homozygous gene deletions), and selected genomic rearrangements (e.g., gene fusions). Additionally, genomic signatures including microsatellite instability (MSI) and tumor mutation burden (TMB) are reported.

In some embodiments, detection may involve sequencing of exon and/or intron sequences from at least 100, 150, 200, 250, 300, 350, 400, 450, 500, 550, 600, 650, 700, 750, 800, 850, 900, 950, 1000 or more genes (e.g., oncogenes and/or tumor-suppressor genes). For example, literature reports indicate that MSK-IMPACT has been used to achieve deep sequencing of all exons and selected introns of 468 oncogenes and tumor-suppressor genes.

Alternatively or additionally, in some embodiments, detection may involve sequencing of intergenic and/or intronic single-nucleotide polymorphisms. For example, literature reports indicate that MSK-IMPACT has been used to achieve deep sequencing of >1000 intergenic and intronic single-nucleotide polymorphisms.

In some embodiments, detection involves sequencing of splice variants. Cancer cells are able to adapt and evolve by developing mechanisms to escape control by their microenvironment. The diversity and plasticity offered by alternative splicing, therefore, provide an opportunity for cancer cells to produce protein isoforms suitable for tumor growth and/or spreading (David C J and Manley J L. Genes Dev. 2010; 24(21):2343-2364). Genome-wide approaches has revealed that large-scale alternative splicing occurs during tumorigenesis (Venables J P et al. Nat Struct Mol Biol. 2009; 16(6):670-676.), and the genomic portraits of alternative splicing patterns have proven useful in the classification of tumors (Venables J P. Bioessays. 2006; 28(4):378-386; Skotheim R I et al., Int J Biochem Cell Biol. 2007; 39(7-8):1432-1449; Omenn G S et al., Dis Markers. 2010; 28(4):241-251).

Reports of aberrant splicing events and alterations in ratios of alternatively spliced transcripts in different cancers have been noted (Raj an P et al., Nat Rev Urol. 2009; 6(8):454-460). These events result in novel transcripts not observed in normal cell counterparts. It has been reported that nearly all areas of tumor biology are affected by alternative splicing, including metabolism, apoptosis, cell cycle control, invasion, metastasis and angiogenesis (Venables J P., Bioessays. 2006; 28(4):378-386; Ghigna C et al., Curr Genomics. 2008; 9(8):556-570).

One of the earliest examples of alternative splice variants with opposing apoptotic effects is Bcl-x. The Bcl-x pre-mRNA can be alternatively spliced to produce two splice variants, anti-apoptotic Bc1-xL (long form) and pro-apoptotic Bc1-xS (short form) (Boise L H et al. Cell. 1993; 74(4):597-608). High Bc1-xL/Bc1-xS ratios, promoting tumor cell survival, can be found in a number of cancer types, including human lymphoma, breast cancer, and human hepatocellular carcinoma (Minn A J et al., J Biol Chem. 1996; 271(11):6306-6312.44-46; Olopade O I et al., Cancer J Sci Am. 1997; 3(4):230-237; Takehara T et al., Hepatology. 2001; 34(1):55-61). Another example of an apoptosis-related gene that undergoes alternative splicing in cancer cells is the Fas receptor gene.

Expressed on the cell surface of many cell types, the Fas receptor is activated by the Fas ligand produced by cytotoxic T cells, which initiates a death-signaling cascade leading to apoptosis of cells expressing the Fas receptor (Bouillet P et al, Nat Rev Immunol. 2009; 9(7):514-519). There are at least 3 short mRNA variants of Fas missing the encoded transmembrane domain and the resulting translated protein variants are presumably secreted by cancer cells and act as decoy receptors for the Fas ligand, thus allowing cancer cells to escape from apoptosis (Cheng J et al., Science. 1994; 263(5154):1759-1762; Cascino I et al., Journal of immunology. 1995; 154(6):2706-2713).

Alternative splicing of the H-Ras oncogene occurs on a previously unknown spliced exon (named as IDX) caused by an intronic mutation in the H-Ras gene (Cohen J B et al., Cell. 1989; 58(3):461-472). This mutation of the IDX splice site results in an H-Ras mRNA variant, which is more resistant to the nonsense-mediated mRNA decay (NMD) process, and consequently overexpressed in cancers (Barbier J et al., Mol Cell Biol. 2007; 27(20):7315-7333). Alternative splicing also plays a role in promoting invasive and metastatic behavior in cancers. CD44 was among the first genes with splice variants specifically associated with metastasis, where variants containing exons 4-7 (v4-7) and 6-7 (v6-7) were shown to be expressed in a metastasizing pancreatic carcinoma cell line, but not in the corresponding parental tumor (Gunthert U et al., Cell. 1991; 65(1):13-24).

Mutational Calling Software Tools In certain aspects, a Pegasus human research pipeline is used. The sequences are aligned to the HG19 human genome or the HG38 human genome. In one aspect, somatic mutations are called using three separate mutational callers: Seurat, Mutect, and Strelka. All mutations that were seen in more than one of the callers were selected and used to generate peptides. The rationale behind this is that each caller has its own strengths and weaknesses. By pulling mutations that are called by multiple callers the risk of identifying false positives is reduced. Best practices are followed for DNA and RNA workflows. Alternatively, CLIA certified somatic calls are used with no changes to generate peptides as candidate neoantigens for further analysis.

In some embodiments, single or multiple mutation callers may be utilized to call genomic variants. For samples consisting of sequencing tumor and normal tissue the callers that may be used are: Strelka, Strelka2, VarDict, VarScan2, qSNP, Shimmer, RADIA, SOAPsnv, SomaticSniper, FaSD-somatic, Samtools, JointSNVMix, Virmid, SNVSniffer, Seurat, CaVEMan, MuTect, MuTect2, LoFreq, EBCall, deepSNV, LoLoPicker, MuSE, MutationSeq, SomaticSeq, SnooPer, FreeBayes, HapMuC, SPLINTER, Pisces, DeepSNVMiner, smCounter, DeepVariant, Cake, Tnscope, DNAscope, NeoMutate, Maftools, scABA, Sanivar, Sarek, BATCAVE, SomaticNet, CoVaCS, RegTools, xAtlas, R2D2, SiNPle, Bambino, and exactSNP. For samples consisting of tumor only sequencing callers that may be utilized are: Platypus, LumosVar, LumosVar2, SNVMix2, SNVer, OutLyzer, ISOWN, SomVarIUS, SiNVICT, FreeBayes, SNPiR, eSNV-dectect, RNAIndel, VaDir, and Clairvoyante.

In certain embodiments, single or multiple mutation callers recognizing splice variants may be utilized to call genomic variants. For samples consisting of sequencing tumor and normal tissue the callers that may be used are: RegTools, ASGAL, MATS (rMATS), SUPPA (SUPPA2), Leafcutter, MAJIQ, JunctionSeq, findAS, Cufflinks/Cuffdiff, IsoformSwitchAnalyzeR, ALEXA-seq, MISO, SplicingCompass, Flux Capacitor, JuncBASE, SpliceR, FineSplice, ARH-seq, Spladder, DEXSeq, edgeR, Limma, DiffSplice, dSpliceType, SpliceDetector, HISAT2, STAR, Subread, Subjunct, PennDiff, DSGseq, AltAnalyze, Splicing Express, SpliceTrap, PSGInfer, FDM, IsoEM2, MADS+, Spanki, SpliceMap, CASPER, AVISPA, PASA, SpliceSeq, MATT, Aspli, IPSA, SANJUAN, VAST-Tools, SpliceV, Asprofile, DESeq2, Yanagi, ABLas, IRIS, and AS-Quant.

### HLA Typing

In some embodiments, whole exome sequencing (WES) and/or RNA sequencing from tumor and/or normal tissue may be used to identify the MHC class 1 and 2 genotypes for individual patients.

Single or multiple HLA callers may be utilized to identify the HLA types. For DNA sequencing the HLA callers that may be utilized are: BWAkit, PHLAT, OptiType, HLAMiner, HISAT, HISAT2, xHLA, HLA-Vbseq, GATK HLA Caller, PolySolver, HLAReporter, Athlates, HLAseq, HLAssign, SOAP-HLA, STC-SEq, HapLogic, Gyper, HLATyphon, HLA-LA, HLA-PRG, MHC-PRG, Prohlatype, GraphTyper, ALPHLARD, Omixon, SNP2HLA, HLAscan, NeoEpitopePred, and Assign. For RNA sequencing the HLA callers that may be utilized are: BWAkit, seq2HLA, PHLAT, HLAProfiler, OptiType, HLAMiner, HLAForest, arcasHLA, HISAT, HISAT2, HLAseq, HLAssign, STC-Seq, HLATyphon, ALPHLARD, Omixon, and HLAscan.

In some embodiments, whole exome sequencing data may be analyzed for HLA typing using Polysolver (polymorphic loci resolver) (Shukla et al., 2015). Polysolver is an algorithm for inferring alleles of the three major MHC class I (HLA-A, HLA-B, HLA-C) genes. For example, a patient's HLA class I alleles may be inferred from whole exome sequencing data using Polysolver. In other variations, in the absence of whole exome sequencing data, a patient's HLA class I alleles may be inferred from transcriptome sequencing data by an alternative HLA typing algorithm.

In one embodiment, HLA typing is conducted in silico using one or more techniques such as OptiType, run on a computing device. See Szolek, et al., OptiType: precision HLA typing from next-generation sequencing data, Bioinformatics. 2014 Dec 1;30(23), incorporated herein in its entirety for all purposes. A variety of other in silico techniques may also be used. See Major, et al., HLA typing from 1000 genomes whole genome and whole exome Ilumina data, PLoS One. 2013 Nov. 6; 8(1 1):e78410; Wittig, et al., Development of a high-resolution NGS-based HLA-typing and analysis pipeline, Nucl. Acids Res. (2015).

Generation of Peptides from Tumor Mutations

In some embodiments mutational data is acquired as described above in the section entitled “Detection of Tumor Mutations and/or Neoepitopes”. The mutational calls may be converted to mutant protein sequences using single or multiple tools including but not limited to: Varcode, customProDB, and pyGeno. The tools will generate the peptides sequences for downstream in-vitro and in-silico HLA binding assays. Additionally, in some embodiments, the peptides will undergo testing to validate that each peptide is unique to the human proteome. The peptides may be aligned to several different protein databases from NCBI including: Non-redundant protein sequences, Reference proteins, model organisms, UniProtKB/Swiss-Prot, Patented protein sequences, Protein Data Bank proteins, Metagenomic proteins, and Transcriptome Shotgun Assembly proteins. Using the databases listed above rBlast may be utilized to run protein BLAST to identify proteins with 100% identity to the human proteome.

In certain aspects, Varcode software is utilized in python to predict the impact of the genome variant data. Varcode generates the wild type and mutant protein sequences from the mutation and/or neoepitopes at the desired peptide size. In certain aspects, the desired peptide size is 3, 4, 5, 6, 7, 8, 9, 10, 11, 12, 13, 14, 15, 16, 17, 18, 19, 20 or more amino acids. In one aspect, the desired peptide size is 15 amino acids.

Varcode dependency is Pyensembl, which is a python access point to Ensembl reference genome data to generate peptides. In some embodiments, Varcode predicts the impact of each mutation on the protein. Silent mutations and mutations that are in noncoding regions on the genome are excluded. Varcode is then used to generate mutations across the peptide sequence. These variant peptides (i.e., candidate neoantigens) containing the mutations generated with Varcode are further analyzed with in silico methods and/or biochemical assays to identify which peptides bind with TILs from the patient.

### Analysis of Neoantigens

In strategies (1) and (2) identified above, a rate-limiting step has traditionally been the need to identify the small set of targeted neoantigens from among the typically large catalog of peptide-changing somatic tumor variants. The recently developed screening technologies for studying peptide binding can addresses this limitation.

In some embodiments, a novel highly-multiplexed peptide:target protein assay is paired with in silico prediction to identify the neoanitgents. For example, a highly-multiplexed peptide:MHC class II binding assay is paired with in silico predictions for MHC class I is used. This enables the rapid, high-confidence identification of candidate neoantigen peptide:MHCs for the construction of neoantigen-specific fluorescent labels and/or stimulatory antigens. Examples of these highly-multiplexed peptide:target protein binding assay as described in U.S. Pat. Nos. 9,958,454; 10,288,608; U.S. Patent Application Publication No. 2016/0025726; and International Patent Application No. PCT/US2021/013774.

Alternatively, phage display or a related method is used to identify candidate neoantigen peptide:MHCs.

In other embodiments, the polypeptides of interest are genetically encoded to facilitate identification of neoantigens. An example of a genetically encoded polypeptide library is a mRNA display library. Another example is a replicable genetic display package (rgdp) library such as a phage display library. In one embodiment, the polypeptides of interest are genetically encoded as a phage display library. In these embodiments, the nucleic acid may be comprised by the phage genome. In these embodiments, the polypeptide may be comprised by the phage coat.

In some embodiments, the invention may be used to produce a genetically encoded combinatorial library of polypeptides which are generated by translating a number of nucleic acids into corresponding polypeptides and linking molecules of said molecular scaffold to said polypeptides. The genetically encoded combinatorial library of polypeptides may be generated by phage display, yeast display, ribosomal display, bacterial display or mRNA display.

### Highly-Multiplexed Peptide: Target Protein Binding Assay

In some aspects, identification of neoantigen comprises providing a library of peptide constructs, wherein each peptide construct of the library comprises a peptide portion and an identifying nucleic acid portion that identifies the peptide portion, and the peptide portion of at least one of the peptide constructs is capable of specific binding to the HLA proteins or fragments thereof; contacting the HLA proteins or fragments thereof with the library of peptide constructs; separating the at least one peptide construct comprising a peptide portion capable of specific binding to the HLA proteins or fragments thereof from peptide constructs comprising a peptide portion not capable of specific binding to the HLA proteins or fragments thereof; sequencing all or a portion of the identifying nucleic acid portion of the at least one peptide construct capable of specific binding to the HLA proteins or fragments thereof. In some aspects, the identifying nucleic acid portion of each peptide construct comprises a polynucleotide sequence or complement thereof encoding the peptide portion of the peptide construct. In particular implementation, the PepSeq platform is used for the identification of the candidate neoantigens.

PepSeq is a technology that can rapidly generate a large number of potential neoantigens as candidate binding agents for isolated TILs. PepSeq is a method that generates peptide libraries (10-50 amino acids long) with each peptide conjugated to a unique DNA tag that can be used to monitor peptide abundance following binding experiments. Importantly, the peptide sequences can be used in large multiplexed binding assays with upwards of 100,000 of unique programmable peptides. Binding assays for any molecular target can be used to screen large diverse PepSeq libraries. A biological structure (e.g., a population of isolated TILs) can be mixed with diverse peptides, separated from unbound peptides, and then queried for those that “stick.” The particular libraries could be intelligently designed libraries based upon prior knowledge of the target (e.g., from exome sequencing and analysis). The PepSeq platform is described in greater detail in U.S. Pat. Nos. 9,958,454; 10,288,608; and U.S. Patent Application Publication No. 2016/0025726, the contents of which are hereby incorporated by reference.

In particular implementations, the peptide construct identified as being bound to the HLA protein or fragment thereof may be further modified for enhanced binding to the HLA protein or fragment thereof. And it is the peptides with enhanced binding to the HLA protein that are used for culturing the TILs to produce neoantigen-specific TILs. Thus, in some aspects, the identification of the neoantigen further comprises generating a library of peptide constructs based on the peptide construct identified as being bound to the HLA protein or fragment thereof (subsequently referred to as “the first peptide”) and identifying a threshold z-score of the binding of the first peptide and the HLA protein or fragment thereof. The library of peptide constructs comprises a peptide construct comprising the first peptide and a plurality of peptide constructs comprising variant peptides. The method further comprises contacting the HLA protein or fragment thereof with the library of peptide and identifying at least one variant peptide with increased binding to the the HLA protein or fragment thereof compared to that of the first peptide. Increased binding is indicated by a z-score higher than the identified threshold z-score of the first peptide. The z-score of a peptide is calculated by first determining a relative abundance level of each peptide constructs in the library of peptide constructs and then grouping the grouping peptide constructs into bins based on similarity of relative abundance level, wherein each bin comprises at least 300 peptide constructs. The relative abundance level of each peptide construct is also normalized against the average of the relative abundance level of the negative control peptide constructs in the library of peptide constructs. The normalized relative abundance levels of each peptide construct in a bin are used to determine a mean and a standard deviation of each bin. The z-score of a peptide is calculated based on the mean and a standard deviation of its bin. In some aspects, the determination of the mean and the standard deviation of the normalized relative abundance levels in a bin excludes peptide constructs having outlier relative abundance levels. In some aspects, a peptide construct has an outlier relative abundance level when its normalized relative abundance level is outside the 95% highest density interval of its bin. In certain implementations, 5% of peptide constructs in each bin are excluded from the determination of the mean and the standard deviation of the normalized relative abundance levels in a bin.

In some embodiments, the identification of the neoantigen further comprises generating a second library of peptide constructs, contacting the the HLA protein or fragment thereof with the second library of peptide constructs, and identifying at least one variant peptide from the second library of peptide constructs with increased binding to the the HLA protein or fragment thereof compared to that of the second peptide. The second library of peptide constructs is based on a second peptide identified as having increased binding to the the HLA protein or fragment thereof compared to that of the first peptide, for example by having a z-score higher than the threshold z-score of the first pepetide. Thus, the second library of peptide constructs comprises a peptide construct comprising the second peptide and a second plurality of peptide constructs comprising variant peptides. The at least one variant peptide from the second library of peptide constructs with increased binding to the the HLA protein or fragment thereof compared to that of the second peptide has a higher z-score than the z-score of the second peptide.

In some aspects, the variant peptides of the plurality of peptide constructs are produced by complete single residue mutagenesis, sliding window mutagenesis, or by alanine scanning or glycine scanning mutagenesis. In certain implementations, the plurality of variant peptides comprises at least one of the sets of variant peptides produce by complete single residue mutagenesis, sliding window mutagenesis, and alanine scanning mutagenesis. More details of this method finding improved binding targets may be found International Patent Application No. PCT/US2021/013774.

### Phage Display

Different phage display systems have been developed throughout the years, making use of different phage vectors (M13 filamentous phage, lambda, T4, and T7 phage) and various phage coat proteins for covalent fusion. Techniques and methodology for performing phage display can be found in WO 2009/098450. Phage display is described, for example, in Ladner et al., U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem 274:18218-30; Hoogenboom et al. (1998) Immunotechnology 4:1-20; Hoogenboom et al. (2000) Immunol Today 2:371-8; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins et al. (1992) J Mol Biol 226:889-896; Clackson et al. (1991) Nature 352:624-628; Gram et al. (1992) Proc Natl Acad Sci USA 89:3576-3580; Garrard et al. (1991) Bio/Technology 9:1373-1377; Rebar et al. (1996) Methods Enzymol. 267:129-49; Hoogenboom et al. (1991) Nuc Acid Res 19:4133-4137; and Barbas et al. (1991) PNAS 88:7978-7982.

### Ribosomal Display

In certain aspects, the methods described herein comprise the use of ribosomal display to identify neoantigens. Ribosomal display can be used to perform in vitro protein synthesis of a protein of interest (e.g., a candidate neoantigen). Ribosomal display can also be used to perform in vitro protein evolution to create proteins with desired properties, for example, Fab fragments that bind to a specific target molecule. Ribosomal display is a well-known technology useful for generating libraries. This entirely in vitro method allows for libraries with a diversity of 10¹⁶ members. The process results in translated proteins that are associated with their RNA progenitor which can be used, as a complex, to select for proteins with desired properties (e.g., bind to an immobilized target molecule). The RNA-protein complex that shows the desired property, e.g., high binding affinity, can then be reverse transcribed to cDNA and the sequence amplified via PCR. The process also provides for repeated cycles of iteration or protein expression. In addition, nucleic acid mutations can be introduced efficiently into the selected nucleic acid library in subsequent cycles, leading to continuous DNA diversification and thus, protein evolution. The end result is a nucleic acid sequence that can be used to produce a protein of interest (e.g., neoantigens that specifically bind to TILs).

In the method provided herein, a protein of interest is displayed on the surface of a ribosome from which the protein of interest is being translated. Briefly, a library of RNA molecules is translated in an in vitro translation system to the 3′ end of the RNA molecule, such that the ribosome does not fall off. This is accomplished by not incorporating a functional stop codon in the RNA template. Normally, the stop codon is recognized by release factors that trigger detachment of the nascent polypeptide from the ribosome. In ribosomal display, the peptide emerges from the ribosome, but does not fall free of the complex. This allows, for example, the nascent polypeptide to associate with another polypeptide to form a functional dimer. In some instances, there is an additional folding step in an oxidizing environment (important for forming disulfide bonds).

The whole complex of folded protein of interest, ribosome and RNA, which is stable for several days, can then be screened for desired properties, such as specific binding to a binding pair ligand by the translated protein of interest. The RNA encoding the selected protein of interest can be reverse transcribed into single stranded cDNA which can be converted to double-stranded DNA and amplified by PCR, providing the coding sequence of the selected protein (e.g., a neoantigen). The reverse transcription reaction can be performed on mRNA associated in the ribosomal display complex, or the mRNA can be isolated from the ribosomal display complex then used in the reverse transcription step. Suitable methods for disruption/dissociation of ribosome complexes are known in the art and include EDTA treatment and/or phenol extraction.

In general, nucleic acid (DNA) constructs for ribosomal display contain a promoter (T7, SP6 or T3), a translation initiation signal such as a Shine-Dalgarno (prokaryotic) or Kozak (eukaryotic) sequence, initiation codon, and coding sequence of the protein of interest (e.g., V_{H} or V_{L} chain domain). One or more nucleic acid sequences encoding one or more detection tags may be included to provide for production of a protein further comprising one or more detection tags (e.g., histidine tag). To enable the complete nascent protein to be displayed and fold into its active conformation, a spacer domain of at least 23-30 amino acids length can be added at the C terminus, to allow the protein to exit completely from the ribosome. The spacer can provide a known sequence for the design of primers for RT-PCR recovery of the DNA sequences.

To remove the stop codon from DNA, a 3′ primer lacking the stop codon can be used during PCR construction. Constructs designed for bacterial-based display systems can incorporate sequences containing stem-loop structures at the 5′ and 3′ ends of the DNA to stabilize mRNA against degradation by RNase activities in bacterial cell-free systems.

The mRNA translation system used in the methods described herein may be any suitable available system. A prokaryotic or eukaryotic translation system may be used, for example crude E. coli lysate (commercially available from e.g. Promega Corp., Madison, Wis.; Agilent Technologies, Santa Clara, Calif.; GE Healthcare Biosciences, Pittsburgh, Pa.; Life Technologies, Carlsbad, Calif.), a reconstituted ribosome system such as PURE (see, e.g., Shimizu et al., Nat. Biotechnol., 19:751-755 (2001)), or a cell free protein synthesis system as described below.

The PURE system can include about 32 individually purified components used for in vitro protein biosynthesis (e.g., initiation, elongation and termination). In some embodiments, the components include initiation factors (e.g., IF1, IF2, IF3), elongation factors (e.g., EF-G, EF-Tu, EF-Ts), release factors (e.g., RF1, RF3), a termination factor (e.g., RRF), 20 aminoacyl-tRNA synthetases, methionyl-tRNA transformylase, T7 RNA polymerase, ribosomes, 46 tRNAs, NTPs, creatine phosphate, 10-formyl-5,6,7,8-tetrahydrofolic acid, 20 amino acids, creatine kinase, myokinase, nucleoside-diphosphate kinase and pyrophosphatase.

Ribosomal display has been used to successfully generate antibody fragments with high affinity for their target. Detailed description of ribosomal display is found in, e.g., Hanes, J. Proc. Natl. Acad. Sci. USA, 95: 14130-14135 (1998); Schaffitzel et al., J. Immunological Methods, 231:119-135 (1999); He et al., J. Immunological Methods, 231, 105-117 (1999); Roberts R W, Current Opinion in Chemical Biology, 3: 268-273 (1999).

### II. Other Display Methods for In Vitro Selection

Other in vitro library display methods can be used in the methods described herein, such as, but not limited to, mRNA display, bicistronic display, P2A display, and CIS display (cis-activity display).

mRNA Display

In mRNA display, each member of the RNA library is directly attached to the protein of interest it encodes by a stable covalent linkage to puromycin, an antibiotic that can mimic the aminoacyl end of tRNA (see, e.g., Robert R W and Szostak J W, Proc. Natl. Acad. Sci. USA, 94:12297-12302 (1997)). Puromycin is an aminonucleoside antibiotic, active in either prokaryotes or eukaryotes, derived from Streptomyces alboniger. Protein synthesis is inhibited by premature chain termination during translation taking place in the ribosome. Part of the molecule acts as an analog of the 3′ end of a tyrosyl-tRNA, where a part of its structure mimics a molecule of adenosine and another part mimics a molecule of tyrosine. It enters the A site and transfers to the growing chain, causing the formation of a puromycylated nascent chain and premature chain release. The 3′ position contains an amide linkage instead of the normal ester linkage of tRNA, making the molecule much more resistant to hydrolysis and stopping procession along the ribosome.

Other puromycin analog inhibitors of protein synthesis include O-demethylpuromycin, O-propargyl-puromycin, 9-{3′-deoxy-3′-[(4-methyl-L-phenylalanyl)amino]-β-D-ribofuranosyl}-6-(N,N-dimethylamino)purine [L-(4-Me)-Phe-PANS] and 6-dimethylamino-9-[3-(p-azido-L-beta-phenylalanylamino)-3-deoxy-beta-ribofuranosyl]purine.

Members of the RNA library can be ligated to puromycin via a linker such as, but not limited to, a polynucleotide or a chemical linker, e.g., polyethylene glycol (Fukuda et al., Nucleic Acid Research, 34(19): e127 (2006)). In some embodiments, the polynucleotide linker comprising RNA is linked at the 3′ terminal end to puromycin. In other embodiments, the PEG liker is linked to puromycin.

As puromycin that is linked to the 3′ terminal end of a RNA molecule enters a ribosome, it establishes a covalent bond to the nascent protein (encoded by the RNA molecule) as a result of peptidyl transferase activity in the ribosome. In turn, a stable amide linkage forms between the protein and the O-methyl tyrosine portion of puromycin.

The RNA library of RNA-puromycin fusions can undergo in vitro translation, as described herein, to generate RNA-puromycin-protein complexes (e.g., RNA-puromycin-neoantigen complexes). In some embodiments, the RNA-puromycin fusions comprise RNA molecules encoding candidate neoantigens identified by exome analysis of tumor cells and wild-type cells from a patient.

Affinity selection can be performed on the mRNA-displayed protein library to screen for proteins having desired properties, such a specific binding to a binding pair ligand. The mRNA display can be performed in solution or on a solid support. The selected mRNA-displayed protein of interest can be purified by standard methods known in the art such as affinity chromatography. The mRNA can be cloned, PCR amplified, and/or sequenced to determine the coding sequence of the selected protein of interest. In one embodiment, the selected mRNA-displayed library contains a population of neoantigens that bind to TILs.

In some aspects, members of a library of nucleic acid members are linked to puromycin, where each member encodes a prospective protein of interest having a primary amino acid sequence different from the other proteins encoded by the other nucleic acid members. The mRNA display system can contain a population or mixture of different complexes such that each complex has a different mRNA linked to puromycin, the ribosome, and a prospective protein of interest.

### Biscistronic DNA Display

Bicistronic DNA display can be employed for in vitro selection of proteins of interest (see, e.g., Sumida et al., Nucleic Acid Research, 37(22):e147 (2009)). The method is based on complexing in vitro translated proteins of interest to their encoding DNA, which used to determine the sequence of the protein of interest. Typically, a DNA template containing multiple ORFS that can be linked to the protein it encodes is generated. In addition, the coupled transcription/translation reaction is compartmentalized in a water-in-oil emulsion (e.g., micelle).

In some instances, when multiple ORFs are used, they can be separated by ribosomal binding sides. In some embodiments, the coding sequence of the protein of interest is fused to the coding sequence of streptavidin. In some embodiments, the DNA template is biotinylated through a linker. The linker can be cleavable.

During in vitro transcription/translation, the protein can be expressed in a micelle. In one non-limiting illustrative embodiment, the DNA template contains the coding sequence of a candidate neoantigen, and the candidate neoantigen is expressed in a micelle. In embodiments, a candidate neoantigen forms and is associated with the DNA encoding the candidate neoantigen through a linkage, such as a streptavidin-biotin link. The DNA displayed protein of interest, such as a candidate neoantigen, can be recovered from the emulsion and screened by affinity selection. The DNA template of the selected DNA-displayed protein of interest can be cleaved from the complex by methods such as UV irradiation and then PCR amplified, cloned, and/or sequenced.

In some embodiments, the methods provided herein include generating a library of nucleic acid members, each member encoding a prospective protein of interest (i.e., a candidate neoantigen) having a primary amino acid different from the other proteins encoded by the other nucleic acid members. In some embodiments, the library comprises DNA members, and the method includes transcribing the library into RNA and translating the RNA in a cell free protein synthesis system to generate a bicistronic DNA display system in a water-in-oil emulsion. In some embodiments, the bicistronic DNA display system contains a population of proteins of interest that are selected for specific binding to a binding pair ligand. In one embodiment, the bicistronic DNA display system contains a population of candidate neoantigens, wherein each candidate neoantigen is associated with a DNA molecule encoding the candidate neoantigen.

### P2A DNA Display

In P2A DNA display, which utilizes the cis-activity of the endonuclease P2A, a fusion protein of P2A and a protein of interest binds to the same DNA molecule from which it is expressed (see, e.g., Reiersen et al., Nucleic Acids Research, 33(1): e10). The DNA template can contain the coding sequence encoding the protein of interest genetically fused to the coding sequence of P2A, a promoter, and an origin of replication. The coding sequence of P2A can be obtained and a genetic fusion with the protein of interest can be constructed by standard methods known to those in the art.

In some embodiments, P2A DNA display is used to select a protein of interest. The protein of interest can be selected by generating a library of fusion proteins, where each member of the library comprises a fusion protein between P2A and a protein of interest, and selecting proteins of interest based on a desired property, for example, specific binding to a binding pair ligand. The library can be constructed from a library of nucleic acid members, where each member comprises a DNA template encoding a different protein of interest (i.e., each protein of interest has a primary amino acid sequence that is different from other proteins encoded by other members of the nucleic acid library) that is genetically fused to the coding sequence of P2A.

In some embodiments, the P2A DNA-displayed library can be screened by affinity selection strategies, e.g., in solution or on a solid support. The selected protein of interest can be purified by, e.g., affinity chromatography, and the complexed DNA can be PCR amplified, cloned and/or sequenced.

### CIS Display

Similar to P2A DNA display, CIS display involves a DNA-based approach to directly link in vitro transcribed/translated proteins to the DNA molecules that encode them (see, e.g., Odegrip et al., Proc. Natl. Acad. Sci. USA, 101(9):2806-2810). This method uses RepA, a DNA replication initiator protein, to non-covalently bind to the DNA molecule from which it is expressed if the DNA molecule has a CIS element. The DNA molecule can be created to encode proteins of interest (e.g., candidate neoantigens) in addition to RepA.

In some embodiments, CIS display is used to select a protein of interest. The protein of interest can be selected by generating a DNA library of fusion proteins, where each member comprises a fusion protein between RepA and a protein of interest, and selecting proteins of interest based on a desired property, for example, specific binding to a binding pair ligand. The library can be constructed from a DNA library, where each member of the library comprises a DNA template encoding a different protein of interest (i.e., each protein of interest has a primary amino acid sequence that is different from other proteins encoded by other members of the nucleic acid library) that is genetically fused to the coding sequence of RepA.

In some embodiments, members of the DNA library contain a coding sequence of the protein of interest, a coding sequence of RepA, a CIS element, an origin of replication and a promoter, wherein the coding sequence of the protein of interest is genetically fused to the coding sequence of RepA. The CIS element can be genetically linked to the RepA coding sequence.

In some embodiments, the library contains a fusion protein between RepA and a protein of interest, and a DNA molecule encoding the fusion protein.

The RepA DNA-displayed library can be screened by affinity selection strategies, e.g., in solution or on a solid support. The selected protein of interest can be purified by, e.g., affinity chromatography, and the complexed DNA can be PCR amplified, cloned and/or sequenced.

### Cell-Free Protein Synthesis (CFPS)

In order to express the biologically active proteins of interest described herein (i.e., neoantigens), a cell free protein synthesis system can be used. Cell extracts have been developed that support the synthesis of proteins in vitro from purified mRNA transcripts or from mRNA transcribed from DNA during the in vitro synthesis reaction.

CFPS of polypeptides in a reaction mix comprises bacterial extracts and/or defined reagents. The reaction mix comprises at least ATP or an energy source; a template for production of the macromolecule, e.g., DNA, mRNA, etc.; amino acids, and such co-factors, enzymes and other reagents that are necessary for polypeptide synthesis, e.g., ribosomes, tRNA, polymerases, transcriptional factors, aminoacyl synthetases, elongation factors, initiation factors, etc. In one embodiment of the invention, the energy source is a homeostatic energy source. Also included may be enzyme(s) that catalyze the regeneration of ATP from high-energy phosphate bonds, e.g., acetate kinase, creatine kinase, etc. Such enzymes may be present in the extracts used for translation, or may be added to the reaction mix. Such synthetic reaction systems are well-known in the art, and have been described in the literature.

The templates for cell-free protein synthesis can be either mRNA or DNA. The template can comprise sequences for any particular gene of interest, and may encode a full-length polypeptide or a fragment of any length thereof. Nucleic acids that serve as protein synthesis templates are optionally derived from a natural source or they can be synthetic or recombinant.

For example, DNAs can be recombinant DNAs, e.g., plasmids, viruses or the like.

The term “reaction mix” as used herein, refers to a reaction mixture capable of catalyzing the synthesis of polypeptides from a nucleic acid template. The reaction mixture comprises extracts from bacterial cells, e.g., E. coli S30 extracts. S30 extracts are well known in the art, and are described in, e.g., Lesley, S. A., et al. (1991), J. Biol. Chem. 266, 2632-8. The synthesis can be performed under either aerobic or anaerobic conditions.

In some embodiments, the bacterial extract is dried. The dried bacterial extract can be reconstituted in milli-Q water (e.g., reverse osmosis water) at 110% of the original solids as determined by measuring the percent solids of the starting material. In one embodiment, an accurately weighed aliquot of dried extract, representing 110% of the original solids of 10 mL of extract, is added to 10 mL of Milli-Q water in a glass beaker with a stir bar on a magnetic stirrer. The resulting mixture is stirred until the powder is dissolved. Once dissolved, the material is transferred to a 15 mL Falcon tube and stored at −80 C unless used immediately.

The volume percent of extract in the reaction mix will vary, where the extract is usually at least about 10% of the total volume; more usually at least about 20%; and in some instances may provide for additional benefit when provided at least about 50%; or at least about 60%; and usually not more than about 75% of the total volume.

The general system includes a nucleic acid template that encodes a protein of interest. The nucleic acid template is an RNA molecule (e.g., mRNA) or a nucleic acid that encodes an mRNA (e.g., RNA, DNA) and be in any form (e.g., linear, circular, supercoiled, single stranded, double stranded, etc.). Nucleic acid templates guide production of the desired protein.

To maintain the template, cells that are used to produce the extract can be selected for reduction, substantial reduction or elimination of activities of detrimental enzymes or for enzymes with modified activity. Bacterial cells with modified nuclease or phosphatase activity (e.g., with at least one mutated phosphatase or nuclease gene or combinations thereof) can be used for synthesis of cell extracts to increase synthesis efficiency. For example, an E. coli strain used to make an S30 extract for CFPS can be RNase E or RNase A deficient (for example, by mutation).

In a generic CFPS reaction, a gene encoding a protein of interest is expressed in a transcription buffer, resulting in mRNA that is translated into the protein of interest in a CFPS extract and a translation buffer. The transcription buffer, cell-free extract and translation buffer can be added separately, or two or more of these solutions can be combined before their addition, or added contemporaneously.

To synthesize a protein of interest in vitro, a CFPS extract at some point comprises a mRNA molecule that encodes the protein of interest. In some CFPS systems, mRNA is added exogenously after being purified from natural sources or prepared synthetically in vitro from cloned DNA using RNA polymerases such as RNA polymerase II, SP6 RNA polymerase, T3 RNA polymerase, T7 RNA polymerase, RNA polymerase III and/or phage derived RNA polymerases. In other systems, the mRNA is produced in vitro from a template DNA; both transcription and translation occur in this type of CFPS reaction. In some embodiments, the transcription and translation systems are coupled or comprise complementary transcription and translation systems, which carry out the synthesis of both RNA and protein in the same reaction. In such in vitro transcription and translation systems, the CFPS extracts contain all the components (exogenous or endogenous) necessary both for transcription (to produce mRNA) and for translation (to synthesize protein) in a single system.

A cell free protein synthesis reaction mixture comprises the following components: a template nucleic acid, such as DNA, that comprises a gene of interest operably linked to at least one promoter and, optionally, one or more other regulatory sequences (e.g., a cloning or expression vector containing the gene of interest) or a PCR fragment; an RNA polymerase that recognizes the promoter(s) to which the gene of interest is operably linked and, optionally, one or more transcription factors directed to an optional regulatory sequence to which the template nucleic acid is operably linked; ribonucleotide triphosphates (rNTPs); optionally, other transcription factors and co-factors therefor; ribosomes; transfer RNA (tRNA); other or optional translation factors (e.g., translation initiation, elongation and termination factors) and co-factors therefore; one or more energy sources, (e.g., ATP, GTP); optionally, one or more energy regenerating components (e.g., PEP/pyruvate kinase, AP/acetate kinase or creatine phosphate/creatine kinase); optionally factors that enhance yield and/or efficiency (e.g., nucleases, nuclease inhibitors, protein stabilizers, chaperones) and co-factors therefore; and;

optionally, solubilizing agents. The reaction mix further comprises amino acids and other materials specifically required for protein synthesis, including salts (e.g., potassium, magnesium, ammonium, and manganese salts of acetic acid, glutamic acid, or sulfuric acids), polymeric compounds (e.g., polyethylene glycol, dextran, diethyl aminoethyl dextran, quaternary aminoethyl and aminoethyl dextran, etc.), cyclic AMP, inhibitors of protein or nucleic acid degrading enzymes, inhibitors or regulators of protein synthesis, oxidation/reduction adjuster (e.g., DTT, ascorbic acid, glutathione, and/or their oxides), non-denaturing surfactants (e.g., Triton X-100), buffer components, spermine, spermidine, putrescine, etc. Components of CFPS reactions are discussed in more detail in U.S. Pat. Nos. 7,338,789 and 7,351,563, and U.S. App. Pub. No. 2010/0184135, the disclosures of which are incorporated by reference in its entirety for all purposes.

Depending on the specific enzymes present in the extract, for example, one or more of the many known nuclease, polymerase or phosphatase inhibitors can be selected and advantageously used to improve synthesis efficiency.

Protein and nucleic acid synthesis typically requires an energy source. Energy is required for initiation of transcription to produce mRNA (e.g., when a DNA template is used and for initiation of translation high energy phosphate for example in the form of GTP is used). Each subsequent step of one codon by the ribosome (three nucleotides; one amino acid) requires hydrolysis of an additional GTP to GDP. ATP is also typically required. For an amino acid to be polymerized during protein synthesis, it must first be activated. Significant quantities of energy from high energy phosphate bonds are thus required for protein and/or nucleic acid synthesis to proceed.

An energy source is a chemical substrate that can be enzymatically processed to provide energy to achieve desired chemical reactions. Energy sources that allow release of energy for synthesis by cleavage of high-energy phosphate bonds such as those found in nucleoside triphosphates, e.g., ATP, are commonly used. Any source convertible to high energy phosphate bonds is especially suitable. ATP, GTP, and other triphosphates can normally be considered as equivalent energy sources for supporting protein synthesis.

To provide energy for the synthesis reaction, the system can include added energy sources such as glucose, pyruvate, phosphoenolpyruvate (PEP), carbamoyl phosphate, acetyl phosphate, creatine phosphate, phosphopyruvate, glyceraldehyde-3-phosphate, 3-Phosphoglycerate and glucose-6-phosphate that can generate or regenerate high-energy triphosphate compounds such as ATP, GTP, other NTPs, etc.

When sufficient energy is not initially present in the synthesis system, an additional source of energy is preferably supplemented. Energy sources can also be added or supplemented during the in vitro synthesis reaction.

In some embodiments, the cell-free protein synthesis reaction is performed using the PANOx-SP system comprising NTPs, E. coli tRNA, amino acids, Mg²⁺ acetate, Mg²⁺ glutamate, K⁺ acetate, K⁺ glutamate, folinic acid, Tris pH 8.2, DTT, pyruvate kinase, T7 RNA polymerase, disulfide isomerase, phosphoenol pyruvate (PEP), NAD, CoA, Na⁺ oxalate, putrescine, spermidine, and S30 extract.

In some instances, the cell-free synthesis reaction does not require the addition of commonly secondary energy sources, yet uses co-activation of oxidative phosphorylation and protein synthesis. In some instances, CFPS is performed in a reaction such as the Cytomim (cytoplasm mimic) system. The Cytomim system is defined as a reaction condition performed in the absence of polyethylene glycol with optimized magnesium concentration. This system does not accumulate phosphate, which is known to inhibit protein synthesis.

The presence of an active oxidative phosphorylation pathway can be tested using inhibitors that specifically inhibit the steps in the pathway, such as electron transport chain inhibitors. Examples of inhibitors of the oxidative phosphorylation pathway include toxins such as cyanide, carbon monoxide, azide, carbonyl cyanide m-chlorophenyl hydrazone (CCCP), and 2,4-dinitrophenol, antibiotics such as oligomycin, pesticides such as rotenone, and competitive inhibitors of succinate dehydrogenase such as malonate and oxaloacetate.

In some embodiments, the cell-free protein synthesis reaction is performed using the Cytomim system comprising NTPs, E. coli tRNA, amino acids, Mg²⁺ acetate, Mg²⁺ glutamate, K⁺ acetate, K⁺ glutamate, folinic acid, Tris pH 8.2, DTT, pyruvate kinase, T7 RNA polymerase, disulfide isomerase, sodium pyruvate, NAD, CoA, Na⁺ oxalate, putrescine, spermidine, and S30 extract. In some embodiments, the energy substrate for the Cytomim system is pyruvate, glutamic acid, and/or glucose. In some embodiments of the system, the nucleoside triphosphates (NTPs) are replaced with nucleoside monophosphates (NMPs).

The cell extract can be treated with iodoacetamide in order to inactivate enzymes that can reduce disulfide bonds and impair proper protein folding. In some embodiments, the cell extract comprises an exogenous protein chaperone. The protein chaperone can be expressed by the bacterial strain used to make the cell free extract, or the protein chaperone can be added to the cell extract. Non-limiting examples of exogenous protein chaperones include disulfide bond isomerase (PDI), such as, but not limited to E. coli DsbC, and peptidyl prolyl cis-trans isomerase (PPlase), such as but not limited to FkpA. In some embodiments, the extract comprises both a PDI and a PPIase, e.g., both DsbC and FkpA. Glutathione disulfide (GSSG) and/or glutathione (GSH) can also be added to the extract at a ratio that promotes proper protein folding and prevents the formation of aberrant protein disulfides.

In some embodiments, the CFPS reaction includes inverted membrane vesicles to perform oxidative phosphorylation. These vesicles can be formed during the high pressure homogenization step of the preparation of cell extract process, as described herein, and remain in the extract used in the reaction mix.

Methods of preparing a cell extract are described in, e.g., Zawada, J. “Preparation and Testing of E. coli S30 In Vitro Transcription Translation Extracts”, Douthwaite, J. A. and Jackson, R. H. (eds.), Ribosomal display and Related Technologies: Methods and Protocols, Methods in Molecular Biology, vol. 805, pp. 31-41 (Humana Press, 2012); Jewett et al., Molecular Systems Biology: 4, 1-10 (2008); Shin J. and Norieaux V., J. Biol. Eng., 4:8 (2010). Briefly, a bacterial culture is grown and harvested; suspended in an appropriate buffer (e.g., S30 buffer), and homogenized to lyse the cells.

The cell-free extract can be thawed to room temperature before use in the CFPS reaction. The extract can be incubated with 50 μM iodoacetamide for 30 minutes when synthesizing protein with disulfide bonds. In some embodiments, the CFPS reaction includes about 30% (v/v) iodoacetamide-treated extract with about 8 mM magnesium glutamate, about 10 mM ammonium glutamate, about 130 mM potassium glutamate, about 35 mM sodium pyruvate, about 1.2 mM AMP, about 0.86 mM each of GMP, UMP, and CMP, about 2 mM amino acids (about 1 mM for tyrosine), about 4 mM sodium oxalate, about 0.5 mM putrescine, about 1.5 mM spermidine, about 16.7 mM potassium phosphate, about 100 mM T7 RNA polymerase, about 2-10 μg/mL plasmid DNA template, about 1-10 μM E. coli DsbC, and a total concentration of about 2 mM oxidized (GSSG) glutathione. Optionally, the cell free extract can include 1 mM of reduced (GSH).

### Generating a Lysate

The methods and systems described herein can use a cell lysate for in vitro translation of a target protein of interest. For convenience, the organism used as a source for the lysate may be referred to as the source organism or host cell. Host cells may be bacteria, yeast, mammalian or plant cells, or any other type of cell capable of protein synthesis. A lysate comprises components that are capable of translating messenger ribonucleic acid (mRNA) encoding a desired protein, and optionally comprises components that are capable of transcribing DNA encoding a desired protein. Such components include, for example, DNA-directed RNA polymerase (RNA polymerase), any transcription activators that are required for initiation of transcription of DNA encoding the desired protein, transfer ribonucleic acids (tRNAs), aminoacyl-tRNA synthetases, 70S ribosomes, N¹⁰-formyltetrahydrofolate, formylmethionine-tRNAf^{Met} synthetase, peptidyl transferase, initiation factors such as IF-1, IF-2, and IF-3, elongation factors such as EF-Tu, EF-Ts, and EF-G, release factors such as RF-1, RF-2, and RF-3, and the like.

An embodiment uses a bacterial cell from which a lysate is derived. A bacterial lysate derived from any strain of bacteria can be used in the methods of the invention. The bacterial lysate can be obtained as follows. The bacteria of choice are grown to log phase in any of a number of growth media and under growth conditions that are well known in the art and easily optimized by a practitioner for growth of the particular bacteria. For example, a natural environment for synthesis utilizes cell lysates derived from bacterial cells grown in medium containing glucose and phosphate, where the glucose is present at a concentration of at least about 0.25% (weight/volume), more usually at least about 1%; and usually not more than about 4%, more usually not more than about 2%. An example of such media is 2YTPG medium, however one of skill in the art will appreciate that many culture media can be adapted for this purpose, as there are many published media suitable for the growth of bacteria such as E. coli, using both defined and undefined sources of nutrients. Cells that have been harvested overnight can be lysed by suspending the cell pellet in a suitable cell suspension buffer, and disrupting the suspended cells by sonication, breaking the suspended cells in a French press, continuous flow high pressure homogenization, or any other method known in the art useful for efficient cell lysis. The cell lysate is then centrifuged or filtered to remove large DNA fragments and cell debris.

The bacterial strain used to make the cell lysate generally has reduced nuclease and/or phosphatase activity to increase cell free synthesis efficiency. For example, the bacterial strain used to make the cell free extract can have mutations in the genes encoding the nucleases RNase E and RNase A. The strain may also have mutations to stabilize components of the cell synthesis reaction such as deletions in genes such as tnaA, speA, sdaA or gshA, which prevent degradation of the amino acids tryptophan, arginine, serine and cysteine, respectively, in a cell-free synthesis reaction. Additionally, the strain may have mutations to stabilize the protein products of cell-free synthesis such as knockouts in the proteases ompT or lonP.

### DNA Expression Cassette Without Stop Codon

In some embodiments, the nucleic acid template used in the ribosomal display reaction system comprises a DNA expression cassette that is capable of expressing an RNA encoding the protein of interest, where the RNA lacks an operable stop codon. To remove the stop codon from the protein coding region in the expression cassette, a PCR primer lacking the stop codon can be used to amplify the coding region for the protein of interest, such that the entire coding region from the translation start to the sequence encoding the C-terminal amino acid is amplified.

MHC Binding Assay with Neoantigen Library

The major histocompatibility complex (MHC) is a collection of genes encoding glycoproteins called MHC proteins. The primary function of an MHC protein in vivo is to present antigen in a form capable of being recognized by a TCR. An MHC protein is bound to an antigen in the form of an antigenic peptide to form an MHC-peptide complex.

MHC proteins, also known as human leukocyte antigen (HLA) in humans and H-2 region in mice, are classified in two categories: class I and class II MHC proteins. These proteins are comprised of a cluster of highly polymorphic genes. Specifically, human HLA-A, HLA-B, and HLA-C are known as class I MHC molecules, whereas human HLA-DP, HLA-DQ and HLA-DR are known as class II MHC molecules. The HLA loci include HLA-DP, HLA-DN, HLA-DM, HLA-DO, HLA-DQ, HLA-DR, HLA-A, HLA-B and HLA-C. Each of these loci contains different alleles in the human population. The different subtypes encoded by these allelic variants are intended to be within the scope of the invention.

An MHC class II protein is a heterodimeric integral membrane protein comprising one α and one β chain in noncovalent association. The α chain has two extracellular domains (α₁ and α₂), and a transmembrane (TM) and a cytoplasmic (CYT) domain. The β chain contains two extracellular domains (β₁ and β₂), and a TM and CYT domain. An MHC class I protein is an integral membrane protein comprising a glycoprotein heavy chain having three extracellular domains (i.e., α₁, α₂ and α₃), and a TM and CYT domain. The heavy chain is noncovalently associated with a soluble subunit called β₂-microglobulin (β₂ₘ).

In some embodiments, the binding assay (e.g., the MHC-PepSeq assay) of the present invention employs the use of a spaceholder molecule linked to the MHC class II component, e.g., the β chain, by a processable linker. A spaceholder molecule of the present invention may be any peptide that is capable of binding to a peptide binding groove of an MHC protein in such a manner that binding of any other peptide in the peptide binding groove is hindered. Preferably, the spaceholder molecule binds within the peptide binding groove with intermediate affinity, and more preferably with low affinity, at approximately neutral pH. In one embodiment, the length of a spaceholder molecule extends from about 5 to about 40 amino acid residues, more preferably from about 6 to about 30 amino acid residues, 8 to about 20 amino acid residues and even more preferably from about 12 to 15 amino acids residues. In a further embodiment, a spaceholder molecule is about 13 amino acids residues. Examples of suitable spaceholder molecules include, without limitation, (in single letter amino acid code) PVSKMRMATPLLMQA (SEQ ID NO: 73), also known as CLIP; AAMAAAAAAAMAA (SEQ ID NO: 74); AAMAAAAAAAAAA (SEQ ID NO: 75); AAFAAAAAAAAAA (SEQ ID NO: 76); ASMSAASAASMAA (SEQ ID NO: 77), and functional equivalents thereof. In one embodiment, the spaceholder molecule will have the consensus sequence AAXAAAAAAAXAA (SEQ ID NO: 78), wherein X is any amino acid. The ability of maintaining a spaceholder molecule within the binding groove of the MHC class II molecule prevents “empty” molecules from forming. The formation of “empty” MHC class II molecules has been a major limiting factor due to the tendency of these “empty” molecules to aggregate, thus making isolation of functional MHC class II components difficult.

The spaceholder molecule of the present invention may be covalently linked to the MHC molecule by a linker having an amino acid sequence that contains a target site for an enzyme capable of cleaving proteins. Such linkers are referred to herein as “processable linkers”. Examples of processable linkers of the present invention include linkers containing target sites for enzymes such as collagenases, metalloproteases, serine proteases, cysteine proteases, kallikriens, thrombin, and plasminogen activators. A preferred processable linker of the present invention includes a linker having a thrombin cleavage site.

Suitable linkers useful in the present invention can also be designed using various methods. For example, X-ray crystallographic data of an MHC protein can be used to design a linker of suitable length and charge such that the linker does not interfere with the binding of the spaceholder molecule to the peptide binding groove of the MHC class II component. Such methods are included in the present invention.

The length of a linker of the present invention is preferably sufficiently short (i.e., small enough in size) such that the linker does not substantially inhibit binding between the spaceholder molecule and the MHC class II component. The length of a linker of the present invention may range from about 1 amino acid residue to about 40 amino acid residues, more preferably from about 5 amino acid residues to about 30 amino acid residues, and even more preferably from about 8 amino acid residues to about 20 amino acid residues.

The cleavage of the linker facilitates the release of the spaceholder molecule thereby freeing the peptide binding groove. The MHC class II component may then be incubated with the library of candidate neoantigens (e.g., a PepSeq library of peptides designed from exome analysis of tumor and wild-type cells) to facilitate the binding of the antigen peptide molecule to the MHC class II component. After sufficient time to allow binding, which can be readily determined by one skilled in the art, the MHC class II component which has bound to the antigen peptide molecule is recovered.

In certain embodiments, the step of cleaving the linker and incubating with the antigen peptide molecule is repeated using different antigen peptide molecules. The advantage of repeating these steps is to allow for the formation of a number of MHC class II components that recognize several antigenic epitopes. Furthermore, these steps can be carried out using MHC class II molecules constructed with varying allelic forms of the MHC class II genes. This feature of the assay therefore allows for the generation of several MHC class II components which are specific for a number of different MHC allelic genotypes.

Alternatively, the binding assay (e.g., the MHC-PepSeq assay) of the present invention employs a phage display system (see, for example, Hammer J et al., Promiscuous and allele-specific anchors in HLA-DR-binding peptides, Cell (1993) 74(1):197-203). MHC II components are expressed with phage display. The phages expressing the MHC II components are then incubated with the library of candidate neoantigens (e.g., a PepSeq library of peptides designed from exome analysis of tumor and wild-type cells) to facilitate the binding of the antigen peptide molecule to the MHC class II component. After sufficient time to allow binding, which can be readily determined by one skilled in the art, the MHC class II component which has bound to the antigen peptide molecule is recovered.

In certain aspects, an in silico analysis is used to determine specific binding between neoantigens and MHC class I proteins or fragments thereof. The in silico analysis comprises applying a computational algorithm to predict relative binding to MHC I proteins based on the peptide sequences of the neoantigens. Tools such as netMHCpan are used for prediction of binding of peptides/neoantigens to the MHC proteins (see Jurtz et al, NetMHCpan-4.0: Improved Peptide-MHC Class I Interaction Predictions Integrating Eluted Ligand and Peptide Binding Affinity Data, J Immunol (2017) 199(9):3360-3368). The input to this analysis is the peptide sequences and the MHC alleles of interest, and the output is a predicted binding affinity.

Incubation of TILs with Neoantigens

In certain aspects, neoantigens demonstrating binding with MHC class I molecules or MHC class II molecules in an MHC binding assay or in silico are pooled to form a peptide mixture and this peptide mixture is incubated with TILS isolated from the patient's tumor. After incubation with the peptide mixture, a peptide-specific enriched TIL population is generated (see FIG. 2).

In some aspects, the TILS are incubated with the peptide mixture for about 6 hours, 12 hours, 18 hours, 24 hours, 48 hours, or 72 hours. In other aspects, the TILS are incubated with the peptide mixture for about 1 day, 2 days, 3 days, 4 days, 5 days, 6 days, 7 days, 8 days, 9 days, 10 days, or more. In yet other aspects, the TILS are incubated with the peptide mixture for between 1 and 10 days (e.g., between 1 and 10 days, between 1 and 9 days, between 1 and 8 days, between 1 and 7 days, between 1 and 6 days, between 1 and 5 days, etc.).

In one aspect, about 2.5 x 10⁶ TILs are cultured in RPMI 1640 supplemented with HEPES, L-Glutamine, human serum, sodium pyruvate, non-essential amino acids, and 2-mercaptoethanol in a 6-well tissue treated plate in the presence of about 2.5 μg/mL peptide mixture for 8 days followed by REP (rapid expansion).

### Expansion of Neoantigen-Specific TILs

In certain aspects, neoantigen-specific TIL production is a 2-step process. The pre-REP (Rapid Expansion) stage is where the TILs are grown in standard lab media such as RPMI and treat the TILs with reagents such as irradiated feeder cells, and anti-CD3 antibodies to achieve the desired effect. The REP stage is where the TILs are expanded in a large enough culture amount for treating the patients.

In some embodiments, neoantigen-specific TIL production is a rapid expansion stage where the TILs are grown in standard lab media such as RPMI 1640 supplemented with HEPES, L-Glutamine, human serum, sodium pyruvate, non-essential amino acids, 2-mercaptoethanol, IL-2 (or other cytokines), and anti-CD3 antibodies, and cultured with irradiated feeder cells to achieve the desired effect.

Expansion of tumor-infiltrating lymphocytes, such as T cells can be accomplished by any of the methods known in the art. For example, T cells can be rapidly expanded using non-specific T-cell receptor stimulation in the presence of feeder lymphocytes and interleukin-2 (IL-2), IL-7, IL-15, IL-21, or combinations thereof. The non-specific T-cell receptor stimulus can include around 30 ng/ml of OKT3, a mouse monoclonal anti-CD3 antibody (available from Ortho-McNeil®, Raritan, N.J. or Miltenyi Biotec, Bergisch Gladbach, Germany). Alternatively, T cells can be rapidly expanded by stimulation of peripheral blood mononuclear cells (PBMC) in vitro with one or more antigens (including antigenic portions thereof, such as epitope(s), or a cell of the cancer, which can be optionally expressed from a vector, such as an human leukocyte antigen A2 (HLA-A2) binding peptide, e.g., approximately 0.3 μM MART-1:26-35 (27 L) or gp100:209-217 (210M)), in the presence of a T-cell growth factor, such as around 200-400 Ill/ml, such as 300 IU/ml IL-2 or IL-15, with IL-2 being preferred. The in vitro-induced T-cells are rapidly expanded by re-stimulation with the same antigen(s) of the cancer pulsed onto HLA-A2-expressing antigen-presenting cells. Alternatively, the T-cells can be re-stimulated with irradiated, autologous lymphocytes or with irradiated HLA-A2+ allogeneic lymphocytes and IL-2, for example.

Alternatively or in addition, TIL can be rapidly expanded using non-specific T-cell receptor stimulation in the presence of feeder cells (e.g., irradiated allogeneic feeder cells, irradiated autologous feeder cells, and/or artificial antigen presenting cells (e.g., K562 leukemia cells transduced with nucleic acids encoding CD3 and/or CD8)) and either interleukin-2 (IL-2) or interleukin-15 (IL-15), with IL-2 being preferred. In an embodiment of the method, expanding the number of TIL uses about 1×10⁹ to about 4×10⁹ allogeneic feeder cells and/or autologous feeder cells, preferably about 2×10⁹ to about 3×10⁹ allogeneic feeder cells and/or autologous feeder cells. The non-specific T-cell receptor stimulus can include, for example, about 30 ng/ml of OKT3, a mouse monoclonal anti-CD3 antibody (available from Ortho-Mcneil, Raritan, N.J. or Miltenyi Biotech, Auburn, Calif). Alternatively, TIL can be rapidly expanded by, for example, stimulation of the TIL in vitro with an antigen (one or more, including antigenic portions thereof, such as epitope(s), or a cell) of the cancer, which can be optionally expressed from a vector, such as a human leukocyte antigen A2 (HLA-A2) binding peptide, e.g., 0.3 μM MART-1:26-35 (27L) or gp100:209-217 (210M), in the presence of a T-cell growth factor, such as 300 IU/ml IL-2 or IL-15, with IL-2 being preferred. Other suitable antigens may include, e.g., NY-ESO-1, TRP-1, TRP-2, tyrosinase cancer antigen, MAGE-A3, SSX-2, and VEGFR2, or antigenic portions thereof. Additionally, the neoantigens identified with the methods described herein may be used. The in vitro-induced TILs are rapidly expanded by re-stimulation with the same antigen(s) of the cancer pulsed onto HLA-A2-expressing antigen-presenting cells. Alternatively, the TIL can be re-stimulated with, for example, irradiated, autologous lymphocytes or with irradiated HLA-A2+ allogeneic lymphocytes and IL-2, for example.

Specific tumor reactivity of the expanded TILs can be tested by any method known in the art, e.g., by measuring cytokine release (e.g., interferon-gamma) following co-culture with tumor cells. In some embodiments, a T-cell growth factor that promotes the growth and activation of the autologous T cells is administered to the mammal either concomitantly with the autologous T cells or subsequently to the autologous T cells. The T-cell growth factor can be any suitable growth factor that promotes the growth and activation of the autologous T-cells. Examples of suitable T-cell growth factors include interleukin (IL)-2, IL-7, IL-15, IL-12, and IL-21, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

### Genome Editing of TILs

Introduction of the genetic sequences for the TCR recognizing the neoantigen into the universal donor T cells is accomplished with (a) meganucleases, (b) zinc finger nucleases (ZFNs), (c) transcription activator-like effector-based nucleases (TALENs), (d) mega-TALENs or (e) the CRISPR-Cas system. Techniques using each of these engineered nucleases are well known in the art.

In some embodiments, the TILs are modified to suppress expression of one or more genes. In some embodiments, the TILs are further modified via genome editing. Various methods and compositions for targeted cleavage of genomic DNA have been described. Such targeted cleavage events can be used, for example, to induce targeted mutagenesis, induce targeted deletions of cellular DNA sequences; U.S. Pat. Nos. 8,110,379; 8,409,861 ; 8,586,526; U.S. Patent Publications 20030232410; 20050208489; 20050026157; 20050064474; 20060063231; 201000218264; 20120017290; 20110265198; 20130137104; 20130122591; 20130177983 and 20130177960, the disclosures of which are incorporated by reference in their entireties.

These methods often involve the use of engineered cleavage systems to induce a double strand break (DSB) or a nick in a target DNA sequence such that repair of the break by an error born process such as non-homologous end joining (NHEJ) or repair using a repair template (homology directed repair or HDR) can result in the knockout of a gene or the insertion of a sequence of interest (targeted integration). Cleavage can occur through the use of specific nucleases such as engineered zinc finger nucleases (ZFN), transcription-activator like effector nucleases (TALENs), or using the CRISPR/Cas system with an engineered crRNA/tracr RNA (‘single guide RNA’) to guide specific cleavage.

In some embodiments, the TILs are modified to disrupt or reduce expression of an endogenous T-cell receptor gene (see, e.g. WO 2014/153470). In some embodiments, the engineered immune cells are modified to result in disruption or inhibition of PD1, PDL-1 or CTLA-4 (see, e.g. U.S. Patent Publication 20140120622), or other immunosuppressive factors known in the art (Wu et al. (2015) Oncoimmunology 4(7): e1016700, Mahoney et al. (2015) Nature Reviews Drug Discovery 14, 561-584).

Universal donor cells for parallel gene transfer of patient-specific neoantigen reactivity may be derived from any source of T cells. In one aspect, the universal donor T cells are generated from a cord blood T cells. The use of cord blood T cells is advantageous to the invention because they have a naive phenotype, an immense proliferative potential and potent in vivo activity in transplant recipients. Thus, production of universal donor cells may begin with obtaining a sample of cord blood. The sample of cord blood may be any type of sample. For instance, the sample of cord blood may be fresh cord blood or frozen cord blood. The sample of cord blood may have been derived from one individual. The sample of cord blood may have been derived from multiple individuals, for example, a pooled cord blood sample.

Cord blood T cells may be obtained from the cord blood sample by separating cells that express CD62L from the sample. Any appropriate method may be used to separate cells that express CD62L from the sample. For instance, the cells that express CD62L may be separated from the sample based on their ability to bind an anti-CD62L antibody.

Fluorescence activated cell sorting (FACS) or magnetic activated cell sorting (MACS) may be used to separate the cells that express CD62L from the sample. In MACS, magnetic beads are conjugated to the anti-CD62L antibody. Binding of the CD62L-expressing cells to the anti-CD62L antibody therefore tags the cells with magnetic beads. Magnetism can therefore be used to separate the tagged cells from the sample.

The separation step may be manually performed. Alternatively, the separation step may be performed in a system designed for the automated separation of cells. In one aspect, the system is configured for automated production of cord T cells. The system may be a CliniMacs system or a Miltenyi Prodigy system. Other automated cell separation systems are known in the art.

### TIL Administration

The administration of the TILs according to the methods provided herein can be carried out in any suitable manner for administering cells to a subject, including but not limited to injection, transfusion, implantation, or transplantation. In some embodiments, the TILs are administered to a patient subcutaneously, intradermally, intratumorally, intranodally, intramedullary, intramuscularly, intrathecally, by intravenous or intralymphatic injection, or intraperitoneally. In some embodiments, the TILs are administered into a cavity formed by the resection of tumor tissue (for example, intracavity delivery) or directly into a tumor prior to resection (for example, intratumoral delivery). In one embodiment, the TILs are administered by intravenous injection.

In an embodiment, the invention includes a method of treating a cancer with a population of TILs, wherein a patient is pre-treated with non-myeloablative chemotherapy prior to an infusion of TILs according to the invention. In some embodiments, the population of TILs may be provided wherein a patient is pre-treated with nonmyeloablative chemotherapy prior to an infusion of TILs according to the present invention.

In an embodiment, the non-myeloablative chemotherapy is cyclophosphamide 60 mg/kg/d for 2 days (days 27 and 26 prior to TIL infusion) and fludarabine 25 mg/m2/d for 5 days (days 27 to 23 prior to TIL infusion). In an embodiment, after non-myeloablative chemotherapy and TIL infusion (at day 0) according to the invention, the patient receives an intravenous infusion of IL-2 intravenously at 720,000 IU/kg every 8 hours to physiologic tolerance.

Experimental findings indicate that lymphodepletion prior to adoptive transfer of tumor-specific T lymphocytes plays a key role in enhancing treatment efficacy by eliminating regulatory T cells and competing elements of the immune system (“cytokine sinks”). Accordingly, some embodiments of the invention utilize a lymphodepletion step (sometimes also referred to as “immunosuppressive conditioning”) on the patient prior to the introduction of the TILs of the invention.

### Methods of Treating Cancer

The cancer treated by the disclosed compositions and methods can be any solid tumor for which TILs can be produced. The cancer can also be metastatic and/or recurrent. Non-limiting examples of cancers include acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the vulva, chronic lymphocytic leukemia, chronic myeloid cancer, cervical cancer, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, peritoneum, omentum, and mesentery cancer, pharynx cancer, prostate cancer, rectal cancer, renal cancer, skin cancer, soft tissue cancer, testicular cancer, thyroid cancer, ureter cancer, urinary bladder cancer, and digestive tract cancer such as, e.g., esophageal cancer, gastric cancer, pancreatic cancer, stomach cancer, small intestine cancer, gastrointestinal carcinoid tumor, cancer of the oral cavity, colorectal cancer, and hepatobiliary cancer.

Triple Negative Breast Cancers (TNBCs), representing about 15% of all breast cancers, are highly aggressive type of tumors that lack estrogen receptor (ER), progesterone receptor (PR), and ERBB2 (HER2) gene amplification. TNBCs do not respond to hormonal therapy such as tamoxifen or aromatase inhibitors or therapies that target HER2 receptors, such as Herceptin (trastuzumab). Because of limited targets that are available for TNBCs, currently there is an intense interest in finding new targets and thus personalized medications that can treat this type of breast cancer. Therefore, in some embodiments, the cancer is a triple negative breast cancer (TNBC).

### Combination Cancer Therapies

The disclosed compositions and methods can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Therapeutic cancer vaccines have been developed to actively drive an antitumor immune response. Unlike the prophylactic vaccines that are used preventatively to treat infectious diseases, therapeutic vaccines are designed to treat established cancer by stimulating an immune response against a specific tumor-associated antigen. In 2010, sipuleucel-T (Provenge; Dendreon Corporation) was approved by the FDA for the treatment of metastatic, castration-resistant prostate cancer based on the results of the IMPACT (Immunotherapy Prostate Adenocarcinoma Treatment) trial in which it improved OS by 4.1 months and reduced the risk of death by 22% versus placebo. The advantage of active immunotherapies is that they have the potential to provide long-lasting anticancer activity by engaging both the innate and adaptive arms of the immune response. While mAbs are typically considered passive immunotherapies, there is increasing evidence that they also induce an adaptive immune response via a “vaccination-like” effect.

Generating an optimal “killer” CD8 T cell response also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.

Numerous anti-cancer drugs are available for combination with the present method and compositions. The following is anon-exhaustive lists of anti-cancer (anti-neoplastic) drugs that can be used in conjunction with or without irradiation: Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

The disclosed compositions and methods can be used in combination with one or more immune checkpoint inhibitors. By immune checkpoint inhibitor it is meant a compound that inhibits a protein in the checkpoint signally pathway. Proteins in the checkpoint signally pathway include for example, CD27, CD28, CD40, CD 122, CD137, OX40, GITR, ICOS, A2AR, B7-H3, B7-H4, BTLA, CTLA-4, IDO, KIR, LAG3, PD-1, PD-L1, PD-L2, TIM-3, TIGIT, Lairl, CD244, HAVCR2, CD200, CD200R1, CD200R2, CD200R4, LILRB4, PILRA, ICOSL, 4-1BB or VISTA. Immune checkpoint inhibitors are known in the art.

For example, the immune checkpoint inhibitor can be a small molecule. Small molecules can be, e.g., nucleic acids, peptides, polypeptides, peptidomimetics, carbohydrates, lipids or other organic or inorganic molecules.

Alternatively, the immune checkpoint inhibitor is an antibody or fragment thereof. For example, the antibody or fragment thereof is specific to a protein in the checkpoint signaling pathway, such as CD27, CD28, CD40, CD 122, CD137, OX40, GITR, ICOS, A2AR, B7-H3, B7-H4, BTLA, CTLA-4, IDO, KIR, LAG3, PD-1, PD-L1, PD-L2, TIM-3, TIGIT, Lairl, CD244, HAVCR2, CD200, CD200R1, CD200R2, CD200R4, LILRB4, PILRA, ICOSL, 4-1BB or VISTA.

Exemplary, anti-immune checkpoint antibodies include for example ipiliumab (anti-CTLA-4), penbrolizumab (anti-PD-L1), nivolumab (anti-PD-L1), atezolizumab (anti-PD-L1), and duralumab (anti-PD-L1).

The present invention is further illustrated by the following examples that should not be construed as limiting. The contents of all references, patents, and published patent applications cited throughout this application, as well as the Figures, are incorporated herein by reference in their entirety for all purposes.

### EXAMPLES

### Example 1

### Overview of Neoantigen-Informed TIL Therapy

TIL (Tumor Infiltrating Lymphocyte) therapy is an autologous cellular immunotherapy in which TILs are isolated from surgically-resected tumor specimens, expanded in vitro, and then reinfused into the patient, following a lymphodepleting conditioning regimen. While TIL therapy has achieved some impressive responses against a range of cancers, its success remains sporadic. On the other hand, there are reasons in principle to think that TIL therapy can be a broadly-applicable therapeutic. First, it is known that the majority of tumors are infiltrated by neoantigen-specific T cells, albeit sometimes at very low frequencies. Second, it has been shown that the expansion and infusion of a highly pure TIL product comprising CD4 or CD8 T cells reactive to a single neoantigen is sufficient to mediate tumor regression.

The Inventors have developed a differentiated NeoTIL approach (FIGS. 1 and 2) in which a novel peptide:MHC screening technology (i.e., “PepSeq”) or related methodology (e.g., ribosomal display) is applied to predict personalized immunogenic neoantigens from genomic analysis, and then these neoantigens are used to enrich TIL preparations for the most potent cells. Personalized NeoTIL therapies can be produced in a relatively short timeline (see FIGS. 3 and 4).

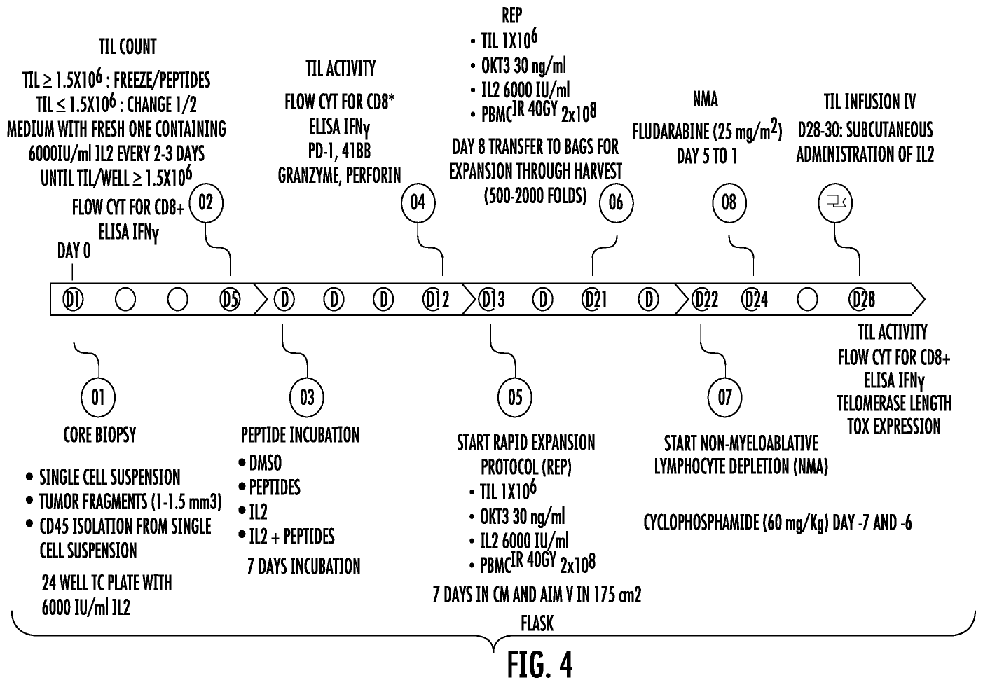

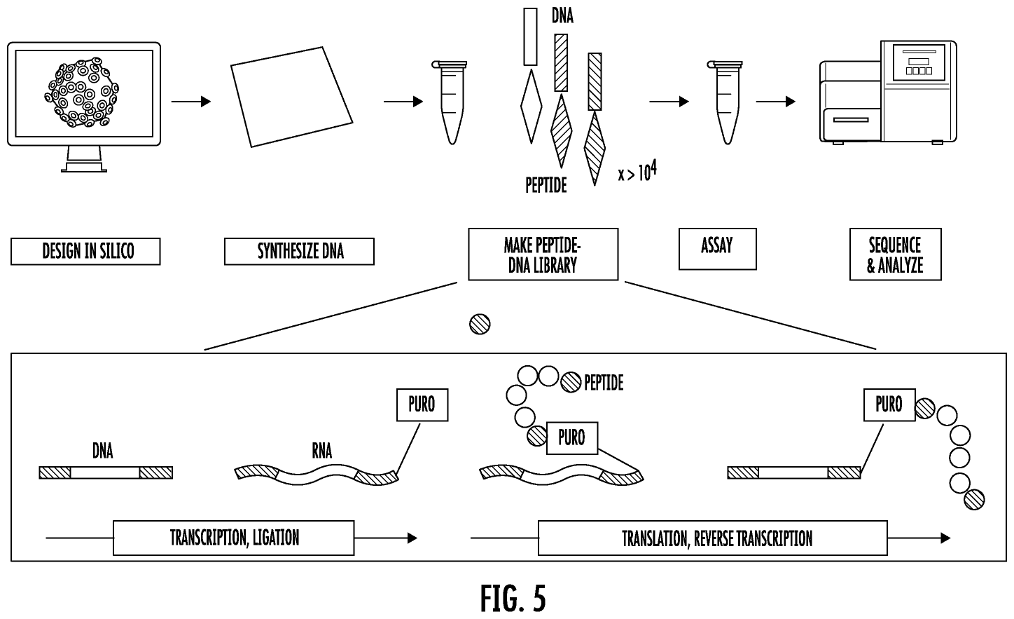

The PepSeq platform provides a method for efficient synthesis and analysis of large libraries of DNA-barcoded peptides (see FIG. 5). As part of the NeoTIL approach, the Inventors have also developed methods for growing tumor organoids that provide a personalized test-bed for the anti-tumor efficacy of the cellular product.

The majority of procedures for this approach are interrelated. The Inventors have received IRB approval and have already enrolled a range of solid tumor patients onto a clinical trial in this area, which include the elements of the inventive approach related the DNA/RNA sequencing and organoid isolation growth and testing as detailed further below.

### Example 2

### A Method for the Identification and Expansion of Neoantigen-Specific TILs from Human Tumors

The following methods enable the identification and isolation of neoantigen-specific TILs from human tumors. Freshly-resected tumor specimens from human patients with various malignancies will each be split into 2 samples. The first sample will be used to extract DNA and RNA, enabling whole exome sequencing and RNAseq. Together with a germline sample (e.g., blood or cheek swab), this will enable the identification of somatic variants and quantification of the expression of the associated proteins.

Following analysis of the sequencing data, the tumor genomic information will be used to design libraries of overlapping peptides covering candidate mutations and their wild-type counterparts. DNA-encoded libraries of these peptides will be synthesized in a highly-parallel fashion using the PepSeq platform or a phage display platform, for example, and assayed in parallel against a panel of commonly-expressed human MHC class II proteins. In parallel, in silico models of MHC class I binding will be applied to identify a second subset of candidate neoantigens. The resulting data will enable prioritization of a subset of putative class I or II-restricted neoantigens for downstream attention.

The second tumor sample will be dissociated into single cells using standard methods. Using the candidate neoantigens identified by the PepSeq or a phage display platform and/or in silico prediction approach, two parallel approaches for enriching neoantigen-reactive T cells from this sample will be pursued. In the first approach, selective expansion of neoantigen-reactive T cells will be accomplished by culturing dissociated tumor samples with peptides representing the shortlisted (i.e., prioritized) neoantigen sequences, in the presence of cytokines. In the alternative, fluorescently-labeled peptide:MHC probes corresponding to the shortlisted sequences (and perhaps in combination with cellular exhaustion markers) will be used to isolate neoantigen-reactive T cells by fluorescence-activated cell sorting. In each case, in vitro activation assays will be used to measure the activity of the cellular product against autologous tumor cells, including possibly the use of in vitro-derived tumor organoids.

### Example 3

### An Alternative Method for the Identification and Expansion of Neoantigen-Specific TILs from Human Tumors

Freshly-resected tumor specimens from human patients with various malignancies are split into 2 samples. The first sample is used to extract DNA and RNA, enabling whole exome sequencing and RNAseq. Together with a germline sample (blood or cheek swab), this enables the identification of somatic variants and quantification of the expression of the associated proteins.

Following analysis of the sequencing data, the tumor genomic information is used to design libraries of overlapping peptides covering candidate mutations and their wild-type counterparts. DNA-encoded libraries of these peptides is synthesized in a highly-parallel fashion using the PepSeq platform, and assayed in parallel against a MHC class II (DR, DP, and DQ) specific for the patient. This process enables the rapid, high-confidence identification of candidate neoantigen peptide:MHCs by selectivity and by affinity. As a result, a small set of targeted neoantigens from among the typically large catalog of peptide-changing somatic tumor variants is selected.

The second tumor sample is dissociated into single cells using standard methods, and processed to isolate TILs and tumor cells. To address the limitation of the non-specific in vitro T cell expansion, TILs are sub-cultured in the presence of the candidate neoantigens peptides identified by the PepSeq. This approach enables preferential expansion of cells of the desired specificity.

This approach ensures the generation of a neoantigen peptides-specific enriched TIL population that can be tested in an immune infiltration assay performed on patient-derived tumor organoids. Upon T cell enrichment, it is possible to use single cell sequencing, to identify the paired rearranged TCRα and β genes that confer T cell neoantigen specificity. This can provide an additional metric of the efficiency of T cell enrichment, and also enable a parallel gene transfer approach in which patient-specific neoantigen reactivity is conferred on universal donor cells, offering the potential to circumvent the exhausted phenotype of the endogenous response.

To be clear, the entire lymphocyte population is generally used after enrichment via incubation with the mixture of peptides (i.e., neoantigens).

To test the efficiency of the approach, immune infiltration assays are performed on patient-derived tumor organoids. Briefly, tumor cells isolated from the original specimen are cultured in ultra low affinity (ULA) plate to form 3D structure (organoids). Organoids are known to recapitulate the phenotypic characteristic of the tissue of origin, including the antigenic potential. Following co-culture of neoantigen peptides-specific TILs with tumor organoids derived from the same patient, the level of TIL infiltration into the organoid is quantified by z-stacking imaging.

Neoantigen peptides-specific TILs can be expanded in vitro in presence of cytokines and then reinfused into the patient, typically following a lymphodepleting conditioning regimen.

### Example 4

### Collection of TILs from Human Patients and Mice

Several human patients enrolled in a clinical trial to test the efficacy of the NeoTIL therapy. The patients had been diagnosed with lung cancer, colorectal cancer, melanoma, breast cancer, and colon cancer. In addition, a BALB/c mouse model of colon cancer was used for the collection of TILs.

Tumor biopsies were collected from each human patient and from the mice. The numbers of isolated tumor cells (TCs), peripheral blood mononuclear cells (PBMCs), and TILs were determined for each human patient (see Table 1). In addition, the MHC alleles and number of mutations were identified for each human patient and for the BALB/c mouse model of colon cancer (see Table 2).

TABLE 1
Collection of TILs from tumor biopsies removed from human patients.

| | | Biopsy | Tissue | | | |
|---|---|---|---|---|---|---|
| Patient ID | Cancer | Origin | (mg) | TCs | PBMCs | TILs |
| TG0006 | lung | surgery - lung | ~1.0 gm- | 2.1 × 10⁶ | 7.8 × 10⁶ | 3.04 × 10⁶ |
| | | | tumor | | | |
| TG00013 | melanoma | lump (excised | 1.1 gm- | 2.65 × 10⁶ | 3 × 10⁶ | 7.5 × 10⁵ |
| | | from back) | tumor | | | |
| TG00024 | metastatic | left anterior | ~2.5 gm | 3.89 × 10⁶ | 10 × 10⁶ | 6.70 × 10⁵ |
| | melanoma | thigh excisional | tumor | | | |
| | | biopsy | | | | |

TABLE 2
Identification of MHC alleles and mutations for each human
patient and for the BALB/c mouse model of colon cancer.

| | | | Biopsy | | | Number of |
|---|---|---|---|---|---|---|
| Species | Patient ID | Cancer | Site | MHC Alleles | | Mutations |
| human | TG00006 | primary lung | lung | DRB1*07:01 | DRB1*11:03 | 83 |
| human | TG00013 | metastatic | back | DRB1*03:01 | DRB1*01:02 | 374 |
| | | melanoma | | | | |

| | | | | | |
|---|---|---|---|---|---|
| mouse | BALB/c | colon cancer | N/A | 1(A)d | 1506 |
| | Mouse | model | | | |

### Example 5

### Exome Sequencing, Peptide Library Design, and Binding Assays with Human Samples

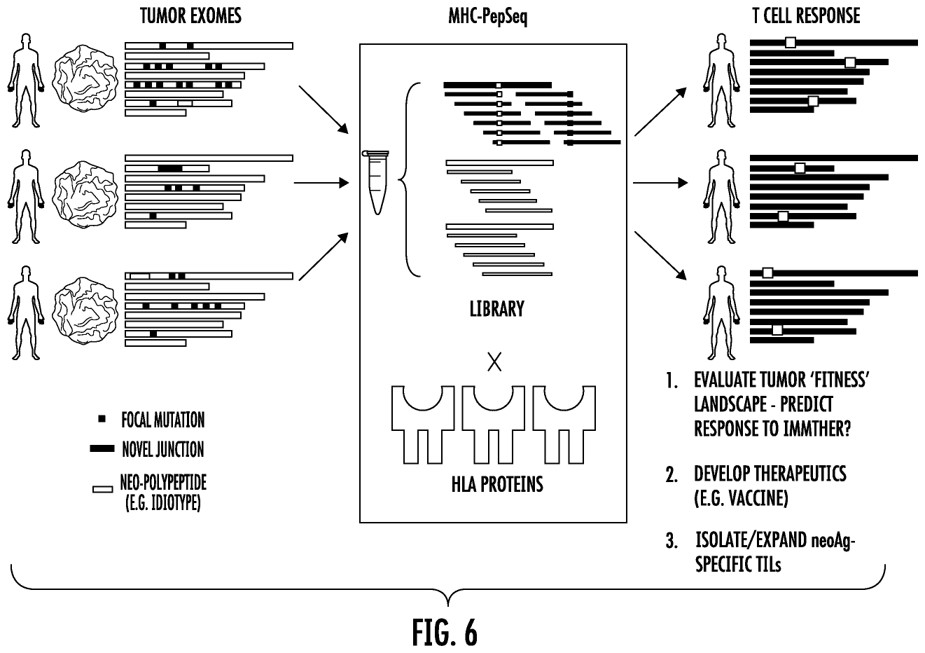

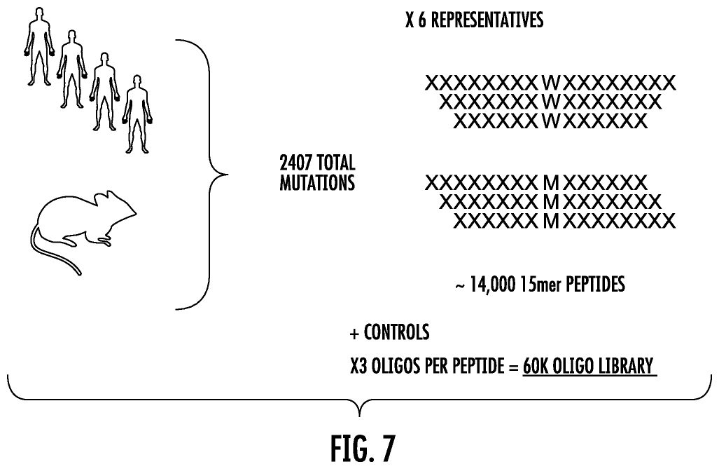

Whole exome sequencing of tumor exomes and exomes from normal cells was used to design a PepSeq library as outlined in FIGS. 6 and 7. Peptides in the PepSeq library overlapped in sequence and contained wild-type residues (“W”) and mutant residues (“M”) (see FIG. 7).

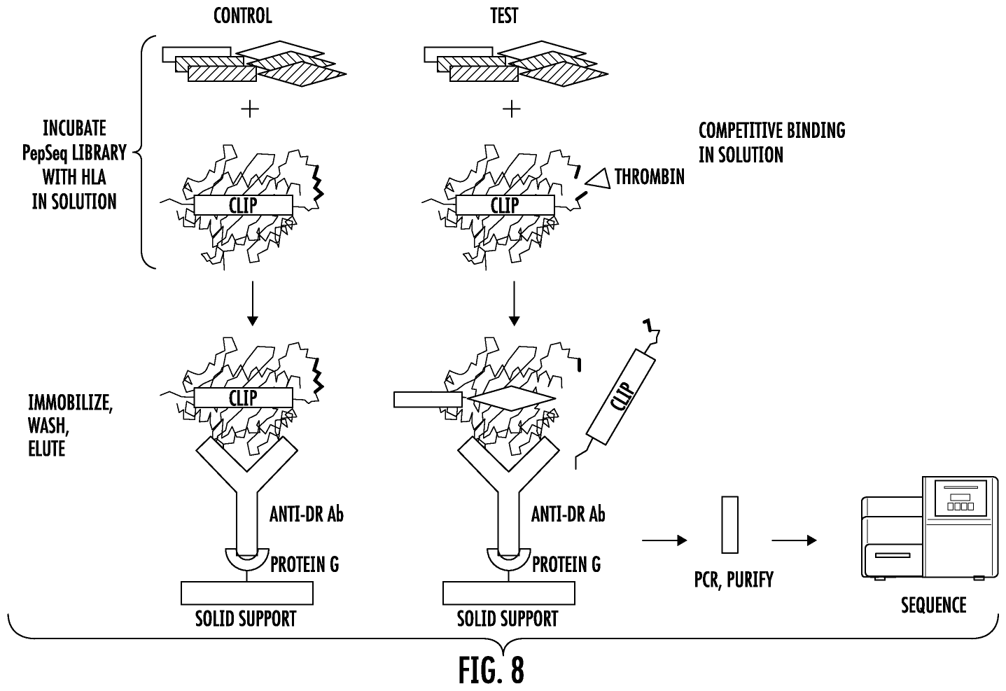

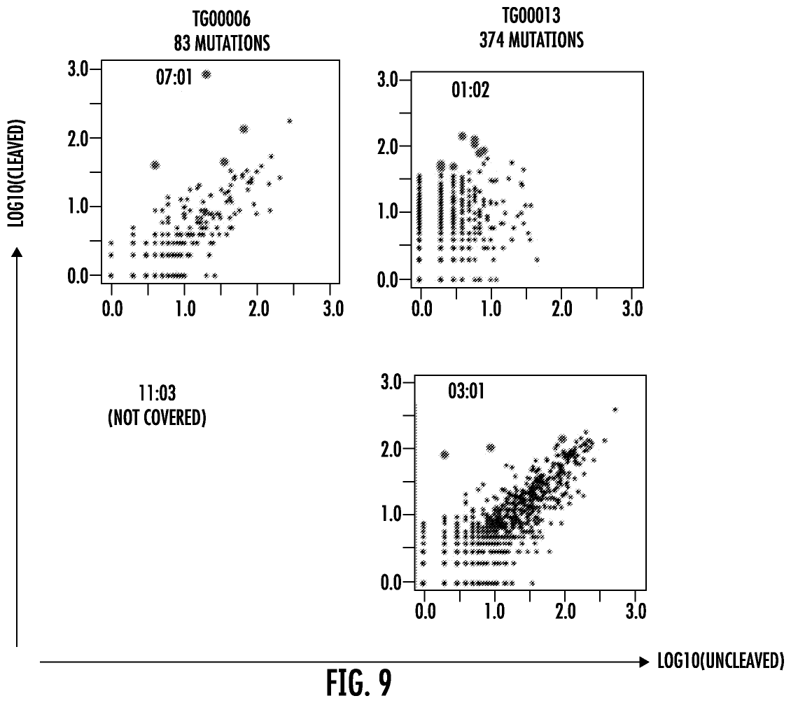

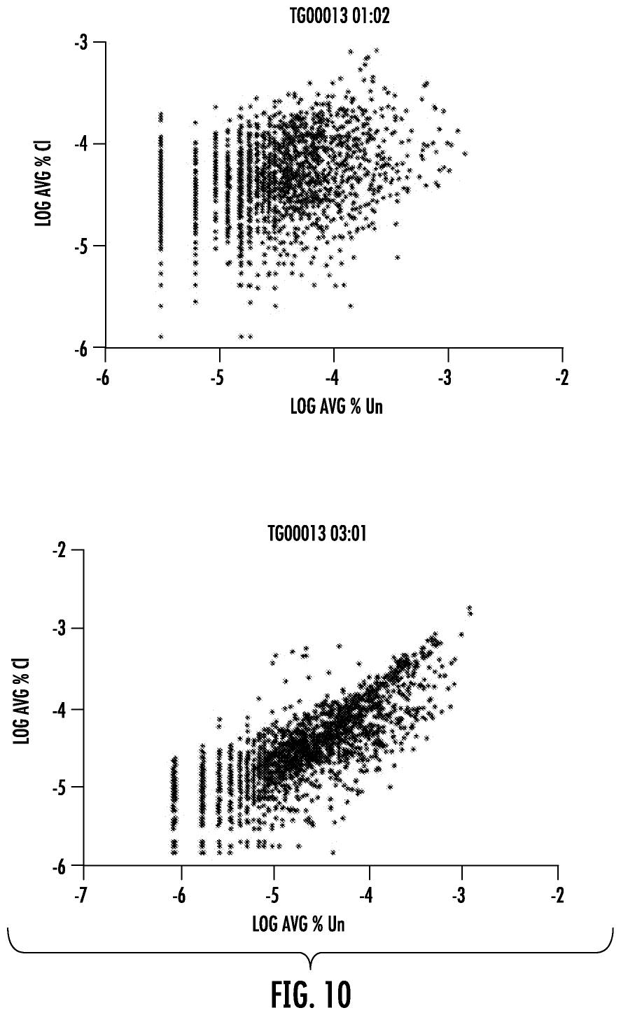

For human patients TG00006 and TG00013 a binding assay was performed as shown in FIG. 8 using the methods outlined in Day et al., J Clin Invest. 2003; 112(6):831-842. The binding assay identified peptides with high affinity for the particular human leukocyte antigen serotypes present in each patient (see the larger data points in the upper left-hand corner of each graph shown in FIG. 9). Additional analyses with the binding assay using peptide libraries for human patient TG00013 are shown in FIG. 10.

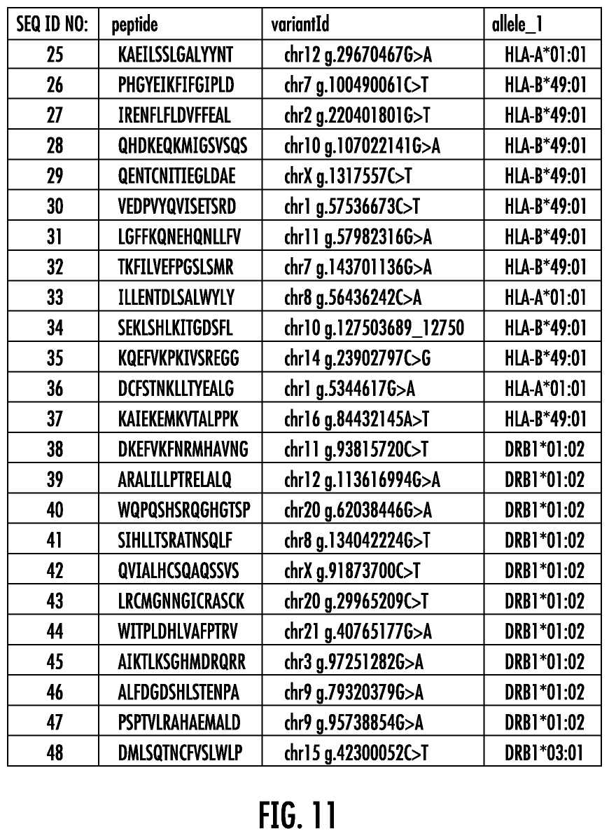

The nucleic acids encoding the peptides with high affinity for the HLA complexes were isolated with the PepSeq peptide constructs. These nucleic acids were sequenced and the corresponding amino acid sequences are presented in FIG. 11 for human patient TG00013.

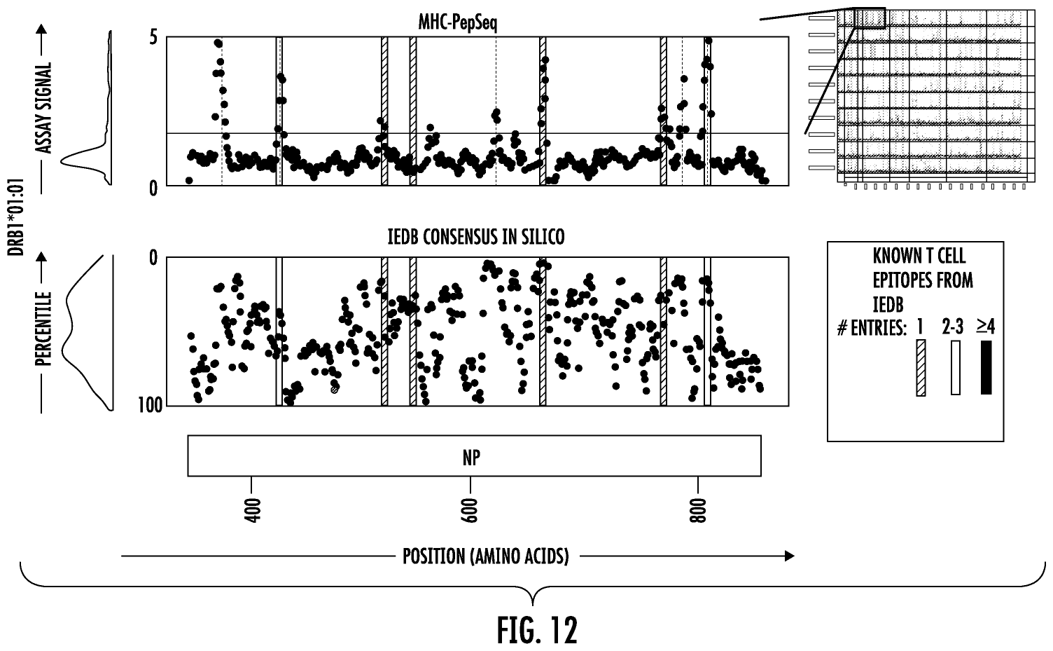

Comparison of MHC-PepSeq results with in silico predictions can be used to identify and confirm neoantigens and/or neoepitopes as shown in FIG. 12.

### Example 6

### Infiltration Assays with Human Samples

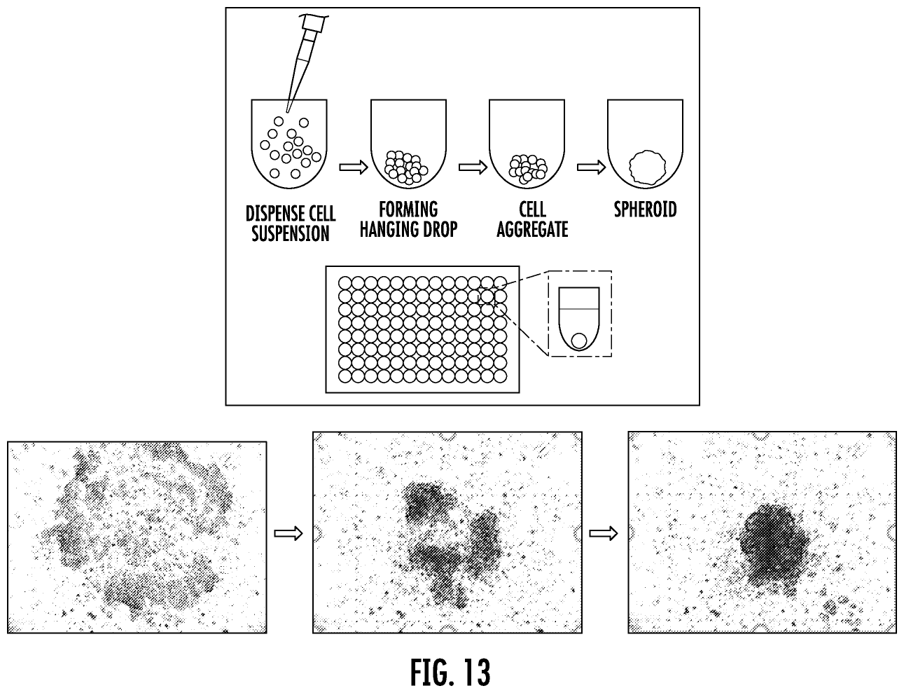

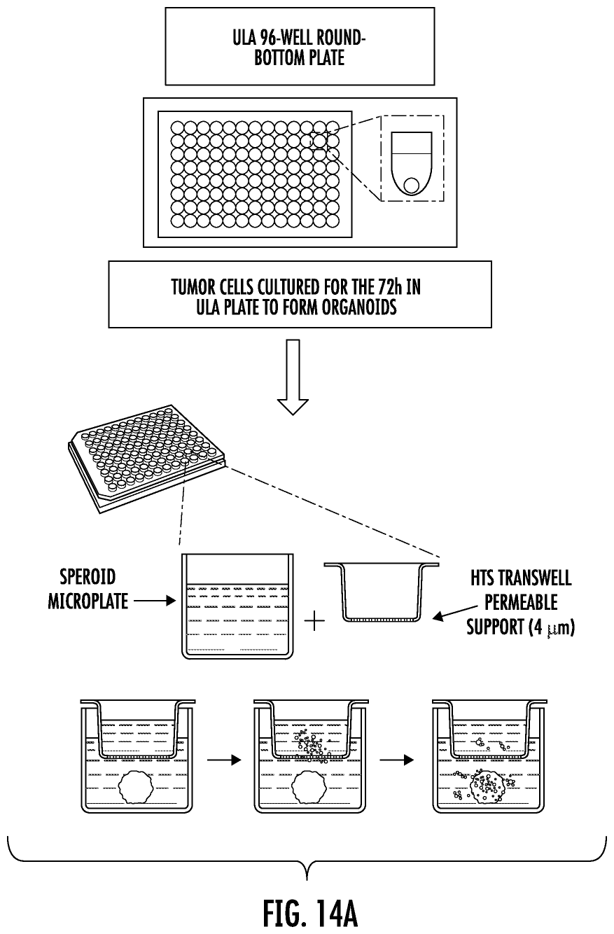

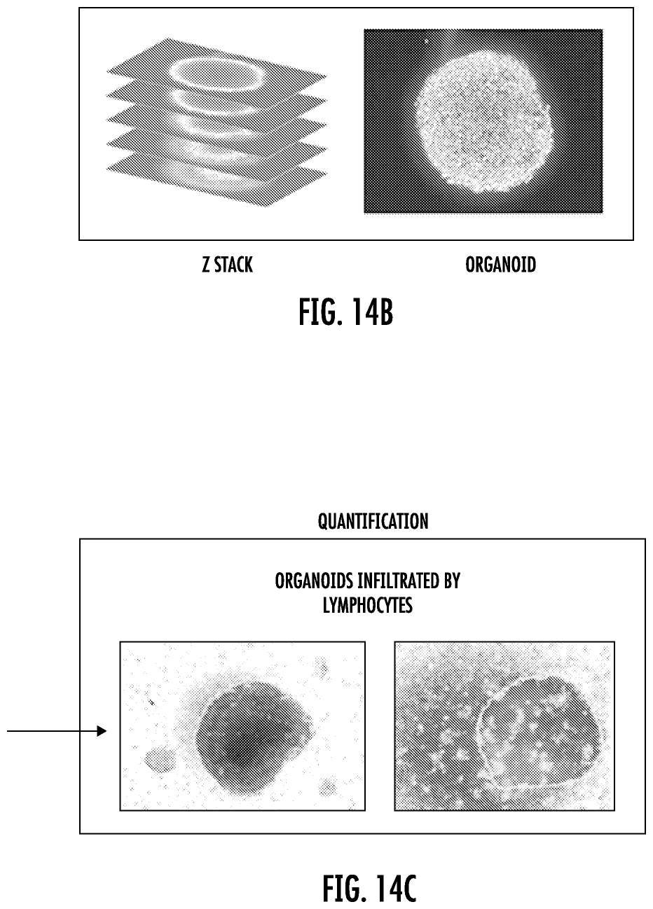

Organoids were formed from tumor cell suspensions in a round-bottom ultra low affinity (ULA) plate, a permeable support was inserted, and enriched TILs were allowed to migrate through the support (i.e., transwell insert) to infiltrate the organoids as shown in FIG. 13 and FIG. 14A. Three-dimensional images of the organoids were generated with Z-stacking, and quantification of infiltration of the lymphocytes into the organoids was performed as shown in FIGS. 14B and 14C.

Enriched TILs were stained with Invitrogen CELLTRACKER™ CM-DiI, a red fluorescent dye well suited for monitoring multigenerational cell movement or location to facilitate the quantification of TILs infiltration into the organoids. The following treatments were administered to the organoids: 1) TILs; 2) TILs+IL-2; 3) TILs+IL-2+peptides (i.e., neoantigens); and 4) TILs+peptides (i.e., neoantigens). TIL infiltration into the organoid was quantified 6 hr, 12 hr, 24, hr, 48 hr, and 120 hr after administration.

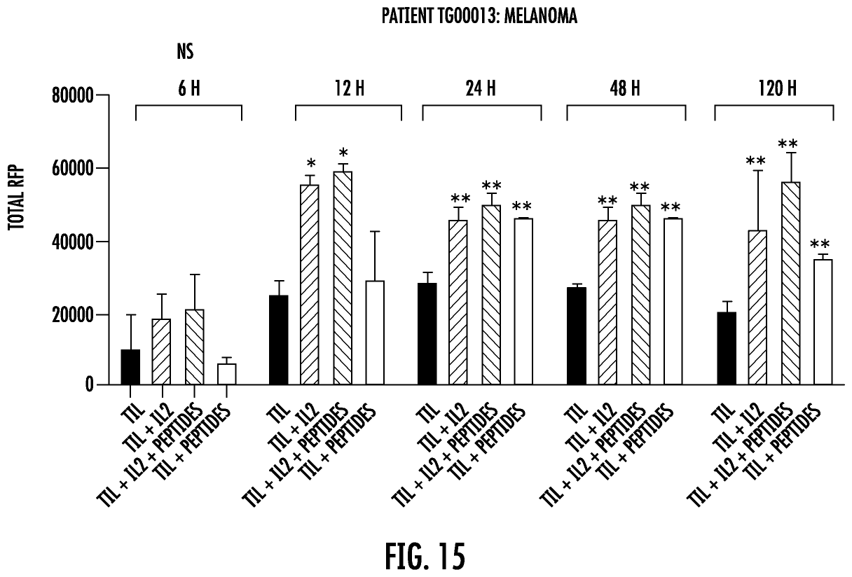

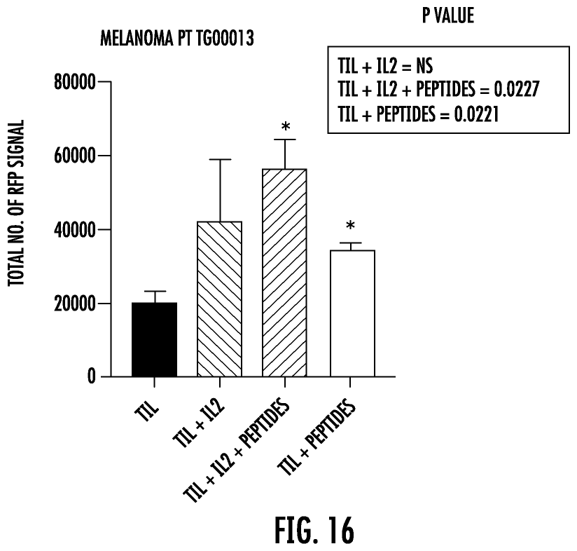

TIL infiltration into the organoids was significantly increased with TILs exposed to IL-2 and/or peptides (i.e., neoantigens) with the greatest infiltration generally occurring with TILs exposed to both IL-2 and peptides (see FIGS. 15 and 16).

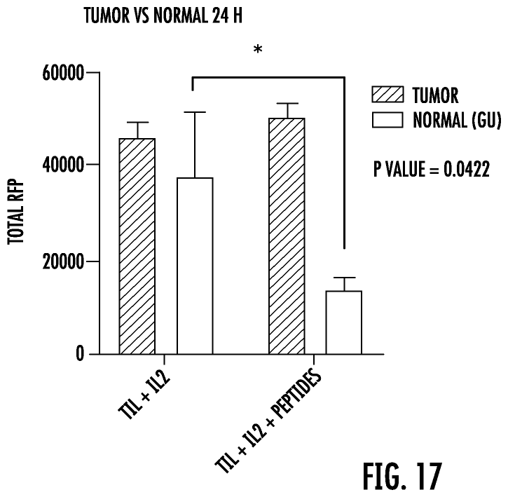

The Inventors compared neoantigen peptides-specific TIL infiltration in tumor versus normal tissue obtained from the same patient. Importantly, while TILs treated with IL-2 actively infiltrate tumor and normal tissue, neoantigen peptides-specific TILs fail to recognize normal tissue, confirming the tumor selectivity insured by the NeoTIL approach (see FIG. 17).

### Example 7

### NeoTIL Analysis and Therapy with a CT26 Colon Carcinoma Mouse

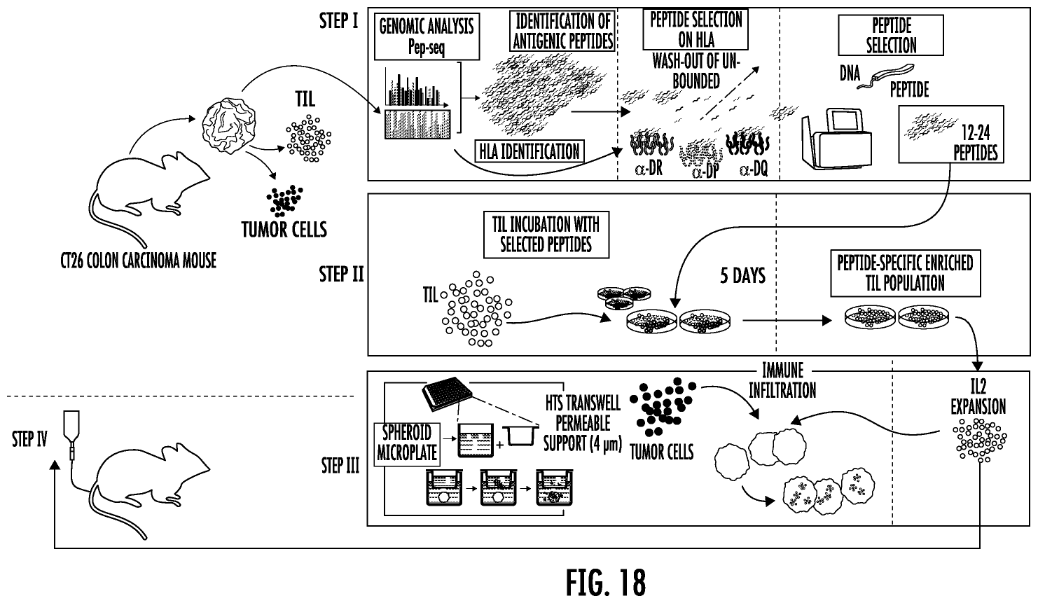

The work flow for analyzing and treating a CT26 colon carcinoma mouse with NeoTIL is outlined in FIG. 18. Exome sequencing, peptide library design, and binding assays were performed as described in Example 5 with the CT26 colon carcinoma mouse.

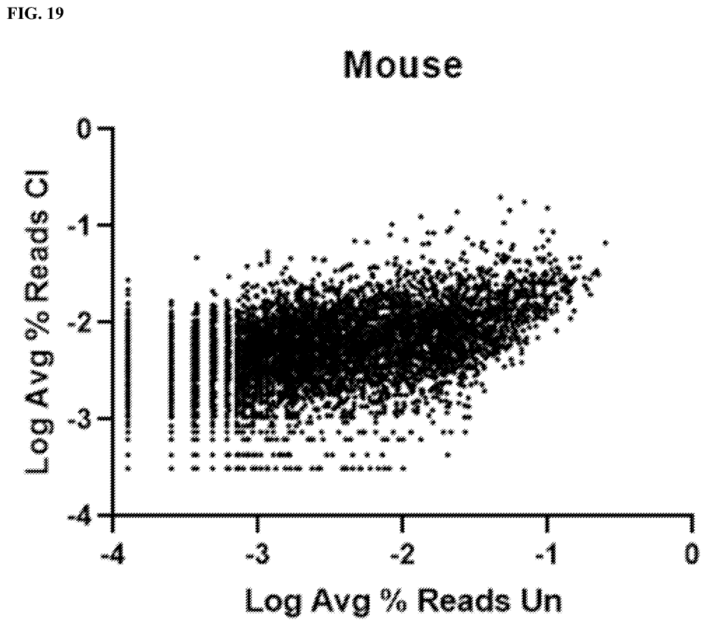

The binding assay identified peptides with high affinity for the particular mouse MHC class I or class II alloantigens present in the CT26 colon carcinoma mouse (see the data points in the upper portion of the graph shown in FIG. 19).

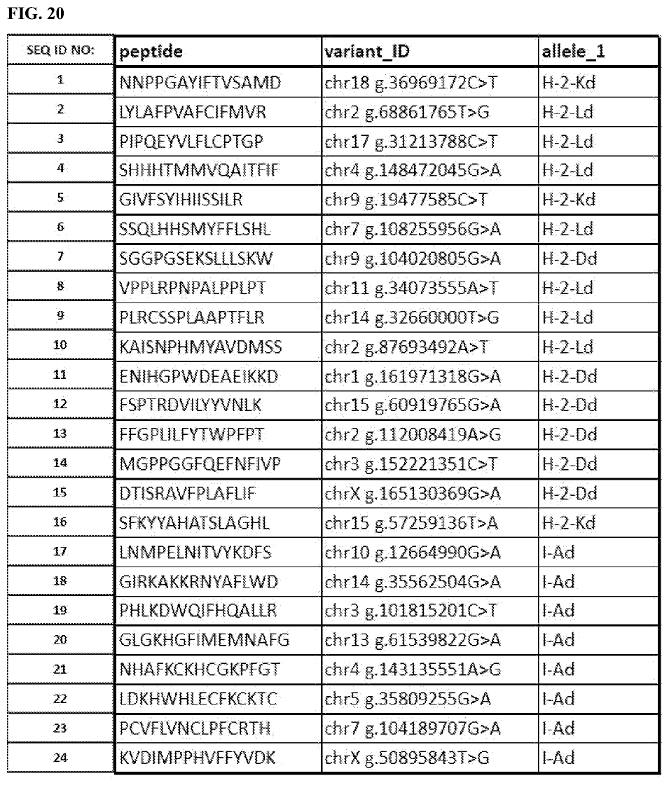

The nucleic acids encoding the peptides with high affinity for the mouse MHC class I or class II alloantigens were isolated with the PepSeq peptide constructs. These nucleic acids were sequenced and the corresponding amino acid sequences are presented in FIG. 20.

### Example 8

### Infiltration Assays and Tumor Growth Assays with the CT26 Colon Carcinoma Mouse

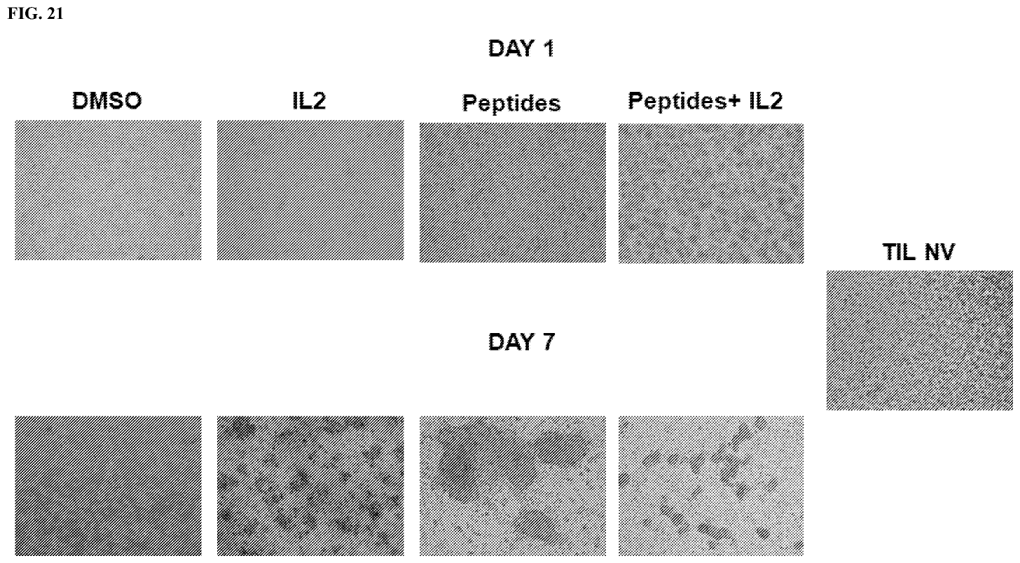

Infiltration assays with organoids derived from CT26 colon carcinoma mouse tumor cells were performed as described in Example 6. Microscopic images of TILs co-cultured with DMSO (i.e., peptide vehicle), IL-2, peptides (i.e., neoantigens), or peptides with IL-2 at Day 1 and at Day 7 are shown in FIG. 21.

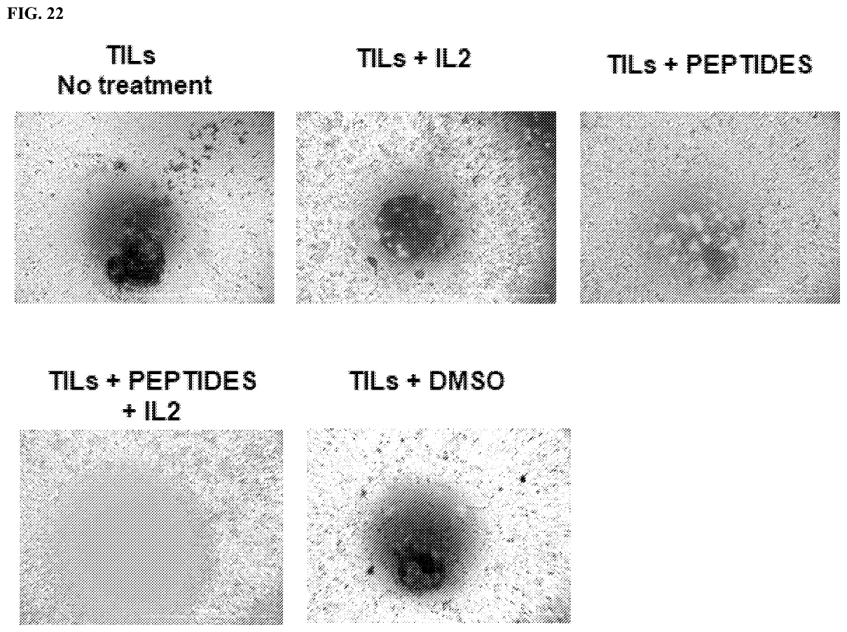

In this mouse model, co-cultures of TILs with tumor organoids in different conditions (i.e., TIL with no treatment, TIL+IL-2, TIL+peptides, TIL+IL-2+peptides) showed that incubation of TILs with the combined peptides and IL-2 results in the highest and statistically significant infiltration level in tumor organoids (P<0.0001) (see FIGS. 22 and 23).

Tumor volumes were measured in CT26 colon carcinoma mice after intratumoral (“IT”) administration or intravenous (“IV”) administration of different TIL treatments. No lymphodepletion or IL-2 (other than that present in certain expanded TIL populations) were used with the mice. For IT administration, treatments included 1) TILs; 2) TILs+IL-2; 3) TILs+peptides (i.e., neoantigens); 4) TILs+IL-2+peptides (i.e., neoantigens); and 5) vehicle. For IV administration, treatments included 1) TILs+IL-2; 2) TILs+peptides (i.e., neoantigens); 3) TILs+IL-2+peptides (i.e., neoantigens); and 4) TILs+vehicle.

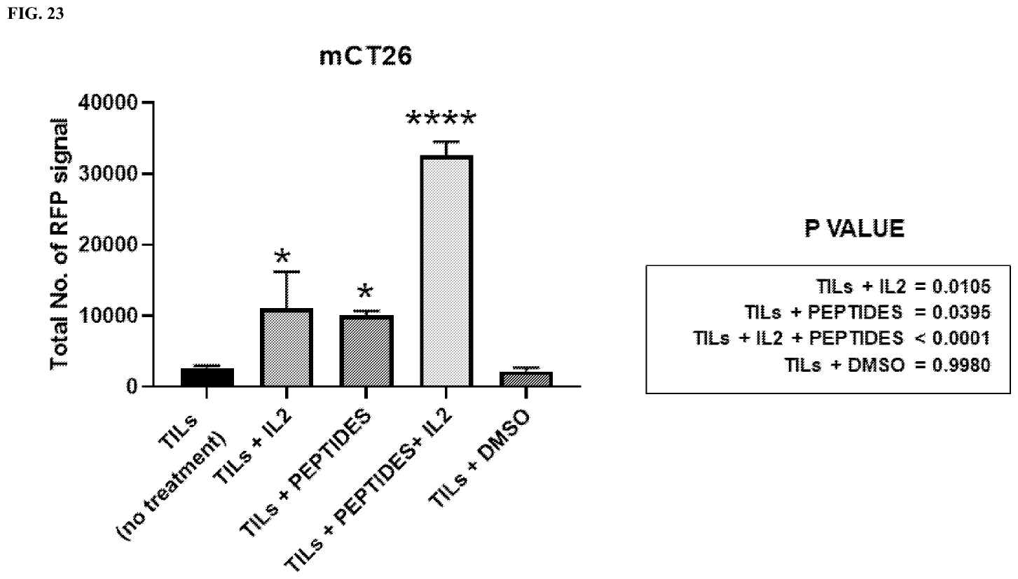

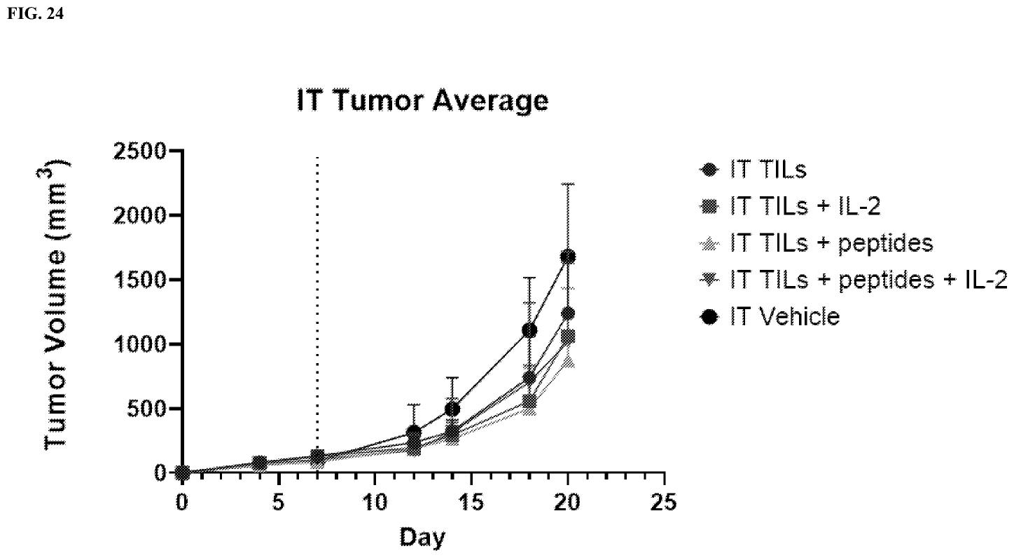

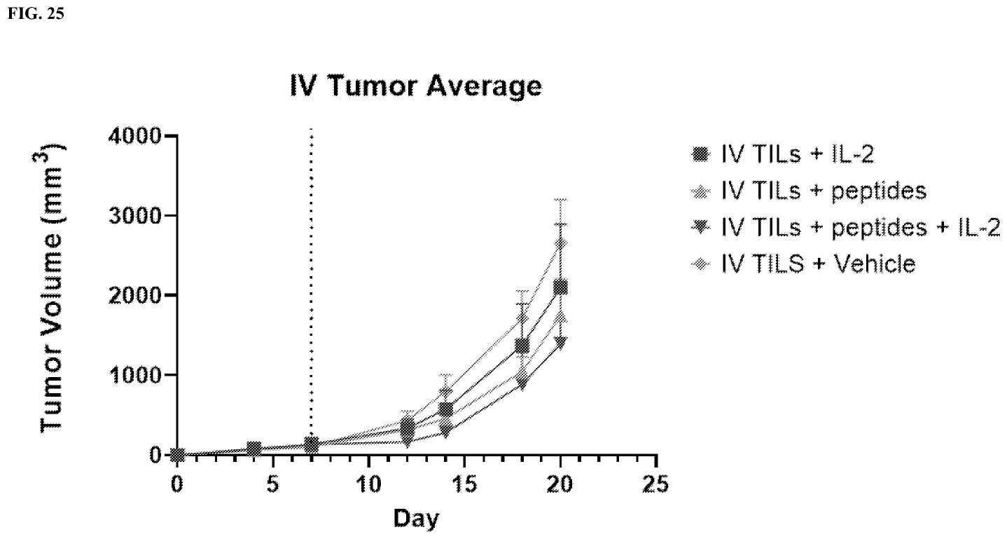

These in vivo studies with the CT26 colon carcinoma mouse model show that, even in absence of lymphodepletion conditioning, neoantigen peptides-specific TILs promote an approximate 50% reduction in tumor volume (see FIGS. 24 and 25).

### Example 9

### Using PepSeq to Identify the Neoantigens for Training TILs

Peptides are identified as putative Class II MHC binders using PepSeq. Raw sequence reads are aligned to nucleotide encoded reference sequences of the PepSeq library to produce a matrix of raw read counts for each MHC sample and reads per peptide. PepSeq binding score ratio is calculated by taking the ratio of normalized binding signal for each MHC II sample for each peptide with respect to its paired negative control (uncleaved MHC). A constant-adjusted ratio threshold is applied to binding scores to select peptides for inclusion in TIL+peptide cell culture experiments.

In silico predictions are used for selecting peptides with binding potential to Class I MHC. In silico predictions are made using publicly available tools for unique 9mers tiled across the PepSeq library. Peptides with minimum predicted MHC binding percentile rank (lower rank corresponds to stronger binding) with respect to a random set of peptides are selected for inclusion in the TIL+peptide cell culture. Predictions are generated for each of the patients Class I MHC alleles (up to six alleles).

A custom pool of peptides is selected using the described approaches for Class I and Class II MHC coverage for each patient with roughly half composed of the best Class II binding peptides and half of the best Class I binding peptides for a total of approximately 24 peptides in the final pool. Experimental factors may shift the composition of the pool slightly for Class I or Class II.

### Example 10

### Neo-TIL Efficacy in Melanoma, Lung Cancer, Colon Cancer, and Pancreatic Cancer

Tumor samples were dissociated into single cells using standard methods and processed to isolate TILs. TILs were sub-cultured in the presence of the candidate neoantigens peptides identified by the PepSeq. This approach enables preferential expansion of cells of the desired specificity. TILs were rapidly expanded using non-specific T-cell receptor stimulation by administering mouse monoclonal anti-CD3 antibody OKT3 in the presence of lymphocytes and IL-2.

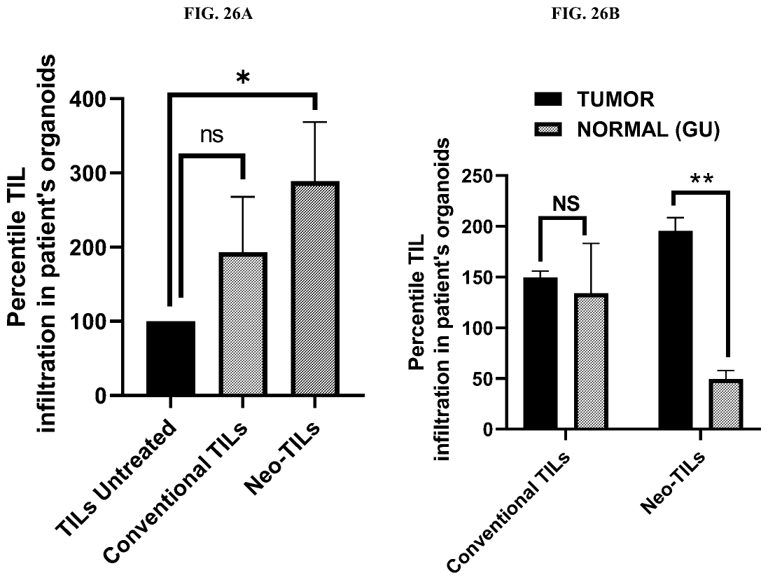

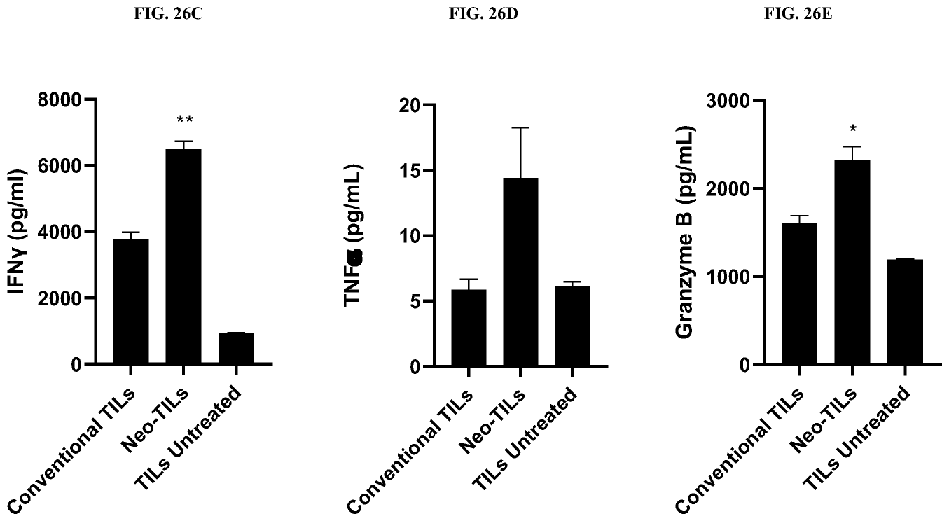

Organoids were formed from tumor cell suspensions in a round-bottom ultra-low affinity (ULA) plate, a permeable support was inserted, and enriched TILs were allowed to migrate through the support (transwell insert) to infiltrate the organoids. Enriched TILs were stained with Invitrogen Vybrant™ CM-Dil, a red fluorescent dye well suited for monitoring multigenerational cell movement or location to facilitate the quantification of TIL infiltration into the organoids. Three-dimensional images of the organoids were generated with Z-stacking, and infiltration of the lymphocytes into the organoids was quantified (FIGS. 26A, 26B, 27A, 28A, 28B, 29A, 30A, 31A-31C, and 32).

The following treatments were administered to the organoids: 1) untreated TILs; 2) TILs+IL-2 (i.e., conventional TILs); and 3) TILs+peptides (i.e., Neo-TILs). TIL infiltration into the organoid was quantified 24 hr after administration.

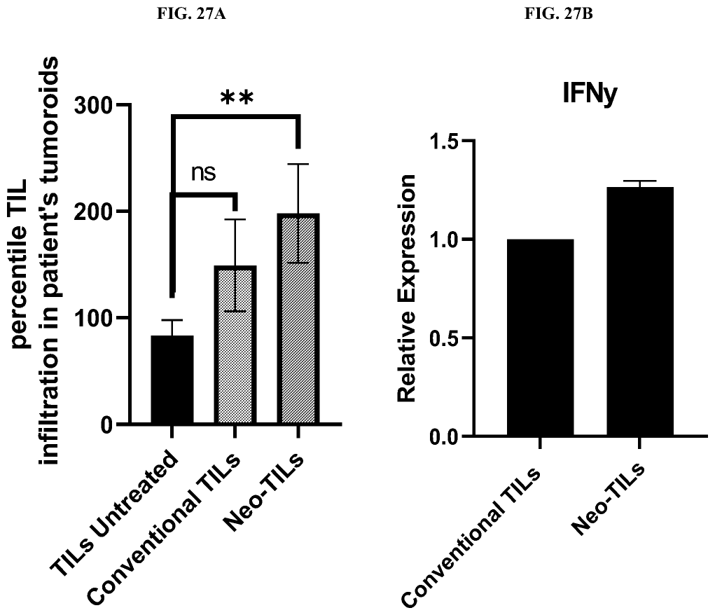

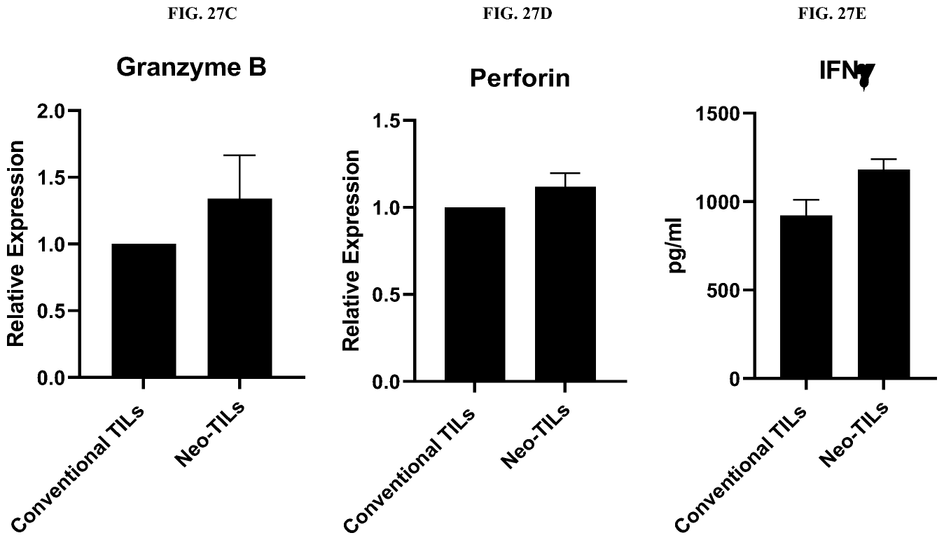

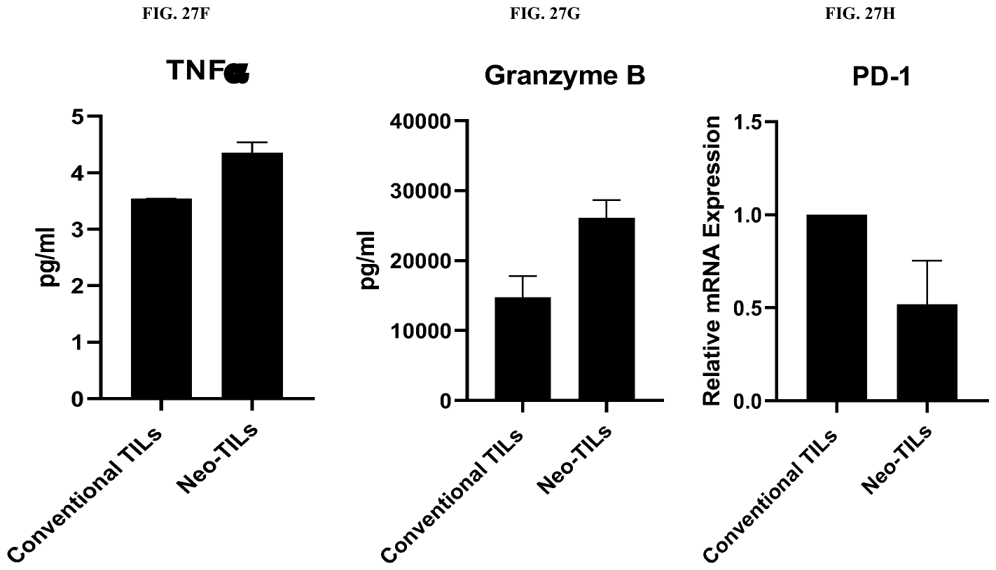

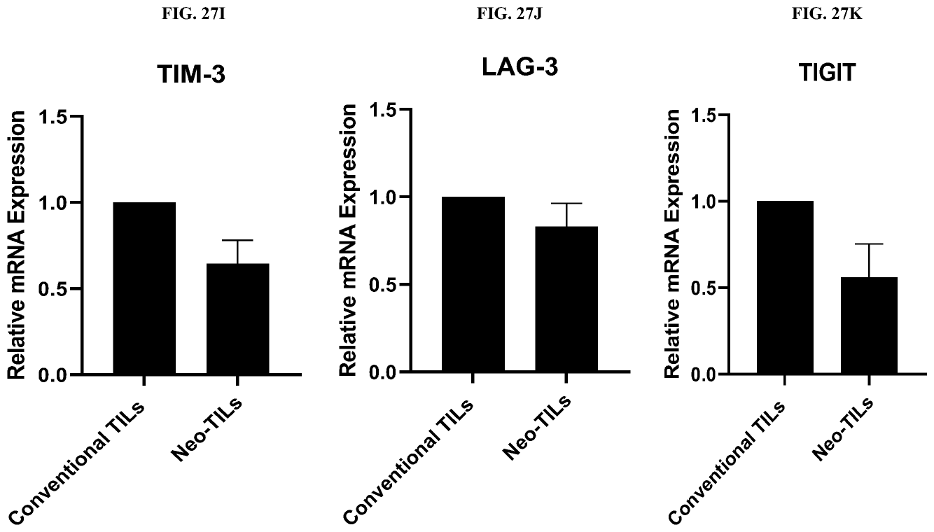

TIL infiltration into the melanoma patient's tumor organoids was significantly increased with TILs exposed to peptides (i.e., Neo-TILs) relative to untreated TILs (see FIGS. 26A, 27A). Importantly, while conventional TILs actively infiltrate tumor and normal tissue, neoantigen peptides-specific TILs (i.e., Neo-TILs) fail to recognize normal tissue, confirming the tumor selectivity insured by the Neo-TIL approach (see FIG. 26B).

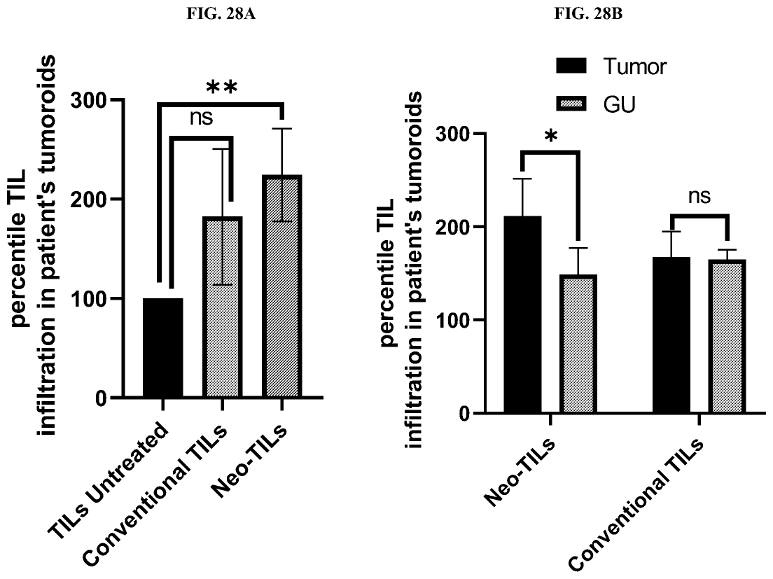

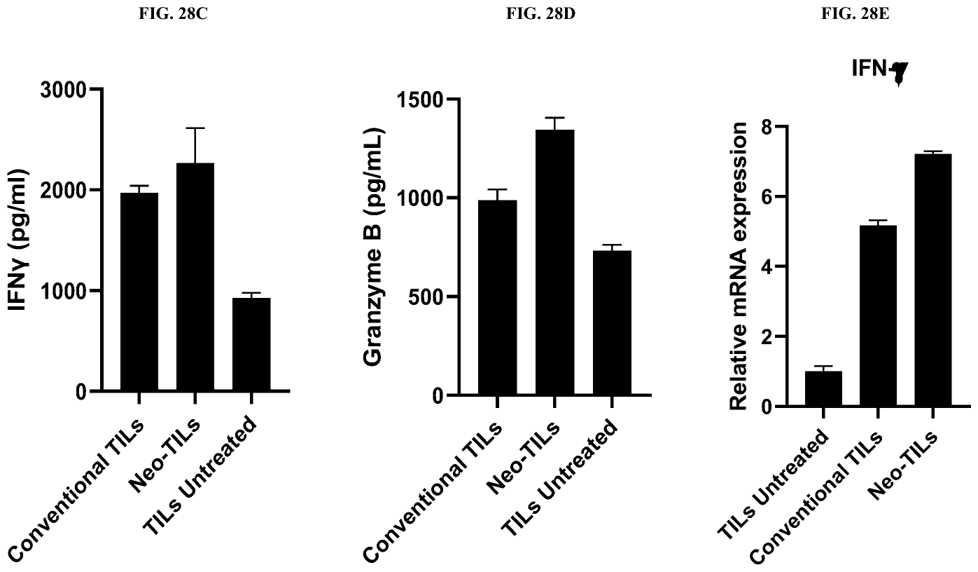

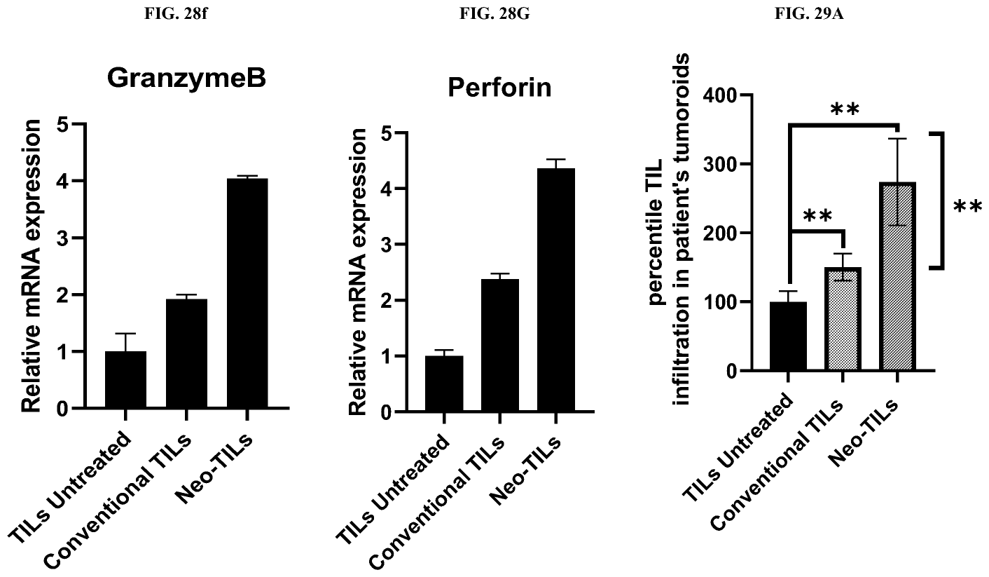

TIL infiltration into the lung cancer patient's tumor organoids was significantly increased with TILs exposed to peptides (i.e., Neo-TILs) relative to untreated TILs (see FIG. 28A). Additionally, conventional TILs actively infiltrate tumor and normal tissue, whereas neoantigen peptides-specific TILs (i.e., Neo-TILs) recognized selectively tumor tissue (see FIG. 28B).

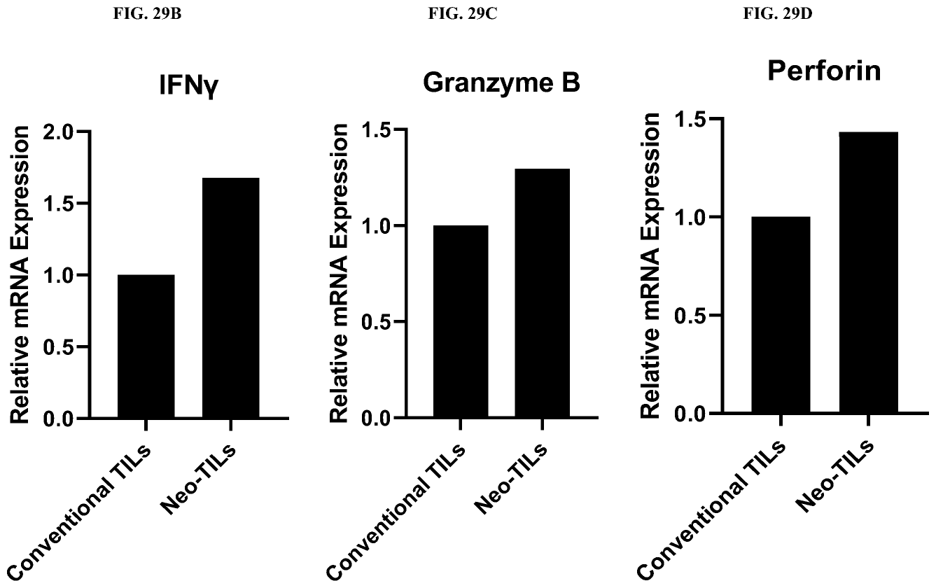

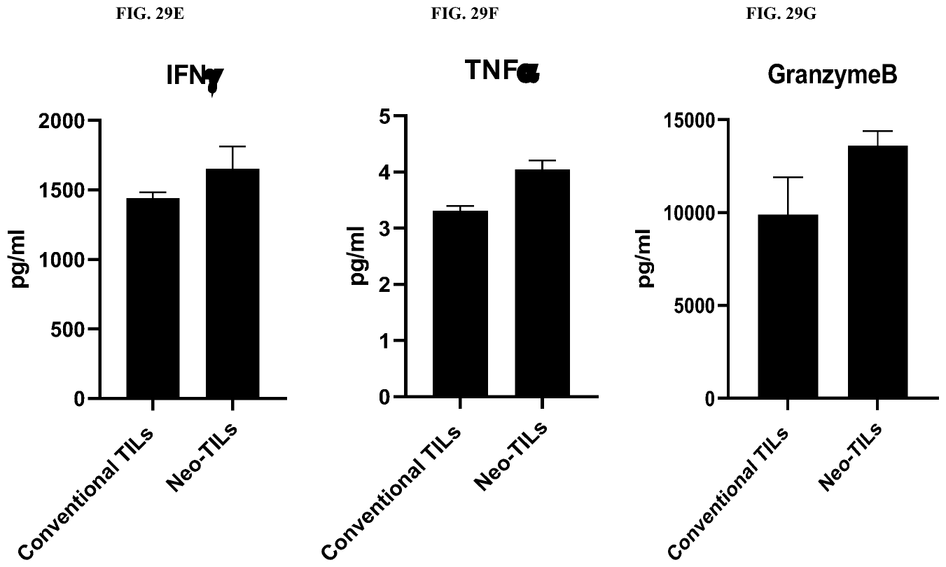

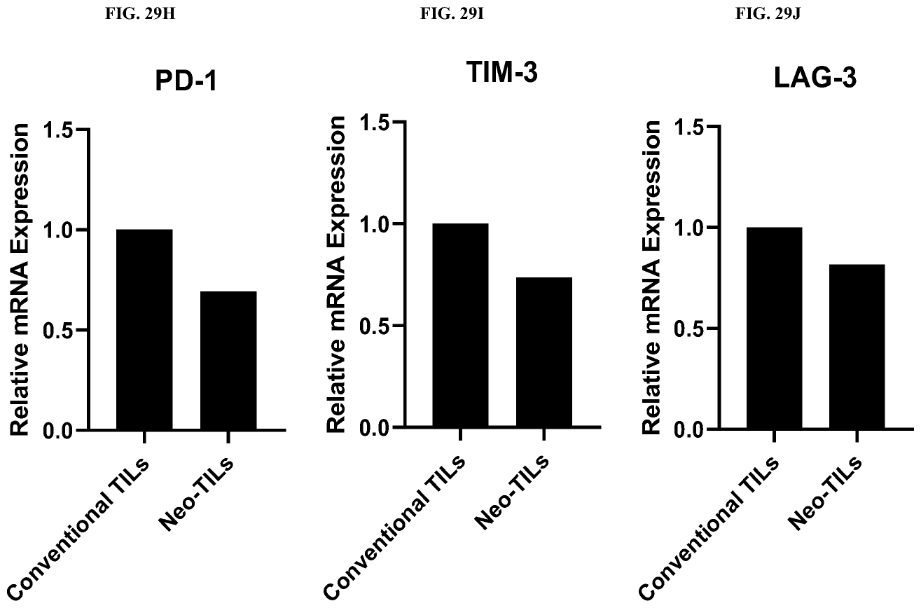

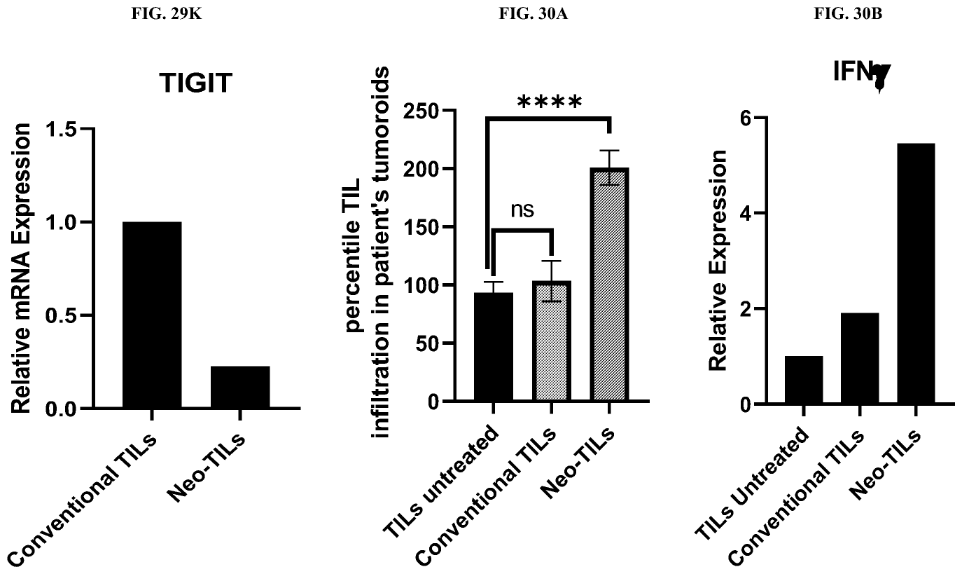

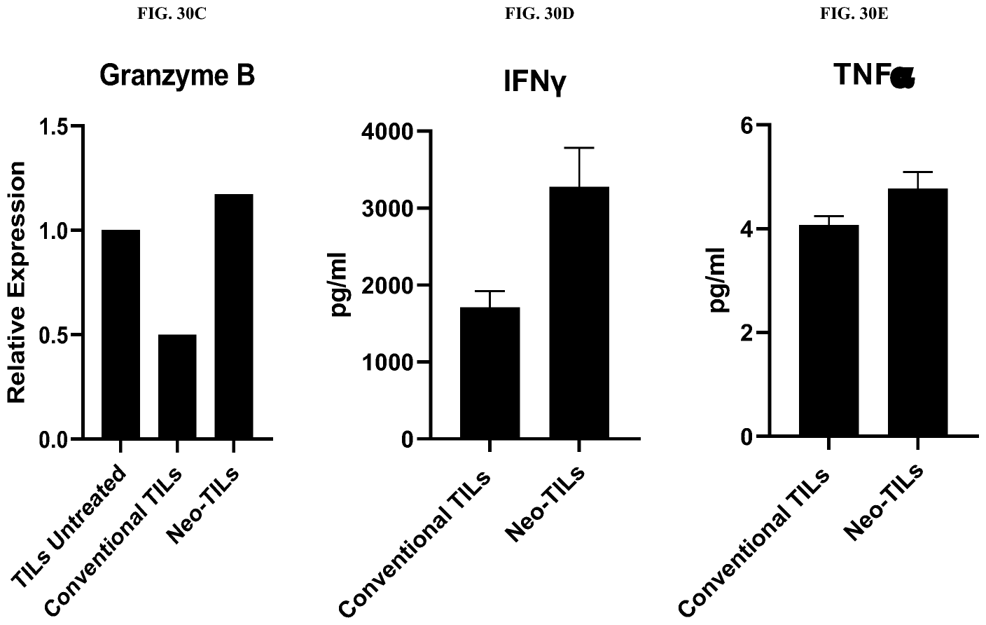

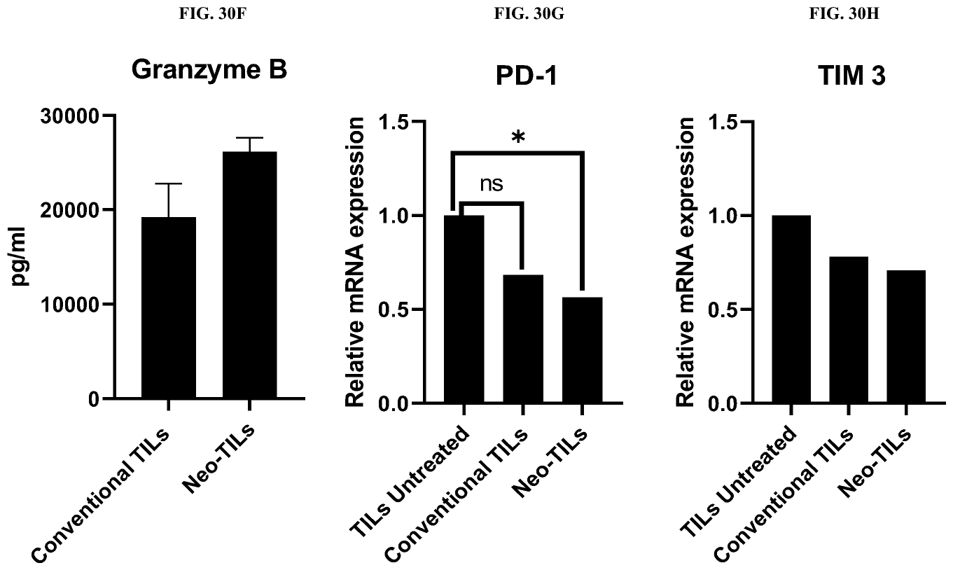

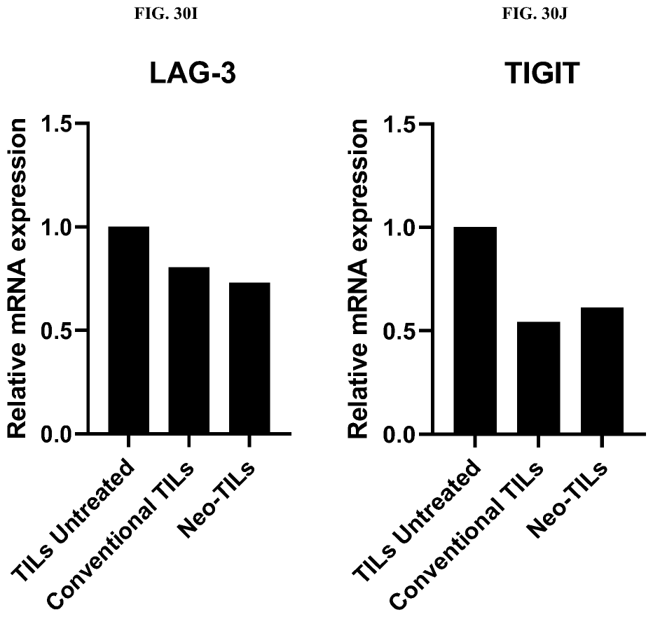

Further, TIL infiltration into the colon cancer patient's tumor organoids and pancreatic cancer patient's tumor organoids was significantly increased with TILs exposed to peptides (i.e., Neo-TILs) relative to untreated TILs (see FIGS. 29A and 30A).

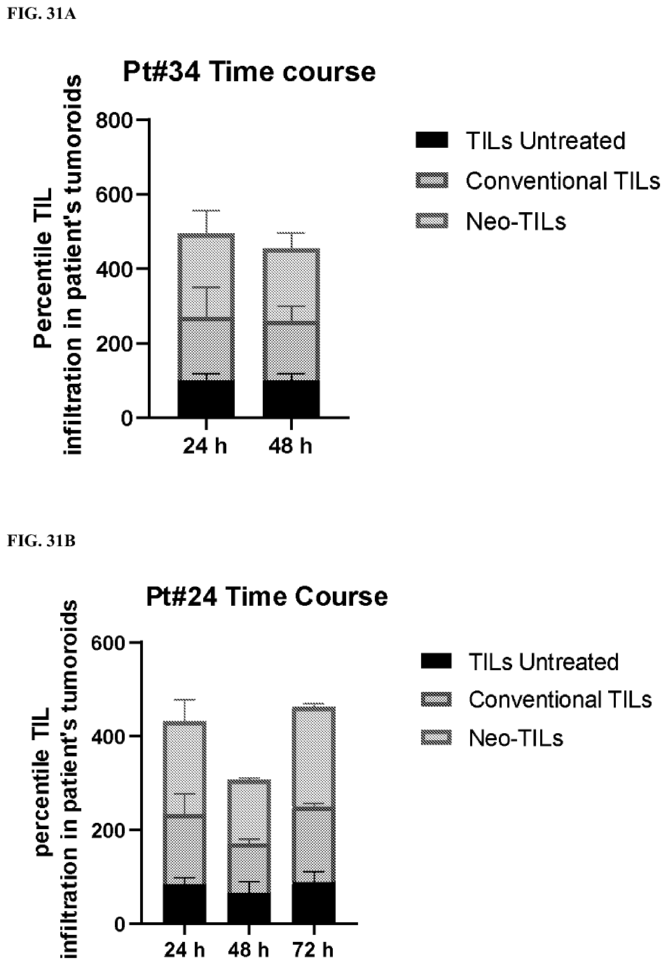

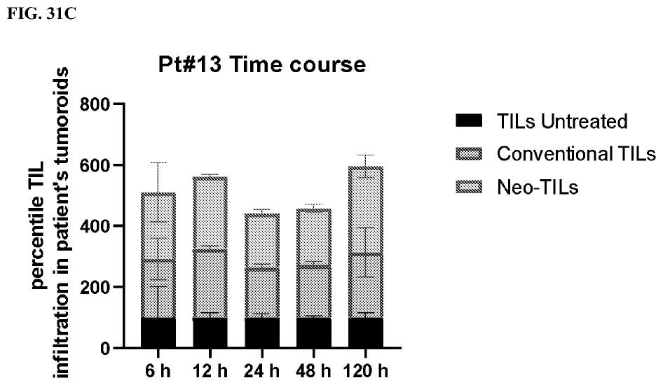

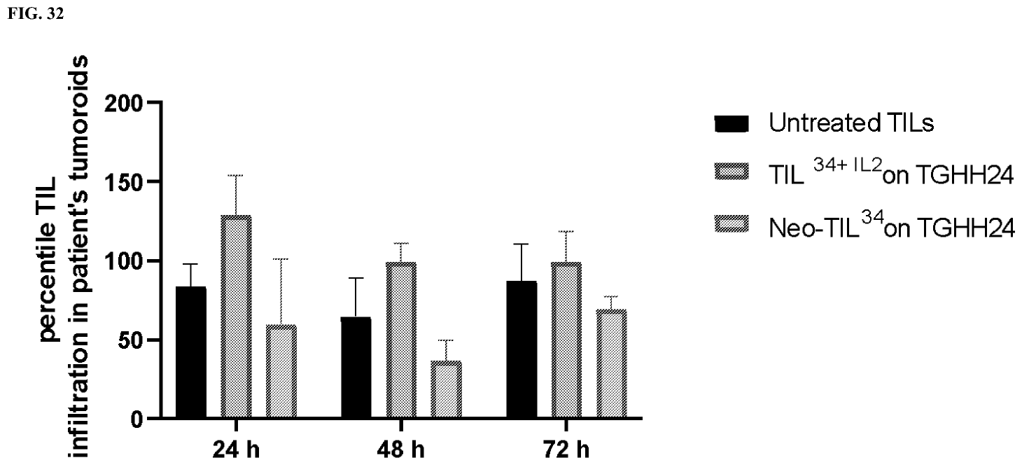

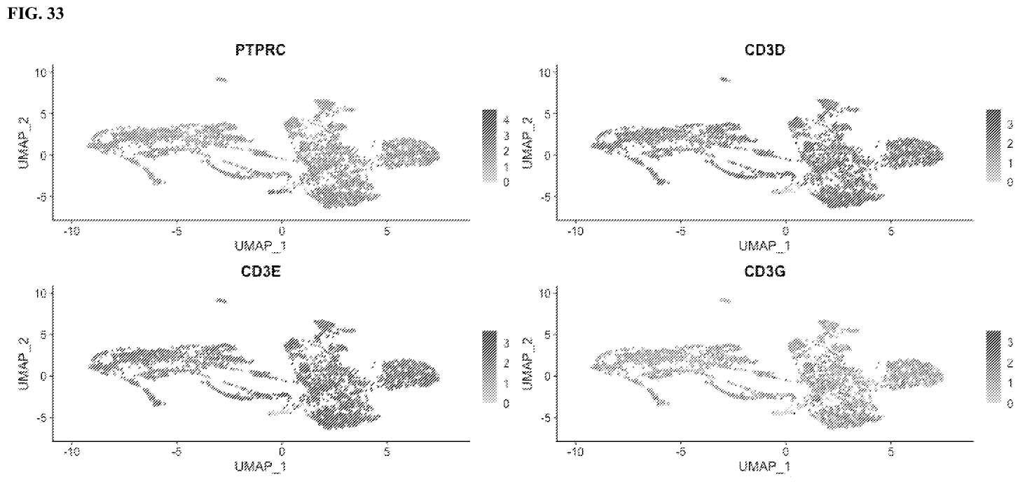

In time course evaluations of immune infiltration, Neo-TIL infiltration was maintained after several days relative to conventional TILs (FIGS. 31A-31C). In addition to maintaining infiltration capabilities, Neo-TILs selectively recognized tumor tissue from the original patient over a three-day time course, whereas conventional TILs recognize tumor tissue from other patients, highlighting the tumor selectivity insured by the Neo-TIL approach (see FIG. 32). In classifying the Neo-TILs, 95% of the cells were CD45 positive, 97% were CD3D positive, 98% were CD3E positive, and 80% were CD3G positive (FIG. 33).

To measure the release of cytokines, TILs were washed prior to use to remove IL-2 that had been used in the rapid expansion protocol. After washing, TILs were cultured overnight with autologous or HLA-mismatched tumor cells at a ratio of 1:1. Supernatant for each co-culture was collected and levels of IFNγ, TNFα, and Granzyme B was measured by the Meso-Scale Discovery (MSD) assay. Gene expression of IFNγ, Perforin, Granzyme B, PD-1, TIM-3, LAG-3, TIGIT were measured by qPCR.

Neo-TILs showed enhanced activation and cytotoxic properties in melanoma as demonstrated by significantly increased levels of IFNγ, TNFα, Granzyme B, and Perforin relative to conventional TILs and untreated TILs (see FIGS. 26C-26E and 27B-G). Further, following rapid expansion Neo-TILs exhibited lower levels of exhaustion markers PD-1, TIM-3, LAG-3, and TIGIT (see FIGS. 27H-27K). Similarly, Neo-TILs exhibited enhanced activation and cytotoxic properties and decreased expression of exhaustion markers in lung cancer (see FIGS. 28C-28G), colon cancer (see FIGS. 29B-29K), and pancreatic cancer (see FIGS. 30B-J).

### Example 11

### Additional Materials and Methods

Dissociation of Primary Human Tumor Tissue into Single-Cell Suspensions for Subsequent TIL Separation

Patients were entered into clinical protocols and signed informed consents that were approved by an Institutional Review Board prior to tumor resection.

Tumors are dissociated and CD45+ TILs are isolated using Milteny Biotech's tumor dissociation kit (#130-095-929) and CD45 (TIL) MicroBeads, human (#130-118-780) respectively. Briefly, fat, fibrous and necrotic areas are removed from the tumor sample. Then the tumor is cut into small pieces of 2-4 mm. The tissue pieces are transferred into the gentleMACS C Tube containing the enzyme mix (the amount of enzymes used depends on the size of tumor as mentioned in manufacturer's protocol). The tissues are the dissociated using gentleMACS dissociator choosing an appropriate gentleMACS™ program. After getting single cell suspension of tumor, cells are incubated with CD45 microbeads and CD45+ cells are isolated by positive selection according to manufacturer's protocol. The CD45+ cells are considered as Tumor infiltrating lymphocytes or TILs and they are frozen using 85% human serum and 15% DMSO till further use.

TILs Cultured with Peptides

After being thawed and washed, 2.5million (if the number of TILs are less, 1 million cells can be used in each condition) TILs are cultured at 37° C. in presence of 2 mL complete RPMI+10% AB serum in each well of a 24 well flat bottom TC treated plate. The complete RPMI media contains RPMI-1640 Medium-Modified, with 20 mM HEPES and L-glutamine, without sodium bicarbonate, liquid, sterile-filtered, suitable for cell culture (Sigma, R7638-500 ml), 10% human AB serum, 10,000 units Penicillin/ml, 10,000 ug Streptomycin/ml), 2 mM L-glutamine, 1 mM Sodium Pyruvate, 1× nonessential amino acids, 5 uM of 2-Mercaptoethanol. Then 0.625 ul of each peptide is added per well containing TILs in 2 ml of complete TIL media (peptide stock concentration is 4 mg/ml in DMSO). Next day, 1 mL of the supernatant is removed from each well and then replaced with fresh IL-2 containing media (100 U/ml) (in IL-2 wells) or TIL media only (in peptide wells) to each well. After every 3 days, cells will be passaged if lot of growth is observed or half of the media will be changed with fresh media. On day 8, cells were lifted by pipetting 10 times and then the wells were washed with 1 ml of 2 mM EDTA-dPBS solution. Finally, the cells are washed and dissolved in TIL media and counted using hemocytometer.

### Rapid Expansion Protocol

T cells can be rapidly expanded using non-specific T-cell receptor Stimulation in the presence of feeder lymphocytes and interleukin-2 (IL-2). The non-specific T-cell receptor stimulus can be induced using around 30 ng/ml of OKT3, a mouse monoclonal anti-CD3 antibody. Briefly, TILs (cultured with peptide) are washed by centrifugation at 1500 rpm for 5 minutes, re-suspended in TIL CM, counted, and viable cells are added to the other components in proportions indicated in Table 3 below:

TABLE 3

| Component | T75 Flask |
|---|---|
| Viable TILs | 5 × [symbol] 10⁵ |
| Feeder PBMCs | 10 [symbol] × 10⁶ |
| OKT3 | 30_ng/ml |
| Recombinant human IL-2 | 6000 IU/ml |
| TIL CM | 15 ml |
| AIM-V CM | [symbol] 15 ml |
| | [symbol] |

On the 5th day after initiating the REP (day 5), half of the media is aspirated from each flask (cells are retained on the bottom of the flask). Media is replaced with TIL CM/AIM-V 50/50 containing 6000 IU/ml IL-2. Then the media is changed in every 2/3 days with TIL CM/AIM-V 50/50 containing 6000 IU/ml IL-2. The culture is continued for 2 weeks. After 2 weeks cells are counted and frozen with 85% human serum (15% DMSO containing) freezing media.

### TIL Efficacy Study

Organoid Generation: Tumor cells are seeded at 5000 cells per well in 96-well ultra-low attachment spheroid microplates (Corning; catalog no. 4591) in 40 μL of appropriate medium containing 1.5% growth factor reduced matrigel (Corning; catalog no. 354230). The plates were then centrifuged at 2000 rpm for 2 mins at RT. Cells were cultured for at least 72 h with 5% CO2 in a humidified incubator to generate spheroids for use in immune infiltration assays.

TIL Infiltration and Imaging: Appropriate number of TILs (normally 2×10⁵ TILs are added to each organoids) are counted after rapid expansion and are centrifuged for 10 min in 1500 rpm at RT. The pellet is then dissolved in 2 ml OptiMem media. In this cell suspension, 2 μM solution of Molecular Probes Vybrant CM-Dil Thermo Fischer; catalog no. C7000) was added and it is incubated for 45 min at 37° C. in the dark and then for an additional 15 min at 4° C. After the incubation, cells were washed with PBS twice and resuspended in the required amount of TIL complete medium. Next, 160 uL of TIL media is added in each well containing tumor organoid. A 5 μm HTS Transwell 96-Well Permeable Support receiver plate (Coming, catalog no. 3387) is then placed on the ultra-low attachment spheroid microplate to allow the TIL infiltration into the organoids. Molecular Probes Vybrant CM-Dil -stained TILs are then seeded into inserts at 2×105 cells/well or at tumoroid:TIL ratio of 1:5. After 24 h, inserts are removed, and organoid microplates are analyzed by 3D Z-stack imaging and morphometric analysis is done with Cytation 5 software to quantify the TIL infiltration.

Cytokine Release Assay: 1×10⁵ TILs are washed prior to use to remove IL-2, then are cultured overnight with autologous or HLA-mismatched tumor cells (if we have any) at a ratio of 1:1. The Supernatant from each co-culture is then assayed for IFN-Y, Granzyme B, IL-4, TNF-a, as per manufacturer's instruction, by MSD plate.

### REFERENCES

¹ Dudley M E, Wunderlich J R, Robbins P F, Yang J C, Hwu P, Schwartzentruber O J, Topalian S L, Sherry R, Restifo N P, Hubicki A M, Robinson M R, Raffeld M, Duray P, Seipp C A, Rogers-Freezer L, Morton K E, Mavroukakis S A, White D E, Rosenberg S A. Cancer regression and autoimmunity in patients after clonal repopulation with antitumor lymphocytes. Science. 2002; 298(5594): 850-4.
² Chandran S S, Somerville R P T, Yang J C, Sherry R M, Klebanoff C A, Goff S L, Wunderlich J R, Danforth O N, Zlott D, Paria B C, Sabesan A C, Srivastava A K, Xi L, Pham T H, Raffeld M, White D E, Toomey M A, Rosenberg S A, Kammula U S. Treatment of metastatic uveal melanoma with adoptive transfer of tumour-infiltrating lymphocytes: a single-centre, two-stage, single-arm, phase 2 study. Lancet Oneal. 2017; 18(6):792-802.
³ Stevanovic S, Draper L M, Langhan M M, Campbell T E, Kwong M L, Wunderlich J R, Dudley M E, Yang J C, Sherry R M, Kammula U S, Restifo N P, Rosenberg S A, Hinrichs C S. Complete regression of metastatic cervical cancer after treatment with human papillomavirus-targeted tumor-infiltrating T cells. J Clin Oneal. 2015; 33(14):1543-50.
⁴ Mehta G U, Malekzadeh P, Shelton T, White D E, Butman J A4, Yang J C, Kammula U S, Goff S L, Rosenberg S A, Sherry R M. Outcomes of Adoptive Cell Transfer With Tumor-infiltrating Lymphocytes for Metastatic Melanoma Patients With and Without Brain Metastases. J lmmunother. 2018 Apr. 18.
⁵ Geukes Foppen M H, Donia M, Svane I M, Haanen J B. Tumor-infiltrating lymphocytes for the treatment of metastatic cancer. Mol Oneal. 2015; 9(10):1918-35.
⁶ Tran E, Ahmadzadeh M, Lu YC, Gras A, Turcotte S, Robbins P F, Gartner J J, Zheng Z, Li Y F, Ray S, Wunderlich J R, Somerville R P, Rosenberg S A. Immunogenicity of somatic mutations in human gastrointestinal cancers. Science. 2015; 350(6266):1387-90.
⁷ Tran E, Turcotte S, Gras A, Robbins P F, Lu Y C, Dudley M E, Wunderlich J R, Somerville R P, Hogan K, Hinrichs C S, Parkhurst M R, Yang J C, Rosenberg S A. Cancer immunotherapy based on mutationspecific CD4+ T cells in a patient with epithelial cancer. Science. 2014; 344(6184):641-5.
⁸ Tran E, Robbins P F, Lu Y C, Prickett T D, Gartner J J, Jia L, Pasetto A, Zheng Z, Ray S, Groh E M, Kriley I R, Rosenberg S A. T -Cell Transfer Therapy Targeting Mutant KRAS in Cancer. N Engl J Med. 2016; 375(23):2255-2262.
⁹ Baitsch L, Baumgaertner P, Devevre E, Raghav S K, Legat A, Barba L, Wieckowski S, Bouzourene H, Deplancke B, Romero P, Rufer N, Speiser D E. Exhaustion of tumor-specific CDS⁺ T cells in metastases from melanoma patients. J Clin Invest. 2011; 121(6):2350-60.

1. A method for expanding tumor infiltrating lymphocytes (TILs) into a therapeutic population of TILs for adoptive cell immunotherapy comprising:
(a) separating a first population of TILs from tumor cells obtained from a tumor resected from a patient;
(b) detecting a plurality of patient-specific tumor mutations in the tumor cells with a genomic analysis of tumor DNA and/or RNA and normal DNA and/or RNA from the patient;
(c) identifying neoantigens resulting from the somatic mutations that demonstrate specific binding with human leukocyte antigen (HLA) proteins or fragments thereof from the first population of TILs;
(d) incubating the neoantigens with the first population of TILs to generate a second population of TILs enriched with lymphocytes recognizing the neoantigens; and
(e) expanding the second population of TILs into a therapeutic population of TILs for adoptive cell immunotherapy.

2. The method of claim 1, wherein detecting a plurality of patient-specific tumor mutations comprises genomic profiling with next generation sequencing of a targeted gene panel.

3. The method of claim 2, wherein the genomic profiling comprises whole genome profiling, whole exome profiling, and/or transcriptome profiling.

4. The method of claim 1, wherein the genomic analysis comprises identifying a plurality of patient-specific tumor mutations in expressed genes by nucleic acid sequencing of tumor and normal samples from the patient and the mutations are present in the genome of cancer cells of the patient but not in normal cells from the subject.

5. The method of claim 1, wherein
the plurality of patient-specific tumor mutations comprises a point mutation, splice-site mutation, frameshift mutation, read-through mutation, gene-fusion mutation, insertion, deletion, or a combination thereof; and
the plurality of patient-specific tumor mutations encodes at least one mutant polypeptide having a tumor-specific neoepitope which binds to an HLA protein or fragment thereof with a greater affinity than a wild-type polypeptide.

6. The method of claim 1, further comprising identifying the MHC class 1 and 2 genotypes of the patient and optionally wherein identifying the MHC class 1 and 2 genotypes of the patient comprises analysis of whole exome sequencing (WES) and/or RNA sequencing from tumor and/or normal tissue.

7. (canceled)

8. The method of claim 1, wherein identifying the neoantigens comprises
(i) providing a library of peptide constructs, wherein
each peptide construct of the library comprises a peptide portion and an identifying nucleic acid portion that identifies the peptide portion, and
the peptide portion of at least one of the peptide constructs is capable of specific binding to the HLA proteins or fragments thereof;
(ii) contacting the HLA proteins or fragments thereof with the library of peptide constructs;
(iii) separating the at least one peptide construct comprising a peptide portion capable of specific binding to the HLA proteins or fragments thereof from peptide constructs comprising a peptide portion not capable of specific binding to the HLA proteins or fragments thereof;
(iv) sequencing all or a portion of the identifying nucleic acid portion of the at least one peptide construct capable of specific binding to the HLA proteins or fragments thereof, and
optionally wherein the library of peptide constructs comprises variant peptides designed from an analysis of the plurality of patient-specific tumor mutations predicting the impact of each mutation on a corresponding protein and excluding silent mutations and mutations in noncoding regions.

9-10. (canceled)

11. The method of claim 1, wherein identifying the neoantigens comprises:
(i) generating a genetically encoded combinatorial library of polypeptides with phage display, ribosomal display, mRNA display, biscistronic DNA display, P2A DNA display, CIS display, yeast display, or bacterial display, wherein the combinatorial library comprises polypeptides linked to corresponding nucleic acid molecules encoding the polypeptides;
(ii) contacting the combinatorial library with the HLA proteins or fragments thereof;
(iii) separating HLA proteins or fragments thereof demonstrating specific binding with the combinatorial library; and
(iv) sequencing all or a portion of the nucleic acid molecules of the combinatorial library bound to the HLA proteins or fragments thereof to identify the neoantigens, and
optionally wherein the combinatorial library of polypeptides comprises variant peptides designed from an analysis of the plurality of patient-specific tumor mutations predicting the impact of each mutation on a corresponding protein and excluding silent mutations and mutations in noncoding regions.

12-13. (canceled)

14. The method of claim 1, wherein specific binding between neoantigens and HLA proteins or fragments thereof from the first population of TILs is determined by:
(i) culturing a cell transformed with at least one nucleic molecule comprising a nucleotide sequence encoding:
an MHC class II component comprising at least a portion of an MHC class II α chain and at least a portion of an MHC class II β chain, such that the MHC class II α chain and MHC class II β chain form a peptide binding groove; and
a spaceholder molecule and a first processable linker, wherein the spaceholder molecule is linked to the MHC class II component by the processable linker and the spaceholder molecule binds within the peptide binding groove thereby hindering the binding of any other peptide within the peptide binding groove; the step of culturing being conducted to produce the MHC class II component;
(ii) recovering the MHC class II component;
(iii) processing the processable linker, thereby releasing the spaceholder molecule from the peptide binding groove;
(iv) incubating the MHC class II component in the presence of a neoantigen, wherein the incubation facilitates the binding of the neoantigen to the peptide binding groove;
(v) recovering the MHC class II component that has bound the neoantigen, and wherein optionally the spaceholder molecule has the consensus sequence AAXAAAAAAAXAA (SEQ ID NO: 78), and/or the spaceholder molecule is selected from the group consisting of PVSKMRMATPLLMQA (SEQ ID NO: 73); AAMAAAAAAAMAA (SEQ ID NO: 74); AAMAAAAAAAAAA (SEQ ID NO: 75); AAFAAAAAAAAAA (SEQ ID NO: 76); and ASMSAASAASMAA (SEQ ID NO: 77).

15-16. (canceled)

17. The method of claim 1, wherein the processable linker is linked to the MHC class II α chain of the MHC class II component and/or wherein recovering the MHC class II component with bound neoantigen comprises affinity chromatography with an antibody recognizing the MHC class II component.

18. (canceled)

19. The method of claim 1, wherein specific binding between neoantigens and HLA proteins or fragments thereof from the first population of TILs is determined by phage display, the HLA proteins or fragments thereof are expressed on the surface of a phage, and the neoantigens are incubated with the phage to assay specific binding and further comprising an in silico analysis to determine specific binding between neoantigens and MHC class I proteins or fragments thereof, wherein the in silico analysis comprises applying a computational algorithm to predict relative binding to MHC I proteins based on the peptide sequences of the neoantigens.

20. (canceled)

21. The method of claim 1, further comprising removing TILs expressing a marker selected from the group consisting of PD1, TIM-3, LAG-3, CTLA-4, and combinations thereof from the first population of TILs and/or the second population of TILs via cell sorting to enrich non-exhausted TILs in the populations, and wherein optionally incubating the neoantigens with the first population of TILs further comprises contacting the first population of TILs with at least one cytokine, the at least one cytokine comprising interleukin-2 (IL-2).

22-23. (canceled)

24. The method of claim 1, wherein expanding the second population of TILs into a therapeutic population of TILs comprises injection of the second population of TILs into a lymph node area of the patient, into the thymus of the patient, and/or systemically into the patient via intravenous administration.

25. The method of claim 1, wherein expanding the second population of TILs into a therapeutic population of TILs comprises supplementing the cell culture medium of the second population of TILs with IL-2, optionally OKT-3, and feeder cells.

26. The method of claim 1, wherein expanding the second population of TILs into a therapeutic population of TILs comprises:
(i) performing a first expansion by culturing the second population of TILs in a cell culture medium comprising IL-2, and optionally OKT-3, to produce a third population of TILs, wherein the first expansion is performed in a closed container providing a first gas-permeable surface area, wherein the first expansion is performed for about 3-14 days to obtain the third population of TILs, wherein the third population of TILs is at least 50-fold greater in number than the second population of TILs, and wherein the transition occurs without opening the system; and, optionally,
(ii) performing a second expansion by supplementing the cell culture medium of the third population of TILs with additional IL-2, optionally OKT-3, and feeder cells, to produce a fourth population of TILs, wherein the second expansion is performed for about 7-14 days to obtain the fourth population of TILs, wherein the fourth population of TILs is a therapeutic population of TILs, wherein the third expansion is performed in a closed container providing a second gas-permeable surface area, and wherein the transition occurs without opening the system, and
wherein optionally the feeder cells are antigen presenting cells (APCs) or irradiated peripheral blood mononuclear cells (PBMCs).

27. (canceled)

28. The method of claim 1, further comprising performing an immune infiltration assay with organoids derived from tumor cells from the patient to confirm enhanced infiltration of the organoids with the second population of TILs compared to the first population of TILs.

29. A cryopreserved composition comprising the therapeutic population of TILs of claim 1, a cryoprotectant medium comprising DMSO, and an electrolyte solution and further comprising one or more stabilizers and one or more lymphocyte growth factors, and optionally the one or more stabilizers comprise Human Serum Albumin (HSA) and the one or more lymphocyte growth factors comprise IL-2.

30-31. (canceled)

32. A method of treating a subject with cancer, the method comprising administering an effective amount of the therapeutic population of TILs of claim 1 to a patient in need thereof.

33. The method of claim 32, wherein prior to administering an effective amount of the therapeutic population of TILs, a non-myeloablative lymphodepletion regimen is administered to the patient, the non-myeloablative lymphodepletion regimen comprises the steps of administration of cyclophosphamide at a dose of 60 mg/m²/day for two days followed by administration of fludarabine at a dose of 25 mg/m²/day for five days.

34. (canceled)

35. The method of claim 32, further comprising the step of treating the patient with a high-dose IL-2 regimen starting on the day after administration of the therapeutic population of TILs, wherein the high-dose IL-2 regimen comprises 600,000 or 720,000 IU/kg administered as a 15-minute bolus intravenous infusion every eight hours until tolerance; and/or further comprising contacting the therapeutic population of TILs with or administering to the patient an immune checkpoint inhibitor, wherein the checkpoint inhibitor comprises a CD27, CD28, CD40, CD 122, CD137, OX40, GITR, ICOS, A2AR, B7-H3, B7-H4, BTLA, CTLA-4, IDO, MR, LAG3, PD-1, PD-L1, TIM-2, 4-1BB, or VISTA inhibitor or a combination thereof, and/or wherein the checkpoint inhibitor comprises ipiliumab (anti-CTLA-4), penbrolizumab (anti-PD-L1), nivolumab (anti-PD-L1), atezolizumab (anti-PD-L1), duralumab (anti-PD-L1), or a combination thereof.

36-42. (canceled)
